(19) United States
(12) Patent Application Publication
(10) Pub. No.: US 2011/0245284 A1
(43) Pub. Date: Oct. 6, 2011

(54) Alkoxy- and Alkylthio-Substituted Anilinopyrimidines

(75) Inventors: Jörg Nico Greul, Leverkusen (DE); Hendrik Helmke, Liederbach (DE); Stefan Hillebrand, Neuss (DE); Amos Mattes, Langenfeld (DE); Pierre Wasnaire, Dusseldorf (DE); Carl Friedrich Nising, Langenfeld (DE); Ulrike Wachendorff-Neumann, Neuwied (DE); Peter Dahmen, Neuss (DE); Arnd Voerste, Koln (DE); Ruth Meissner, Leverkusen (DE); Chirstoph Andreas Braun, Dusseldorf (DE); Martin Kaubmann, Koln (DE); Hiroyuki Hadano, Tochigi (JP); Ulrich Heinemann, Leichlingen (DE)

(73) Assignee: Bayer CropScience AG, Monheim (DE)

(21) Appl. No.: 13/061,896

(22) PCT Filed: Aug. 21, 2009
(86) PCT No.: PCT/EP2009/006057

§ 371 Date: Jun. 23, 2011

(30) Foreign Application Priority Data
Sep. 3, 2008 (EP) 08163607.8

(51) Int. Cl. A01N 43/54; C07D 239/48; C07D 239/42; C07C 205/11; C07C 321/20; A01P 3/00

(52) U.S. Cl. 514/275; 544/323; 544/327; 544/326; 544/330; 568/936; 564/440

(57) ABSTRACT

Alkoxy- and alkylthio-substituted anilinopyrimidines of the formula (I)

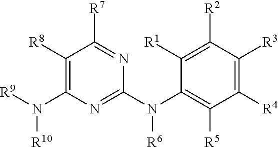

in which R¹ to R¹⁴ and E1, E2, E3, X and Y have the meanings given in the description, and agrochemically active salts thereof, their use, and methods and compositions for controlling phytopathogenic harmful fungi in and/or on plants or in and/or on seeds of plants, processes for preparing such compositions and treated seeds and also their use for controlling phytopathogenic hal inful fungi in agriculture, horticulture and forestry, in the protection of materials and in the domestic and hygiene field. The present invention furthermore relates to a process for the preparation of alkoxy- and alkylthio-substituted anilinopyrimidines of the formula (I).

The invention relates to alkoxy- and alkylthio-substituted anilinopyrimidines and their agrochemically active salts, to their use and to methods and compositions for controlling phytopathogenic harmful fungi in and/or on plants or in and/or on seeds of plants, to processes for preparing such compositions and treated seeds and also to their use for controlling phytopathogenic harmful fungi in agriculture, horticulture and forestry, in the protection of materials and in the domestic and hygiene field. The present invention furthermore relates to a process for preparing alkoxy- and alkylthio-substituted aniliminopyrimidines.

It is already known that certain alkynyl-substituted diaminopyrimidines can be used as fungicidal crop protection agents (see DE 4029650 A1). However, in particular at low application rates, the fungicidal activity of these compounds is not always sufficient.

Since the ecological and economic demands made on modern crop protection agents are increasing constantly, for example with regard to spectrum of action, toxicity, selectivity, application rate, formation of residues and favourable manufacture, and since furthermore for example resistance problems may occur, there is a constant need to develop novel crop protection agents, in particular fungicides, which have advantages over the known ones, at least in some areas.

Surprisingly, it has now been found that the present alkoxy- and alkylthio-substituted diaminopyrimidines solve at least some aspects of the objects mentioned and are suitable for use as crop protection agents, in particular as fungicides.

Some of these alkoxy- and alkylthio-substituted anilinopyrimidines are already known as pharmaceutically active compounds (see, for example, WO 06/021544, WO 07/072158, WO 07/003596, WO 05/016893, WO 05/013996, WO 04/056807, WO 04/014382, WO 03/030909), but not their surprising fungicidal activity.

The invention provides compounds of the formula (I)

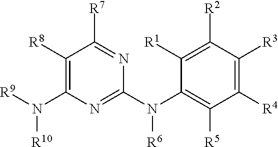

in which one or more of the symbols have one of the following meanings:
R¹ to R⁵ independently of one another are hydrogen, C₁-C₄-alkyl, C₁-C₄-alkoxy, C₁-C₄-haloalkyl, C₁-C₄-alkoxy(C₁-C₄)alkyl, C₁-C₄-alkoxy(C₁-C₄)alkoxy or halogen,
   where precisely one of the radicals R² or R³ resents a group of the formula E1, E2 or E3,

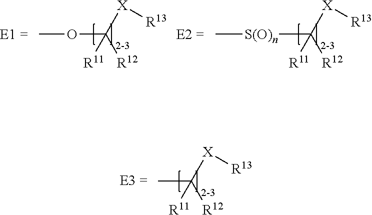

in which one or more of the symbols have one of the following meanings:
X is oxygen, NR¹⁴, sulphur, SO or SO₂,
Y is a direct bond, oxygen, NR¹⁴ sulphur, SO or SO₂,
n is 0, 1 or 2,
R⁶ is hydrogen, C₁-C₂-alkyl, C₁-C₄-alkoxy(C₁-C₄)alkyl, C₁-C₄-trialkylsilyl, C₁-C₄-trialkylsilylethyl, C₁-C₄-dialkylmonophenylsilyl, formyl, (C₁-C₄-alkyl)carbonyl, (C₁-C₄-alkoxy-C₁-C₄-alkyl)carbonyl, (C₃-C₆-alkenyloxy)carbonyl, (C₃-C₆-cycloalkyl)carbonyl, (halo-C₁-C₄-alkoxy-C₁-C₄-alkyl)carbonyl, (C₁-C₄-haloalkyl)carbonyl, (C₁-C₄-alkoxy)carbonyl, (C₁-C₄-haloalkoxy)carbonyl, benzyloxycarbonyl, unsubstituted or substituted benzyl, unsubstituted or substituted C₂-C₆-alkenyl, unsubstituted or substituted C₂-C₆-alkynyl, C₁-C₂-alkylsulphinyl or C₁-C₂-alkylsulphonyl,
   where the substituents independently of one another are selected from among hydrogen, fluorine, chlorine or bromine, C₁-C₄-alkyl, C₁-C₄-alkoxy, hydroxyl, C₁-C₄-haloalkyl, or cyano,
R⁷ is hydrogen, C₁-C₃-alkyl, cyano or C₁-C₃-haloalkyl,
R⁸ is methyl, fluorine, chlorine, bromine, SMe, SOMe, SO₂Me, iodine, CCl₃, CH₂F, CHF₂ or CF₃,
R⁹ is hydrogen, unbranched or branched C₁-C₃-alkyl, 2-methoxyethan-1-yl, prop-2-en-1-yl, C₁-C₄-alkoxy(C₁-C₄)alkyl, unbranched or branched (C₁-C₄-alkyl)carbonyl, (C₁-C₄-haloalkyl)carbonyl, unsubstituted or substituted benzyl, C₁-C₆-trialkylsilyl, C₁-C₄-trialkylsilylethyl, C₁-C₄-dialkylmonophenylsilyl, (C₁-C₄-alkoxy)carbonyl, C₁-C₆-alkylsulphinyl, C₁-C₆-alkylsulphonyl, C₁-C₆-haloalkylsulphinyl or C₁-C₆-haloalkylsulphonyl,
   where the substituents independently of one another are selected from among hydrogen, halogen, nitro, C₁-C₄-alkyl, C₁-C₄-alkoxy, hydroxyl, C₁-C₄-haloalkyl or cyano
R¹⁰ is unbranched or branched, unsubstituted or substituted C₁-C₇-alkyl, unbranched or branched, unsubstituted or substituted C₂-C₇-haloalkyl, unsubstituted or substituted C₃-C₇-cycloalkyl, unbranched or branched, unsubstituted or substituted C₃-C₇-cycloalkyl(C₁-C₃)alkyl, unbranched or branched, unsubstituted or substituted C₃-C₇-alkenyl, unbranched or branched, unsubstituted or substituted C₃-C₇-alkynyl, unbranched or branched, unsubstituted or substituted C₁-C₄-alkoxy(C₁-C₄)alkyl, unbranched or branched, unsubstituted or substituted C₁-C₄-haloalkoxy(C₁-C₄)alkyl, 2-methyl-1-(methylsulphanyl)propan-2-yl or oxetan-3-yl,
or
R⁹ and R¹⁰ together with the nitrogen atom to which they are bonded form an unsubstituted or substituted 3-7-membered saturated cycle which may contain up to one further heteroatom selected from among oxygen, sulphur or nitrogen,
   where the substituents in R¹⁰ independently of one another are selected from among methyl, ethyl, isopropyl, cyclopropyl, fluorine atoms, chlorine atoms and/or bromine atoms, methoxy, ethoxy, methylmercapto, ethylmercapto, cyano, hydroxyl, CF₃,
R¹¹ and R¹² independently of one another are hydrogen, halogen, C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
or
R¹¹ and R¹² together form a methylene group ═CH₂,

R¹³ is hydrogen, C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,

where the substituents independently of one another are selected from among halogen, C₁-C₄-alkyl or C₁-C₄-haloalkyl,
R¹⁴ is hydrogen, C₁-C₆-alkyl, C₁-C₄-alkoxy(C₁-C₄)alkyl or unsubstituted or substituted benzyl,
   where the substituents independently of one another are selected from among halogen, C₁-C₄-alkyl or C₁-C₄-haloalkyl,
   and agrochemically active salts thereof.

A further subject matter is the use of the compounds of the formula (I) as fungicides.

Diaminopyridines of the formula (I) according to the invention and their agrochemically active salts are highly suitable for controlling phytopathogenic harmful fungi. The compounds according to the invention mentioned above have in particular potent fungicidal activity and can be used in crop protection, in the domestic and hygiene fields and in the protection of materials.

The compounds of the formula (I) can be present both in pure form and as mixtures of various possible isomeric forms, in particular of stereoisomers, such as E and Z, threo- and erythro-, and also optical isomers, such as R and S isomers or atropisomers, and, if appropriate, also of tautomers. What is claimed are both the E and the Z isomers, and the threo- and erythro-, and also the optical isomers, any mixtures of these isomers, and also the possible tautomeric forms.

Preference is given to compounds of the formula (I) in which one or more of the symbols have one of the following meanings:
R¹ to R⁵ independently of one another are hydrogen, C₁-C₃-alkyl, C₁-C₃-alkoxy, C₁-C₂-haloalkyl, C₁-C₄-alkoxy(C₁-C₄)alkyl, C₁-C₄-alkoxy(C₁-C₄)alkoxy or halogen,
   where precisely one of the radicals R² or R³ represents a group of the formula E1, E2 or E3,

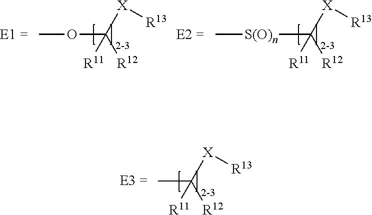

in which one or more of the symbols have one of the following meanings:
X is oxygen, NR¹⁴, sulphur, SO or SO₂,
Y is a direct bond, oxygen, NR¹⁴, sulphur, SO or SO₂,
n is 0, 1 or 2,
R⁶ is hydrogen, C₁-C₂-alkyl, C₁-C₄-alkoxy(C₁-C₄)alkyl, C₁-C₄-trialkylsilyl, formyl, (C₁-C₄-alkyl)carbonyl, (C₁-C₄-alkoxy-C₁-C₄-alkyl)carbonyl, (C₃-C₆-cycloalkyl)carbonyl, (C₁-C₄-haloalkyl)carbonyl, (C₁-C₄-alkoxy)carbonyl, benzyloxycarbonyl, unsubstituted or substituted benzyl, unsubstituted or substituted C₂-C₆-alkenyl, unsubstituted or substituted C₂-C₆-alkynyl or C₁-C₂-alkylsulphonyl,
   where the substituents independently of one another are selected from among fluorine, chlorine or bromine, C₁-C₂-alkyl, C₁-C₂-alkoxy, hydroxyl, C₁-C₂-haloalkyl, or cyano,
R⁷ is hydrogen, C₁-C₃-alkyl, cyano or C₁-C₃-haloalkyl,
R⁸ is methyl, fluorine, chlorine, bromine, SMe, SOMe, SO₂Me, iodine, CCl₃, CH₂F, CHF₂ or CF₃,
R⁹ is hydrogen, methyl, ethyl, propyl, propan-2-yl, butyl, pentyl, hexyl, 2-methoxyethan-1-yl, 2,2,2-trifluoroethyl, prop-2-en-1-yl, CH₂OCH₃, COMe, COOMe, COOEt, COOtertBu, COCF₃, benzyl or SO₂CH₃,
R¹⁰ is unbranched or branched, unsubstituted or substituted C₁-C₆-alkyl, unbranched or branched, unsubstituted or substituted C₃-C₆-cycloalkyl(C₁-C₂)alkyl, unsubstituted or substituted C₃-C₆-cycloalkyl, unbranched or branched, unsubstituted or substituted C₃-C₄-alkenyl, unbranched or branched, unsubstituted or substituted C₃-C₄alkynyl, unbranched or branched, unsubstituted or substituted C₂-C₄-haloalkyl, unbranched or branched, unsubstituted or substituted C₁-C₂-alkoxy(C₁-C₄)alkyl, unbranched or branched, unsubstituted or substituted C₁-C₂-alkylmercapto(C₁-C₄)alkyl or oxetan-3-yl,
   where the substituents in R¹⁰ independently of one another are selected from among methyl, ethyl, isopropyl, cyclopropyl, fluorine atoms, chlorine atoms and/or bromine atoms, methoxy, ethoxy, methylmercapto, ethylmercapto, cyano, hydroxyl, CF₃
or
R⁹ and R¹⁰ together with the nitrogen atom to which they are bonded form an azetidinyl, pyrrolidinyl, piperidinyl, morpholinyl, azepanyl, 4-methylpiperazin-1-yl, 2-methylpiperidin-1-yl, -methylpyrrolidin-1-yl, 2-methylazetidin-1-yl or thiomorpholinyl ring,
R¹¹ and R¹² independently of one another are hydrogen, halogen, C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
or
R¹¹ and R¹² together form a methylene group ═CH₂,
R¹³ is hydrogen, C₁-₆-alkyl, unsubstituted or substituted C₃-C₆-cycloalkyl, unsubstituted or substituted C₃-₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
R¹⁴ is hydrogen, C₁-C₆-alkyl, C₁-C₄-alkoxy(C₁-C₄)alkyl or unsubstituted or substituted benzyl,
   where the substituents independently of one another are selected from among halogen, C₁-C₄-alkyl or C₁-C₄-haloalkyl,
   and the agrochemically active salts thereof.

Especially preferred compounds of the formula (I) are those in which one or more of the symbols have one of the following meanings:
R¹ to R⁵ independently of one another are hydrogen, methyl, ethyl, fluorine, chlorine, bromine, iodine, trifluoromethyl, difluoromethyl, OCH₃, OCH₂CH₃, O(CH₂)₂OCH₃, CH₂OCH₃ or CH₂OCH₂CH₃,
   where precisely one of the radicals R² or R³ represents a group of the formula E1, E2 or E3,

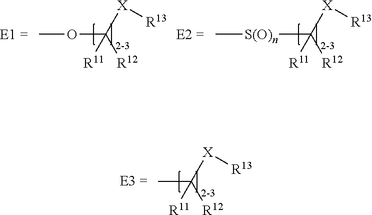

in which one or more of the symbols have one of the following meanings:
X is oxygen, NR¹⁴, sulphur, SO or SO₂,
Y is a direct bond, oxygen, NR¹⁴, sulphur, SO or SO₂,
n is 0, 1 or 2,
R⁶ is hydrogen, Me, benzyl, SO₂CH₃, COMe, COCF₃, COOMe or CHO,
R⁷ is hydrogen, methyl, cyano, CHF₂, or CF₃,
R⁸ is methyl, fluorine, chlorine, bromine, SMe, SOMe, SO₂Me, iodine, CCl₃, CHF₂ or CF₃,
R⁹ is hydrogen, methyl, ethyl, propyl, propan-2-yl, butyl, pentyl, hexyl, 2-methoxyethan-1-yl, 2,2,2-trifluoroethyl, prop-2-en-1-yl, CH₂OCH₃, COMe, COOMe, COOEt, COOtertBu, COCF₃, benzyl or SO₂CH₃,
R¹⁰ is methyl, ethyl, propyl, isopropyl, butyl, tent-butyl, 2-methylprop-1-yl, butan-2-yl, pentyl, 2,2-dimethylprop-1-yl, 2-methylbut-1-yl, 3-methylbut-1-yl, 3-methylbutan-2-yl, pentan-2-yl, pentan-3-yl, hexyl, 2,2-dimethylbutan-2-yl, prop-2-en-1-yl, 2-methylprop-2-en-1-yl, prop-2-yn-1-yl, 2-fluoro ethan-1-yl, 1-fluoropropan-2-yl, 3-fluoropropan-1-yl, 2,2-difluoro-ethyl, 2,2,2-trifluoroethyl, 2,2-difluoropropan-1-yl, 1,1,1-trifluoropropan-2-yl, 3,3,3-trifluoropropan-1-yl, 2,2,3,3,3-pentafluoropropyl, 1,1,1-trifluorobutan-2-yl, 1,1,1-trifluorobutan-3-yl, 1,1,1-trifluoro-2-methylpropan-2-yl, 1-fluoro-2-methylpropan-2-yl, 1,1,1-trifluoro-3-methylbutan-2-yl, 2-chloroethan-1-yl, cyanomethyl, 2-methoxyethan-1-yl, 3-methoxypropan-1-yl, 2-methylmercaptoethan-1-yl, 1-methylmercaptopropan-2-yl, cyclopropyl, cyclobutyl, cyclopentyl, cyclohexyl, oxetan-3-yl, cyclopropylmethyl, 1-cyclopropyleth-1-yl, 2-methylcyclopropyl, 2,2-dimethylcyclopropyl, 2-methylcyclo-but-1-yl, 3-methylcyclobut-1-yl, 2-methyl-3-oxobutan-2-yl or 3-(2-oxoazepan-1-yl)propyl,
or
R⁹ and R¹⁰ together with the nitrogen atom to which they are bonded form an aziridinyl, azetidinyl, pyrrolidinyl, piperidinyl, azepanyl, piperazin-1-yl, 4-methylpiperazin-1-yl, morpholinyl or thiomorpholinyl ring,
R¹¹ and R¹² independently of one another are hydrogen, methyl, ethyl, propyl, isopropyl, butyl, pentyl, hexyl, cyclopropyl, cyclobutyl, cyclopentyl, cyclohexyl, chlorine, trifluoromethyl, (CH₂)₂OCH₃, phenyl or benzyl,
or
R¹¹ and R¹² together form a methylene group ═CH₂,
R¹³ is hydrogen, methyl, ethyl, propyl, isopropyl, butyl, pentyl, hexyl, 2,2,2-trifluoroethyl, cyclopropyl, cyclobutyl, cyclopentyl, cyclohexyl, (CH₂)₂OCH₃, phenyl or benzyl,
R¹⁴ is hydrogen, methyl, ethyl, propyl, isopropyl, butyl, pentyl, (CH₂)₂OCH₃ or benzyl,
   and the agrochemically active salts thereof.

Very especially preferred compounds of the formula (I) are those in which one or more of the symbols have one of the following meanings:
R¹ to R⁵ independently of one another are hydrogen, methyl, ethyl, fluorine, chlorine, bromine, iodine, trifluoromethyl, difluoromethyl, OCH₃, OCH₂CH₃, O(CH₂)₂OCH₃, CH₂OCH₃ or CH₂OCH₂CH₃,
   where precisely one of the radicals R² or R³ represents a group of the formula E1, E2 or E3,

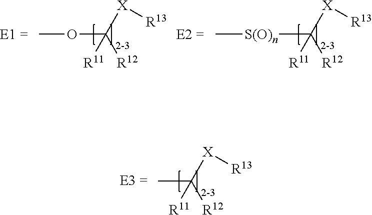

in which one or more of the symbols have one of the following meanings:
X is oxygen, sulphur, SO or SO₂,
Y is a direct bond, oxygen, sulphur, SO or SO₂,
n is 0, 1 or 2,
R⁶ is hydrogen, Me, COMe or CHO,
R⁷ is hydrogen or methyl,
R⁸ is methyl, fluorine, chlorine, bromine, SMe, SOMe, SO₂Me, CHF₂ or CF₃,
R⁹ is hydrogen, methyl, ethyl, propyl, propan-2-yl, butyl, pentyl, 2-methoxyethan-1-yl, 2,2,2-trifluoroethyl, prop-2-en-1-yl, CH₂OCH₃, COMe, COOMe, COOEt, COOtertBu, COCF₃ or benzyl,
R¹⁰ is methyl, ethyl, propyl, isopropyl, butyl, tert-butyl, 2-methylprop-1-yl, butan-2-yl, pentyl, 2,2-dimethylprop-1-yl, 2-methylbut-1-yl, 3-methylbut-1-yl, pentan-2-yl, pentan-3-yl, hexyl, prop-2-en-1-yl, 2-methylprop-2-en-1-yl, prop-2-yn-1-yl, 2-fluoroethan-1-yl, 1-fluoropropan-2-yl, 2,2-difluoroethyl, 2,2,2-trifluoroethyl, 1,1,1-trifluoropropan-2-yl, 3,3,3-trifluoropropan-1-yl, 2,2,3,3,3-pentafluoropropyl, 1,1,1-trifluorobutan-2-yl, 1,1,1-trifluorobutan-3-yl, 2-chloroethan-1-yl, cyanomethyl, 2-methoxyethan-1-yl, 3-methoxypropan-1-yl, 2-methylmercaptoethan-1-yl, 1-methylmercaptopropan-2-yl, cyclopropyl, cyclobutyl, cyclopentyl, cyclohexyl, oxetan-3-yl, cyclopropylmethyl, 1-cyclopropyleth-1-yl, 2-methylcyclopropyl, 2,2-dimethylcyclopropyl, 2-methylcyclobut-1-yl, 3-methylcyclobut-1-yl, 2-methyl-3-oxobutan-2-yl or 3-(2-oxoazepan-1-yl)propyl,
or
R⁹ and R¹⁰ together with the nitrogen atom to which they are bonded form an aziridinyl, azetidinyl, pyrrolidinyl, piperidinyl, azepanyl, piperazin-1-yl, 4-methylpiperazin-1-yl, morpholinyl or thiomorpholinyl ring,

R¹¹ and R¹² independently of one another are hydrogen, methyl, ethyl, propyl, isopropyl, butyl, pentyl, hexyl, chlorine, cyclopropyl, trifluoromethyl, phenyl or benzyl,
or
R¹¹ and R¹² together form a methylene group ═CH₂,
R¹³ is hydrogen, methyl, ethyl, propyl, isopropyl, butyl, pentyl, hexyl, 2,2,2-trifluoroethyl, (CH₂)₂OCH₃, phenyl or benzyl
   and the agrochemically active salts thereof.

Especially preferred compounds of the formula (I) are those in which one or more of the symbols have one of the following meanings
R¹ and R⁵ independently of one another are hydrogen or F,
R² is hydrogen, fluorine, chlorine, trifluoromethyl, O(CH₂)₂OCH₃, O(CH₂)₂OCH₂CH₃, O(CH₂)₂OCH₂CH₂CH₃, O(CH₂)₃OCH₃, O(CH₂)₃OCH₂CH₃, O(CH₂)₂O(CH₂)₂OCH₃, (1-methoxypropan-2-yl)oxy, CH₂OCH₃, CH₂OCH₂CH₃, CH₂OCH₂CH₂CH₃, 1-methoxy-ethyl, 1-ethoxyethyl, 1-propoxyethyl, 2,2,2-trifluoro-1-methoxyethyl, 2,2,2-trifluoro-1-ethoxyethyl, 2-methoxypropan-2-yl, 2-ethoxypropan-2-yl, phenoxymethyl, (CH₂)₂OCH₃, (CH₂)₂OCH₂CH₃, 2-methoxy-2-methylpropyl, 2-ethoxy-2-methylpropyl, SEt, SOEt, SO₂Et, SPr, SOPr, SO₂Pr, SisoPr, SOisoPr, SO₂isoPr, SBu, SOBu, SO₂Bu, SisoBu, SOisoBu, SO₂isoBu, SsecBu, SOsecBu, SO₂secBu, (2-methylprop-2-en-1-yl)sulphanyl, (2-chloroethyl)sulphonyl, (methylsulphanyl)methyl, (methylsulphinyl)methyl, (methylsulphonyl)methyl, (ethylsulphanyl)methyl, (ethylsulphinyl)methyl, (ethylsulphonyl)-methyl, (propylsulphanyl)methyl, (propylsulphinyl)methyl, (propylsulphonyl)methyl, 1-(methylsulphanyl)ethyl, 1-(methylsulphinyl)ethyl, 1-(methylsulphonyl)ethyl, 1-(ethylsulphanyl)ethyl, 1-(ethylsulphinyl)ethyl, 1-(ethylsulphonyl)ethyl, 1-(propylsulphanyl)ethyl, 1-(propyl sulphinyl)ethyl, 1-(propylsulphonyl)ethyl, 1-(isopropylsulphanyl)ethyl, 1-(isopropylsulphinyl)ethyl, 1-(isopropylsulphonyl)ethyl, 1-(sec-butylsulphanyl)ethyl, 1-(sec-butylsulphinyl)ethyl, 1-(sec-butylsulphonyl)ethyl, 1-(pentan-2-ylsulphanyl)ethyl, 1-(pentan-2-yl sulphinyl)ethyl, 1-(pentan-2-ylsulphonyl)ethyl, 1-(methylsulphanyl)propyl, 1-(methylsulphinyl)propyl, 1-(methylsulphonyl)propyl, 1-(ethylsulphanyl)propyl, 1-(ethylsulphinyl)propyl, 1-(ethylsulphonyl)propyl, 2-(methylsulphanyl)ethyl, 2-(methylsulphinyl)ethyl, 2-(methylsulphonyl)ethyl, 2-(ethylsulphanyl)ethyl, 2-(ethylsulphinyl)ethyl or 2-(ethylsulphonyl)ethyl,
R³ is hydrogen, methyl, fluorine, chlorine, bromine, trifluoromethyl, O(CH₂)₂OCH₃, O(CH₂)₂OCH₂CH₃, O(CH₂)₃OCH₃, O(CH₂)₃OCH₂CH₃, O(CH₂)₂O(CH₂)₂OCH₃, (1-methoxypropan-2-yl)oxy, CH₂OCH₃, CH₂OCH₂CH₃, CH₂OCH₂CH₂CH₃, 1-methoxyethyl, 1-ethoxyethyl, 1-propoxyethyl, 2-methoxypropan-2-yl, 2-ethoxypropan-2-yl, (CH₂)₂OCH₃, (CH₂)₂OCH₂CH₃, 2-methoxy-2-methylpropyl, 2-ethoxy-2-methylpropyl, SEt, SOEt, SO₂Et, SPr, SOPr, SO₂Pr, SisoPr, SOisoPr, SO₂isoPr, SBu, SOBu, SO₂Bu, SisoBu, SOisoBu, SO₂isoBu, SsecBu, SOsecBu, SO₂secBu, (methylsulphanyl)methyl, (methylsulphinyl)methyl, (methylsulphonyl)methyl, (ethylsulphanyl)methyl, (ethylsulphinyl)methyl, (ethylsulphonyl)methyl, 1-(methylsulphanyl)ethyl, 1-(methylsulphinyl)ethyl, 1-(methylsulphonyl)ethyl, 1-(ethylsulphanyl)ethyl, 1-(ethylsulphinyl)ethyl, 1-(ethylsulphonyl)ethyl, 1-(propylsulphanyl)ethyl, 1-(propylsulphinyl)ethyl, 1-(propylsulphonyl)ethyl, 1-(methylsulphanyl)propyl, 1-(methylsulphinyl)propyl, 1-(methylsulphonyl)propyl, 1-(ethylsulphanyl)propyl, 1-(ethylsulphinyl)propyl, 1-(ethylsulphonyl)propyl, 2-(methylsulphanyl)ethyl, 2-(methylsulphinyl)ethyl or 2-(methylsulphonyl)ethyl,
   where R² and R³ are not simultaneously hydrogen,
   with the proviso that, if R² is other than hydrogen, fluorine, chlorine or trifluoromethyl,
R³ may only have one of the following meanings:
   hydrogen, methyl, fluorine, chlorine, bromine or trifluoromethyl,
R⁴ is hydrogen, methyl, fluorine, chlorine, trifluoromethyl, O(CH₂)₂OCH₃, CH₂OCH₃ or CH₂OCH₂CH₃,
R⁶ is hydrogen or CHO,
R⁷ is hydrogen,
R⁸ is fluorine, chlorine, bromine, SMe, SOMe, SO₂Me or CF₃,
R⁹ is hydrogen, methyl, ethyl, propyl, propan-2-yl, 2-methoxyethan-1-yl or prop-2-en-1-yl,
R¹⁰ is methyl, ethyl, propyl, isopropyl, butyl, tert-butyl, 2-methylprop-1-yl, pentyl, 2,2-dimethylprop-1-yl, 2-methylbut-1-yl, 3-methylbut-1-yl, pentan-2-yl, pentan-3-yl, prop-2-en-1-yl, prop-2-yn-1-yl, 2-fluoroethan-1-yl, 2,2-difluoroethyl, 2,2,2-trifluoroethyl, 2-chloroethan-1-yl, cyanomethyl, cyclopropyl, cyclobutyl, cyclopentyl, cyclopropylmethyl, 1-cyclopropyleth-1-yl, 2-methylcyclopropyl, 2-methyl-3-oxobutan-2-yl or 3-(2-oxoazepan-1-yl)propyl,
or
R⁹ and R¹⁰ together with the nitrogen atom to which they are bonded form a pyrrolidinyl, piperidinyl, azepanyl, 4-methylpiperazin-1-yl, morpholinyl or thiomorpholinyl ring,
   and the agrochemically active salts thereof.

Furthermore, especially preferred compounds of the formula (I) are those in which one or more of the symbols have one of the following meanings:
R¹ and R⁵ independently of one another are hydrogen,
R² is hydrogen, fluorine, chlorine, trifluoromethyl, O(CH₂)₂OCH₃, O(CH₂)₂OCH₂CH₃, CH₂OCH₃, 2,2,2-trifluoro-1-methoxyethyl, 2-methoxypropan-2-yl, phenoxymethyl, 2-methoxy-2-methylpropyl, SOEt, SO₂Et, SPr, SOPr, SO₂Pr, SsecBu, (2-methylprop-2-en-1-yl)sulphanyl, (2-chloroethyl)sulphonyl, (ethylsulphanyl)methyl, 1-(methylsulphanyl)ethyl, 1-(ethylsulphanyl)ethyl, 1-(ethylsulphinyl)ethyl, 1-(ethylsulphonyl)ethyl or 1-(methylsulphanyl)propyl,
R³ is hydrogen, methyl, chlorine, O(CH₂)₂OCH₃, O(CH₂)₂OCH₂CH₃, (1-methoxypropan-2-yl)oxy, 2-(2-methoxyethoxy)ethoxy, SO₂Et, SPr, SOPr or SO₂Pr,
   where R² and R³ are not simultaneously hydrogen,
   with the proviso that, if R² is other than hydrogen, fluorine, chlorine or trifluoromethyl,
R³ may only have one of the following meanings:
   hydrogen, methyl or chlorine,
R⁴ is hydrogen or CH₂OCH₃,
R⁶ is hydrogen,
R⁷ is hydrogen,
R⁸ is fluorine, chlorine, bromine or CF₃,
R⁹ is hydrogen or methyl,
R¹⁰ is ethyl, isopropyl, butyl, 2,2-difluoroethyl, 2,2,2-trifluoroethyl, cyclopropyl, cyclobutyl, cyclopentyl, cyclopropylmethyl, 2-methylcyclopropyl, 2-methyl-3-oxobutan-2-yl or 3-(2-oxoazepan-1-yl)propyl,
or
R⁹ and R¹⁰ together with the nitrogen atom to which they are bonded form a piperidinyl, morpholinyl or thiomorpholinyl ring,
   and the agrochemically active salts thereof.

Furthermore preferred compounds of the formula (I) are those in which the radical R² represents a group of the formula E1, E2 or E3,

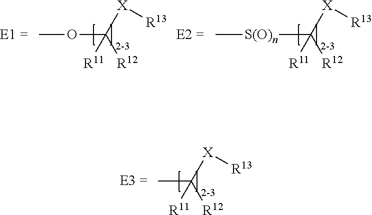

in which one or more of the symbols have one of the following meanings:
X is oxygen, NR¹⁴, sulphur, SO or SO₂,
Y is a direct bond, oxygen, NR¹⁴, sulphur, SO or SO₂,
n is 0, 1 or 2,
   where the remaining substituents have one or more of the abovementioned meanings,
   and the agrochemically active salts thereof.

Furthermore preferred compounds of the formula (I) are those in which the radical R³ represents a group of the formula E1, E2 or E3,

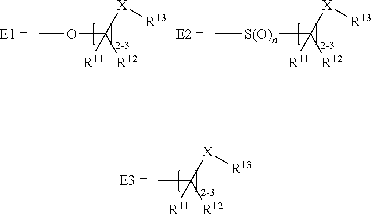

in which one or more of the symbols have one of the following meanings:
X is oxygen, NR¹⁴, sulphur, SO or SO₂,
Y is a direct bond, oxygen, NR¹⁴, sulphur, SO or SO₂,
n is 0, 1 or 2,
   where the remaining substituents have one or more of the abovementioned meanings,
   and the agrochemically active salts thereof.

Furthermore preferred compounds of the formula (I) are those in which precisely one of the radicals R² or R³ represents a group of the formula E1,

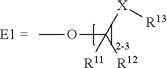

in which one or more of the symbols have one of the following meanings:
X is oxygen, NR¹⁴, sulphur, SO or SO₂,
Y is a direct bond, oxygen, NR¹⁴, sulphur, SO or SO₂,
n is 0, 1 or 2,
   where the remaining substituents have one or more of the abovementioned meanings, and the agrochemically active salts thereof.

Furthermore preferred compounds of the formula (I) are those in which precisely one of the radicals R² or R³ represents a group of the formula E2,

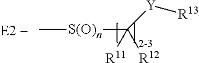

in which one or more of the symbols have one of the following meanings:
X is oxygen, NR¹⁴, sulphur, SO or SO₂,
Y is a direct bond, oxygen, NR¹⁴, sulphur, SO or SO₂,
n is 0, 1 or 2,
   where the remaining substituents have one or more of the abovementioned meanings, and the agrochemically active salts thereof.

Furthermore preferred compounds of the formula (I) are those in which precisely one of the radicals R² or R³ represents a group of the formula E3,

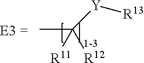

in which one or more of the symbols have one of the following meanings:
X is oxygen, Me, sulphur, SO or SO₂,
Y is a direct bond, oxygen, Me, sulphur, SO or SO₂,
n is 0, 1 or 2,
   where the remaining substituents have one or more of the abovementioned meanings,
   and the agrochemically active salts thereof.

Furthermore preferred compounds of the formula (I) are those in which
R² represents one of the following radicals:
hydrogen, O(CH₂)₂OCH₃, O(CH₂)₂OCH₂CH₃, O(CH₂)₂OCH₂CH₂CH₃, O(CH₂)₃OCH₃, O(CH₂)₃OCH₂CH₃, O(CH₂)₂O(CH₂)₂OCH₃, (1-methoxypropan-2-yl)oxy, CH₂OCH₃, CH₂OCH₂CH₃, CH₂OCH₂CH₂CH₃, 1-methoxyethyl, 1-ethoxyethyl, 1-propoxyethyl, 2,2,2-trifluoro-1-methoxyethyl, 2,2,2-trifluoro-1-ethoxyethyl, 2-methoxypropan-2-yl, 2-ethoxypropan-2-yl, phenoxymethyl, (CH₂)₂OCH₃, (CH₂)₂OCH₂CH₃, 2-methoxy-2-methylpropyl, 2-ethoxy-2-methylpropyl, SEt, SOEt, SO₂Et, SPr, SOPr, SO₂Pr, SisoPr, SOisoPr, SO₂isoPr, SBu, SOBu, SO₂Bu, SisoBu, SOisoBu, SO₂isoBu, SsecBu, SOsecBu, SO₂secBu, (2-methylprop-2-en-1-yl)sulphanyl, (2-chloroethyl)sulphonyl, (methylsulphanyl)methyl, (methylsulphinyl)methyl, (methylsulphonyl)methyl, (ethylsulphanyl)methyl, 1-(methylsulphanyl)ethyl, 1-(methylsulphinyl)ethyl, 1-(methylsulphonyl)ethyl, 1-(ethylsulphanyl)ethyl, 1-(ethylsulphinyl)ethyl, 1-(ethylsulphonyl)ethyl, 1-(methylsulphanyl)propyl,
   where the remaining substituents have one or more of the abovementioned meanings, and the agrochemically active salts thereof.

Furthermore preferred compounds of the formula (I) are those in which
R³ represents one of the following radicals:
hydrogen, methyl, fluorine, chlorine, bromine, trifluoromethyl, O(CH₂)₂OCH₃, O(CH₂)₂OCH₂CH₃, O(CH₂)₃OCH₃, O(CH₂)₃OCH₂CH₃, O(CH₂)₂O(CH₂)₂OCH₃, (1-methoxypropan-2-yl)oxy, CH₂OCH₃, CH₂OCH₂CH₃,
   where the remaining substituents have one or more of the abovementioned meanings,
   and the agrochemically active salts thereof.

Furthermore preferred compounds of the formula (I) are those in which
R⁴ is hydrogen, methyl, fluorine, chlorine, trifluoromethyl, O(CH₂)₂OCH₃, CH₂OCH₃, CH₂OCH₂CH₃,
   where the remaining substituents have one or more of the abovementioned meanings,
   and the agrochemically active salts thereof.

Furthermore preferred compounds of the formula (I) are those in which
both R¹ and R⁵ are hydrogen,
   where the remaining substituents have one or more of the abovementioned meanings,
   and the agrochemically active salts thereof.

Furthermore preferred compounds of the formula (I) are those in which
R⁶ is hydrogen,
   where the remaining substituents have one or more of the abovementioned meanings,
   and the agrochemically active salts thereof.

Furthermore preferred compounds of the formula (I) are those in which
R⁷ is hydrogen,
   where the remaining substituents have one or more of the abovementioned meanings,
   and the agrochemically active salts thereof.

Furthermore preferred compounds of the formula (I) are those in which
R⁸ is H or Me,
   where the remaining substituents have one or more of the abovementioned meanings,
   and the agrochemically active salts thereof.

Furthermore preferred compounds of the formula (I) are those in which
R⁹ is H or Me,
   where the remaining substituents have one or more of the abovementioned meanings,
   and the agrochemically active salts thereof.

Furthermore preferred compounds of the formula (I) are those in which
R¹, R⁵, R⁶ and R⁷ are hydrogen,
   where the remaining substituents have one or more of the abovementioned meanings,
   and the agrochemically active salts thereof.

Furthermore preferred compounds of the formula (I) are those in which
R¹⁰ is methyl, ethyl, propyl, isopropyl, butyl, tent-butyl, 2-methylprop-1-yl, pentyl, 2,2-dimethylprop-1-yl, 2-methylbut-1-yl, 3-methylbut-1-yl, pentan-2-yl, pentan-3-yl, prop-2-en-1-yl, prop-2-yn-1-yl, 2-fluoroethan-1-yl, 2,2-difluoroethyl, 2,2,2-trifluoroethyl, 2-chloroethan-1-yl, cyanomethyl, cyclopropyl, cyclobutyl, cyclopentyl, cyclopropylmethyl, 1-cyclopropyleth-1-yl, 2-methylcyclopropyl, 2-methyl-3-oxobutan-2-yl or 3-(2-oxoazepan-1-yl)propyl,
   where the remaining substituents have one or more of the abovementioned meanings,
   and the agrochemically active salts thereof.

Furthermore preferred compounds of the formula (I) are those in which
R¹⁰ represents 3-methylcyclobutyl or 2-ethylcyclopropyl,
   where the remaining substituents have one or more of the abovementioned meanings,
   and the agrochemically active salts thereof.

Furthermore preferred compounds of the formula (I) are those in which
R¹¹ and R¹² independently of one another represent hydrogen, methyl, ethyl, propyl, isopropyl, butyl, pentyl, hexyl, chlorine, cyclopropyl, trifluoromethyl, phenyl or benzyl, methoxymethyl,
   where the remaining substituents have one or more of the abovementioned meanings,
   and the agrochemically active salts thereof.

Furthermore preferred compounds of the formula (I) are those in which
R¹³ represents sec-butyl, isobutyl, pentan-2-yl, 2-ethoxyethyl, 2,2-dimethoxyethyl, 2,2-diethoxyethyl, 3-methoxypropyl, 3-ethoxypropyl, 2-(2-methoxyethoxy)ethyl, 2-(2-ethoxyethoxy)ethyl, trifluoromethyl, 2,2,3,3-tetrafluoropropyl,
   where the remaining substituents have one or more of the abovementioned meanings,
   and the agrochemically active salts thereof.

Furthermore preferred compounds of the formula (I) are those in which
R¹⁰ represents unsubstituted or substituted C₁-C₇-alkyl, C₂-C₇-haloalkyl, unsubstituted or substituted C₃-C₇-alkenyl or unsubstituted or substituted C₃-C₇-alkynyl,
   where the substituents in R¹⁰ independently of one another are selected from among methyl, ethyl, isopropyl, cyclopropyl, fluorine atoms, chlorine atoms and/or bromine atoms, methoxy, ethoxy, methylmercapto, ethylmercapto, cyano, hydroxyl, CF₃,
or
R¹⁰ represents C₁-C₄-alkoxy(C₁-C₄)alkyl, unsubstituted or substituted C₃-C₇-Cycloalkyl or unsubstituted or substituted C₃-C₆-cycloalkyl-(C₁-C₄)alkyl,
   where the substituents independently of one another are selected from among C₁-C₄-alkyl or C₁-C₄-haloalkyl and
   where the remaining substituents have one or more of the abovementioned meanings,
   and the agrochemically active salts thereof.

The abovementioned definitions of radicals can be combined with one another as desired.

Moreover, individual definitions may be dropped.

Examples of inorganic acids are hydrohalic acids, such as hydrogen fluoride, hydrogen chloride, hydrogen bromide and hydrogen iodide, sulphuric acid, phosphoric acid and nitric acid, and acidic salts, such as NaHSO₄ and KHSO₄. Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulphonic acids (sulphonic acids having straight-chain or branched alkyl radicals of 1 to 20 carbon atoms), arylsulphonic acids or aryldisulphonic acids (aromatic radicals, such as phenyl and naphthyl, which carry one or two sulphonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphonic acid radicals), where the alkyl and aryl radicals may carry further substituents, for example p-toluenesulphonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminium, tin and lead, and also of the first to eighth transition group, in particular chromium, manganese, iron, cobalt, nickel, copper, zinc and others. Particular preference is given to the metal ions of the elements of the fourth period. Here, the metals can be present in the various valencies that they can assume.

Optionally substituted groups may be mono- or polysubstituted, where in the case of polysubstitution the substituents may be identical or different.

In the definitions of the symbols given in the formulae above, collective terms were used which are generally representative for the following substituents:

halogen: fluorine, chlorine, bromine and iodine;

aryl: an unsubstituted or optionally substituted 5- to 15-membered partially or fully unsaturated mono-, bi- or tricyclic ring system having up to 3 ring members selected from the groups C(═O), (C═S), where at least one of the rings of the ring system is fully unsaturated, such as, for example (but not limited to) benzene, naphthalene, tetrahydronaphthalene, anthracene, indane, phenanthrene, azulene;

alkyl: saturated straight-chain or branched hydrocarbon radicals having 1 to 10 carbon atoms, such as, for example (but not limited to) methyl, ethyl, propyl, 1-methylethyl, butyl, 1-methyl-propyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, heptyl, 1-methylhexyl, octyl, 1,1-dimethylhexyl, 2-ethylhexyl, 1-ethylhexyl, nonyl, 1,2,2-trimethylhexyl, decyl,

haloalkyl: straight-chain or branched alkyl groups having 1 to 4 carbon atoms (as mentioned above), where in these groups some or all of the hydrogen atoms may be replaced by halogen atoms as mentioned above, such as, for example (but not limited to), C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoroprop-2-yl;

alkenyl: unsaturated straight-chain or branched hydrocarbon radicals having 2 to 16 carbon atoms and at least one double bond in any position, such as, for example (but not limited to), C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkynyl: straight-chain or branched hydrocarbon groups having 2 to 16 carbon atoms and at least one triple bond in any position, such as, for example (but not limited to), C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

alkoxy: saturated straight-chain or branched alkoxy radicals having 1 to 4 carbon atoms, such as, for example (but not limited to), C₁-C₄-alkoxy, such as methoxy, ethoxy, propoxy, 1-methylethoxy, butoxy, 1-methylpropoxy, 2-methylpropoxy, 1,1-dimethylethoxy;

haloalkoxy: straight-chain or branched alkoxy groups having 1 to 4 carbon atoms (as mentioned above), where some or all of the hydrogen atoms in these groups may be replaced by halogen atoms as mentioned above, such as, for example (but not limited to), C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluorethoxy, 2,2-dichloro-2-fluorethoxy, 2,2,2-trichloroethoxy, pentafluoroethoxy and 1,1,1-trifluoroprop-2-oxy;

thioalkyl: saturated straight-chain or branched alkylthio radicals having 1 to 6 carbon atoms, such as, for example (but not limited to), C₁-C₆-alkylthio, such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;

thiohaloalkyl: straight-chain or branched alkylthio groups having 1 to 6 carbon atoms (as mentioned above), where some or all of the hydrogen atoms in these groups may be replaced by halogen atoms as mentioned above, such as, for example (but not limited to) C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio;

cycloalkyl: mono-, bi- or tricyclic saturated hydrocarbon groups having 3 to 12 carbon ring members, such as, for example (but not limited to), cyclopropyl, cyclobutyl, cyclopentyl and cyclohexyl, bicyclo[1,0,1]butane, decalinyl, norbornyl;

cycloalkenyl: mono-, bi- or tricyclic non-aromatic hydrocarbon groups having 5 to 15 carbon ring members and at least one double bond, such as, for example (but not limited to) cyclopenten-1-yl, cyclohexen-1-yl, cyclohepta-1,3-dien-1-yl, norbornen-1-yl;

(alkoxy)carbonyl: an alkoxy group having 1 to 4 carbon atoms (as mentioned above) which is attached to the skeleton via a carbonyl group (—CO—);

heterocyclyl: a three- to fifteen-membered saturated or partially unsaturated heterocycle which contains one to four heteroatoms from the group consisting of oxygen, nitrogen and sulphur: mono-, bi- or tricyclic heterocycles containing, in addition to carbon ring members, one to three nitrogen atoms and/or one oxygen or sulphur atom or one or two oxygen and/or sulphur atoms; if the ring contains a plurality of oxygen atoms, these are not directly adjacent; such as, for example (but not limited to), oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazol in-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;

hetaryl: unsubstituted or optionally substituted, 5- to 15-membered partially or fully unsaturated mono-, bi- or tricyclic ring system where at least one of the rings of the ring system is fully unsaturated, comprising one to four heteroatoms from the group consisting of oxygen, nitrogen and sulphur; if the ring contains a plurality of oxygen atoms, these are not directly adjacent;

such as, for example (but not limited to),
5-membered heteroaryl which contains one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
benzo-fused 5-membered heteroaryl which contains one to three nitrogen atoms or one nitrogen atom and one oxygen or sulphur atom: 5-membered heteroaryl groups which, in addition to carbon atoms, may contain one to four nitrogen atoms or one to three nitrogen atoms and one sulphur or oxygen atom as ring members, and in which two adjacent carbon ring members or one nitrogen and one adjacent carbon ring member may be bridged by a buta-1,3-diene-1,4-diyl group in which one or two carbon atoms may be replaced by nitrogen atoms; for example benzindolyl, benzimidazolyl, benzothiazolyl, benzopyrazolyl, benzofuryl;
5-membered heteroaryl which contains one to four nitrogen atoms and is bonded via nitrogen or benzo-fused 5-membered heteroaryl which contains one to three nitrogen atoms and is bonded via nitrogen: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example 1-pyrrolyl, 1-pyrazolyl, 1,2,4-triazol-1-yl, 1-imidazolyl, 1,2,3-triazol-1-yl, 1,3,4-triazol-1-yl;

6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Not included are combinations which contradict natural laws and which the person skilled in the art would therefore have excluded based on his expert knowledge. Excluded are, for example, ring structures having three or more adjacent oxygen atoms.

The present invention furthermore relates to a process for preparing the diaminopyrimidines of the formulae (I), (Ia), (Ib) and (Ic) according to the invention, comprising at least one of steps (a) to (n) below:
(a) the reaction of 2,4-dihalopyrimidines of the formula (III) with amines of the formula (II) in the presence of a base, if appropriate in the presence of a solvent and if appropriate in the presence of a catalyst, to give compounds of the formula (V), according to the reaction scheme below (Scheme 1):

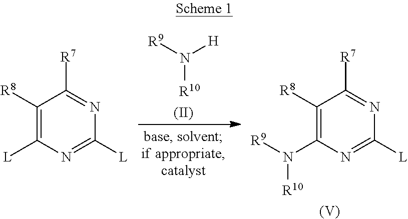

where L=F, Cl, Br, I;
(b) the reaction of compounds of the formula (V) with aromatic amines of the formula (IVa), (IVb) or (IVc), if appropriate in the presence of an acid or if appropriate in the presence of a base, if appropriate in the presence of a solvent, according to the reaction schemes below (Schemes 2, 3, 4):

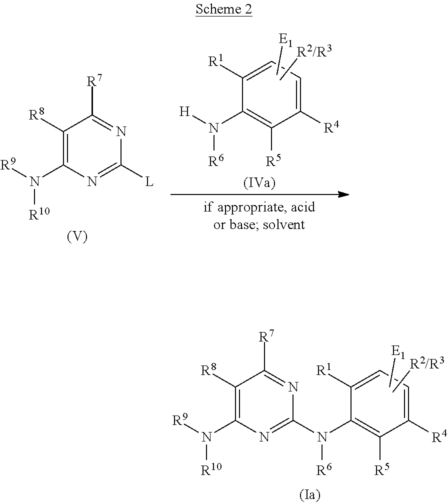

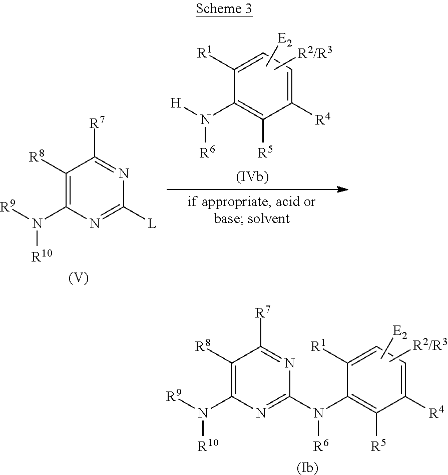

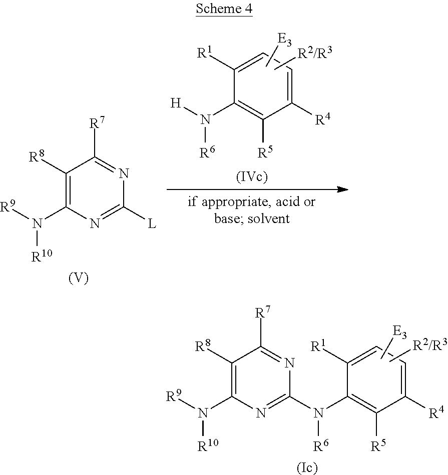

where L=F, Cl, Br, I;
(c) the oxidation of compounds of the formula (Ib-I) with a suitable oxidant to give sulphoxide compounds of the formula (Ib-II) in the presence of a solvent according to the reaction scheme below (Scheme 5):

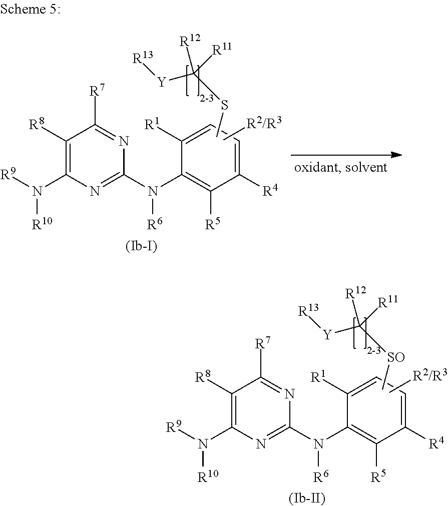

(d) the oxidation of compounds of the formula (Ib-I) with a suitable oxidant to give sulphone compounds of the formula (Ib-III) in the presence of a solvent according to the reaction scheme below (Scheme 6):

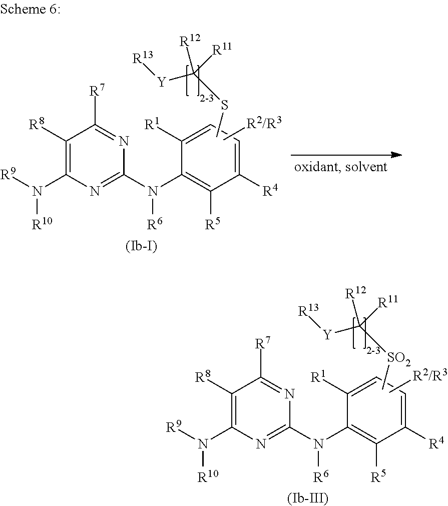

(e) the oxidation of compounds of the formula (Ic-I) with a suitable oxidant to give sulphoxide compounds of the formula (Ic-II) in the presence of a solvent according to the reaction scheme below (Scheme 7):

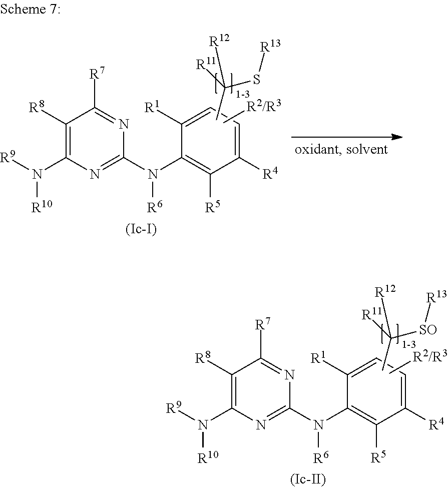

(f) the oxidation of compounds of the formula (Ic-I) with a suitable oxidant to give sulphone compounds of the formula (Ic-III) in the presence of a solvent according to the reaction scheme below (Scheme 8):

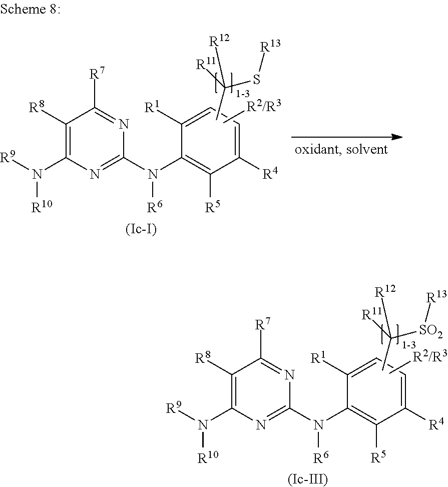

(g) the reduction of nitroaromatics of the formula (VIa), (VIb) or (Vic) to give aromatic amines of the formula (IVa), (IVb) or (IVc) by means of a suitable reducing agent, if appropriate in the presence of an acid and in the presence of a solvent, according to the reaction schemes below (Schemes 9, 10, 11):

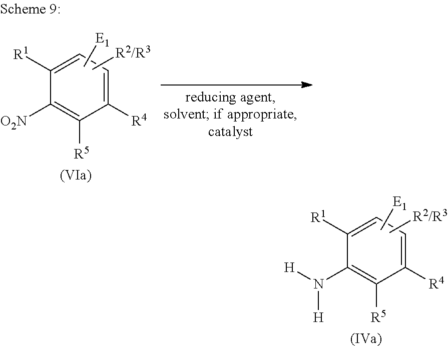

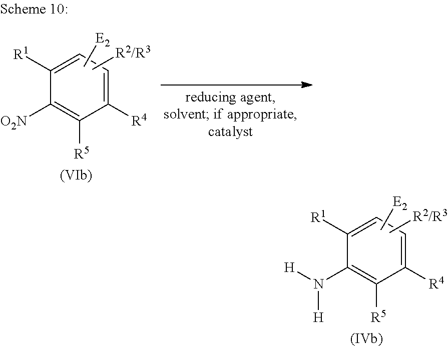

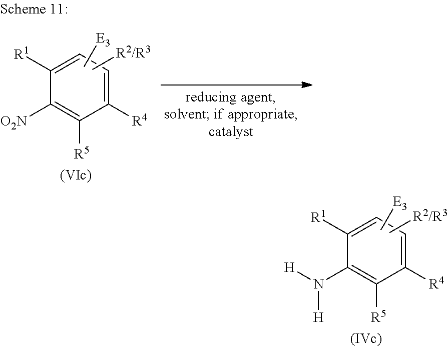

(h) the reaction of compounds of the formula (VII) with a suitable thiol to give compounds of the formula (VIc-4 if appropriate in the presence of a solvent, according to the reaction scheme below (Scheme 12):

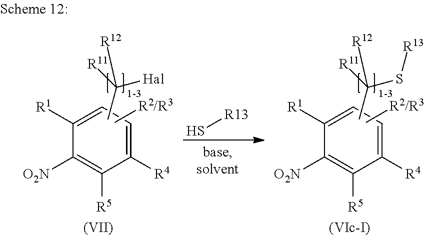

(i) the reaction of compounds of the formula (VIc-II) with a suitable chlorinating agent to give compounds of the formula (VIIa), if appropriate in the presence of a solvent, if appropriate in the presence of a suitable catalyst and if appropriate in the presence of a suitable base, according to the reaction scheme below (Scheme 13):

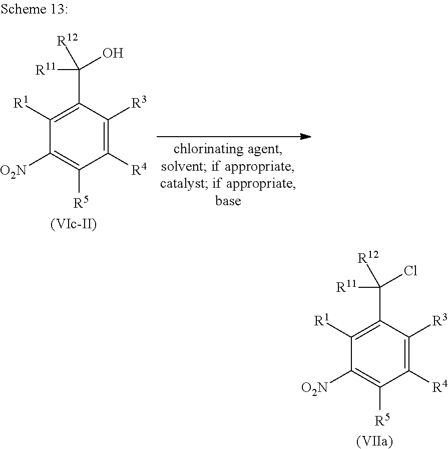

(j) the reaction of compounds of the formula (X) with amines of the formula (II) to give compounds of the formula (I) in the presence of a base, if appropriate in the presence of a solvent and if appropriate in the presence of a catalyst, according to the reaction scheme below (Scheme 14):

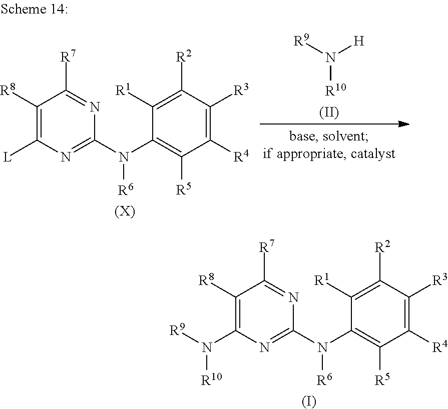

(k) the reaction of compounds of the formula (III) with anilines of the formula (IV) to give compounds of the formula (X) in the presence of a base, or in the presence of a Lewis acid, if appropriate in the presence of a solvent and if appropriate in the presence of a catalyst, according to the reaction scheme below (Scheme 15):

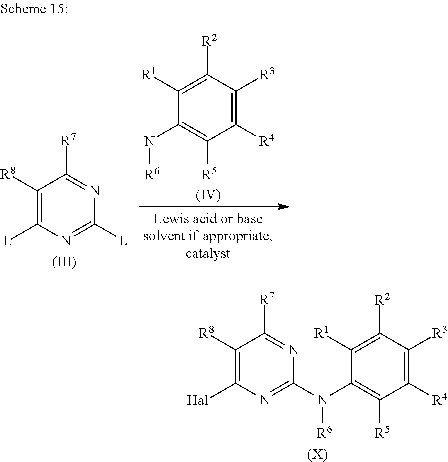

(l) the reaction of compounds of the formula (Ic-IV) with alcohols of the formula (XI) to give compounds of the formula (Ic-V) in the presence of an acid, if appropriate in the presence of a solvent and if appropriate in the presence of a catalyst, according to the reaction scheme below (Scheme 16):

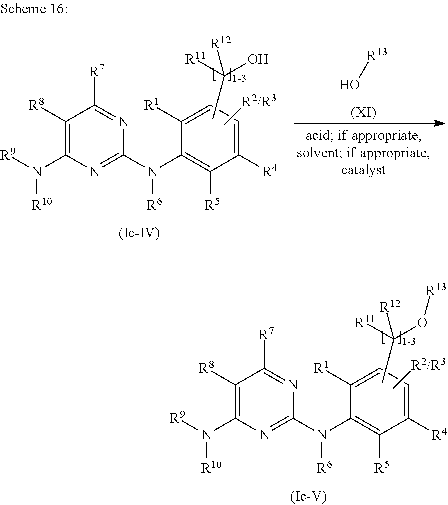

(m) the reaction of compounds of the formula (VII) with alcohols of the formula (XI) to give compounds of the formula (VIc-I), if appropriate in the presence of a solvent, according to the reaction scheme below (Scheme 17):

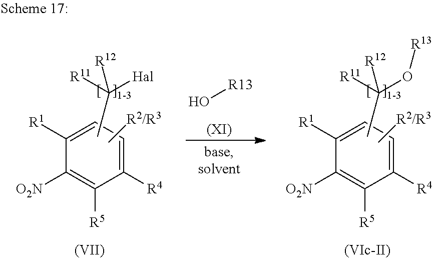

(n) the reaction of compounds of the formula (XII) with alcohols of the formula (XI) to give compounds of the formula (VIc-III) in the presence of a base, if appropriate in the presence of a solvent and if appropriate in the presence of a catalyst, according to the reaction scheme below (Scheme 18):

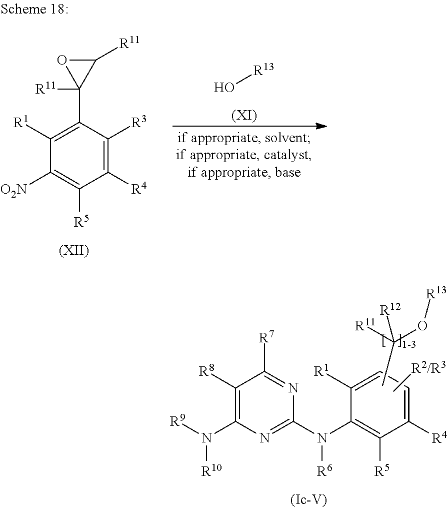

The definitions of the radicals R¹ to R¹¹ and X¹ and X² in the above schemes correspond to the abovementioned definitions, and L represents F, Cl, Br, I.

One possibility of synthesizing the intermediate (V) is shown in Scheme 1.

First, using a suitable base, an amine (II) is reacted with a 2,4-dihalopyrimidine (III) over a period of 1-24 h at a temperature of from −30° C. to +80° C. in a suitable solvent such as, for example, dioxane, THF, dimethylformamide or acetonitrile. The base used may be, for example, inorganic salts such as NaHCO₃, Na₂CO₃ or K₂CO₃, organometallic compounds such as LDA or NaHMDS or amine bases such as ethyldiisopropylamine, DBU, DBN or tri-n-butylamine. Alternatively, the reaction may also be carried out for example as described in Org. Lett. 2006, 8, 395, with the aid of a suitable transition-metal catalyst such as, for example, palladium, together with a suitable ligand such as, for example, triphenylphosphine or Xantphos.

Possibilities of synthesizing the compounds (Ia), (Ib) and (Ic) are shown in Schemes 2 to 8.

The intermediate (V) is reacted with an aromatic amine (IVa), (IVb) or (IVc) in the presence of Bronsted acids such as, for example, anhydrous hydrochloric acid, camphorsulphonic acid or p-toluenesulphonic acid in a suitable solvent such as, for example, dioxane, THF, DMSO, DME, 2-methoxyethanol, n-butanol or acetonitrile, at a temperature of from 0° C.-140° C. over a period of 1-48 h. Analogously described for example in Bioorg. Med. Chem. Lett. 2006, 16, 2689; GB2002 A1-2369359, Org. Lett. 2005, 7, 4113).

Alternatively, the reaction of (V) and (IVa), (IVb) or (IVc) to give (Ia), (Ib) and (Ic) may also be carried out with base catalysis, that is using for example carbonates such as potassium carbonate, alkoxides such as potassium tert-butoxide or hydrides such as sodium hydride, where the catalytic use of a transition metal such as, for example, palladium together with a suitable ligand such as, for example, xantphos may again be useful.

Furthermore, it is possible to carry out the reaction of (V) and (IVa), (IVb) or (IVc) to give (Ia), (Ib) and (Ic) in the absence of solvents and/or Bronsted acids and also under MW conditions (described, for example, in Bioorg. Med. Chem. Lett. 2006, 16, 108; Bioorg. Med. Chem. Lett. 2005, 15, 3881).

Finally, it is possible to obtain the compounds of the formula (Ib-II), (Ib-III), (Ic-II) or (Ic-III) by reacting compounds of the formula (Ib-I) and (Ic-I) with a suitable oxidant such as, for example, 3-chlorobenzenecarboperoxo acid, hydrogen peroxide or oxone in a suitable solvent such as, for example, dichloromethane, chloroform or acetonitrile, at a temperature of 0° C.-100° C. over a period of 1-48 hours.

Additionally, it is possible to obtain compounds of the formula (Ic-V) by reacting compounds of the formula (Ic-IV) with 1 to 50 equivalents of an alcohol of the formula (XI) in the presence of a suitable acid such as, for example, hydrochloric acid, sulphuric acid, camphorsulphonic acid or p-toluenesulphonic acid, without solvent or in a suitable solvent such as, for example, tetrahydrofuran, dioxane or acetonitrile, at a temperature of 0° C.-200° C. over a period of 1-60 hours, where the presence of 0.5 to 5 equivalents of a suitable transition metal salt such as, for example, cerium(IV) sulphate may be useful (see Scheme 16).

The alkylamino compounds of the formula (II) are either commercially available or can be prepared according to literature procedures. One method of preparing suitable cyclopropylamino compounds of the (II) type is, for example, the rearrangement reaction of suitable carboxylic acid derivatives to give the corresponding amino compounds (for example described in J. Am. Chem. Soc. 1961, 83, 3671-3678). Further methods for example for preparing cyclobutylamino compounds of the (II) type comprise the hydroboration suitable cyclobutenes followed by treatment with NH₂SO₃H (for example Tetrahedron 1970, 26, 5033-5039), the reductive aminiation of cyclobutanones (for example described in J. Org. Chem. 1964, 29, 2588-2592) and the reduction of nitro- or nitrosocyclobutanes (see, for example, J. Am. Chem. Soc. 1953, 75, 4044; Can. J. Chem. 1963, 41, 863-875) or azidocyclobutanes (for example described in Chem. Pharm. Bull. 1990, 38, 2719-2725; J. Org. Chem. 1962, 27, 1647-1650). The halogen-substituted amino compounds of the formula (II) are either commercially available or can be prepared by literature procedures. One method of producing suitable halogen-substituted amino compounds (II) is, for example, the reduction of corresponding carboxamides (for example in EP30092) or corresponding oximes or azides (for example described in Chem. Ber. 1988, 119, 2233) or nitro compounds (for example described in J. Am. Chem Soc, 1953, 75, 5006). Another possibility is the treatment of corresponding aminocarboxylic acids with SF₄ in HF (for example described in J. Org. Chem. 1962, 27, 1406). The ring opening of substituted aziridines by means of HF is described in J. Org. Chem. 1981, 46, 4938. Further methods of preparing halogen-substituted amino compounds (II) comprise the cleavage of corresponding phthalimides as described by Gabriel (for example described in DE 3429048), the aminolysis of suitable haloalkyl halides (for example described in U.S. Pat. No. 2,539,406) or the degradation of corresponding carboxylic azides (for example described in DE3611195). Amino aldehydes or amino ketones can be converted into the corresponding difluoroalkylamines by means of suitable fluorinating reagents (for example DAST; WO2008008022), while amino alcohols faun the corresponding monofluoroalkylamines (for example WO2006029115). Analogously, chloro- and bromoalkylamines can be obtained from amino alcohols by means of suitable chlorinating and brominating agents (J. Org. Chem. 2005, 70, 7364, and Org. Lett., 2004, 6, 1935, respectively).

Suitable substituted 2,4-dihalopyrimidines (III) are either commercially available or can be prepared according to literature procedures, for example starting from commercially available substituted uracils (for example R⁸═CN: J. Org. Chem. 1962, 27, 2264; J. Chem. Soc. 1955, 1834; Chem. Ber. 1909, 42, 734; R⁸═CF₃: J. Fluorine Chem. 1996, 77, 93; see also WO 2000/047539).

Some of the compounds of the formula (V) are novel and therefore also a subject matter of the present invention.

Novel compounds of the formula (Va), (Vb) and (Vc)

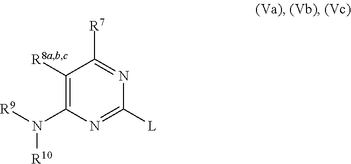

are those in which
R^{8a} represents iodine, CFH₂, CF₂H, CCl₃, cyano or Me,
R^{8b} represents CF₃;
R^{8c} represents Br;
L=F, Cl, Br or I
and
_{R}⁷, R^{8a}, R^{8b}, R^{8c}, R⁹, R¹⁰, R^{11a}, R^{11b}, and R^{11c}, as defined above, have the abovementioned general, preferred, especially preferred and very especially preferred meanings.

Some of the aromatic amino compounds of the formula (IV) and aromatic nitro compounds of the formula (VI) are commercially available or can be prepared by literature procedures.

One possibility of synthesizing the intermediate (IVa) is shown in Scheme 9.

To prepare aromatic amino compounds of the formula (IVa), suitable aromatic nitro compounds of the formula (VIa) are reacted with a suitable reducing agent such as, for example, zinc/hydrochloric acid, tin/hydrochoric acid or iron/hydrochloric acid, in a suitable solvent such as, for example, methanol, ethanol, 2-methoxyethanol or n-butanol at a temperature of 0° C.-140° C. over a period of 1-48 h. Moreover, hydrogen may also be used, using a suitable catalyst such as, for example, Raney nickel, palladium on charcoal or platinum on charcoal (for example WO 2006/128659, WO2005/49579, Macromolecules; 37; 16; 2004; 6104-6112, GB890732, CH355145, Journal of the American Chemical Society (1923), 45 2399-417, for an overview see also J. March: Advanced Organic Chemistry—Reactions, Mechanisms, and Structures, 4th Ed. (1992), Wiley, New York, page 1216ff. and literature cited therein).

Aromatic nitro compounds of the formula (Via) are either commercially available or can be prepared by literature procedures (for example WO 2006/128659, WO2005/49579, Macromolecules; 37; 16; 2004; 6104-6112)

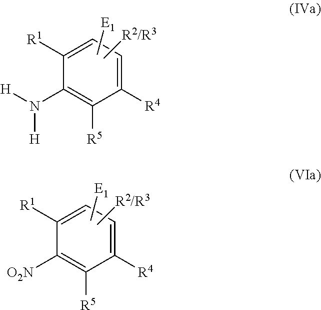

One possibility of synthesizing the intermediate (IVb) is shown in Scheme 10.

To prepare aromatic amino compounds of the formula (IVb), suitable aromatic nitro compounds of the formula (VIb) are reacted with a suitable reducing agent such as, for example, zinc/hydrochloric acid, tin/hydrochloric acid or iron/hydrochloric acid, in a suitable solvent such as, for example, methanol, ethanol, 2-methoxyethanol or n-butanol at a temperature of 0° C.-140° C. over a period of 1-48 h. Moreover, hydrogen may also be used, using a suitable catalyst such as, for example, Raney nickel, palladium on charcoal or platinum on charcoal (see for example GB890732, CH355145, Journal of the American Chemical Society (1923), 45 2399-417, GB890732, CH355145, Journal of the American Chemical Society (1923), 45 2399-417, for an overview see also J. March: Advanced Organic Chemistry—Reactions, Mechanisms, and Structures, 4th Ed. (1992), Wiley, New York, page 1216ff. and literature cited therein).

Another method of preparing suitable aromatic amino compounds (IVb) is the reaction of suitable aminobenzenethiols with organic halides to give the corresponding amino compounds (described in: Zhurnal Organicheskoi Khimii (1970), 6(4), 809-12; Bulletin de la Societe Chimique de France (1957), 1201-3).

Aminobenzenethiols and sulphanylnitrobenzenes (VIb) are either commercially available or can be prepared by literature procedures (for example Zhurnal Organicheskoi Khimii (1970), 6(4), 809-12, for an overview see also J. March: Advanced Organic Chemistry—Reactions, Mechanisms, and Structures, 4th Ed. (1992), Wiley, New York, page 407ff. and 1216ff. and literature cited therein).

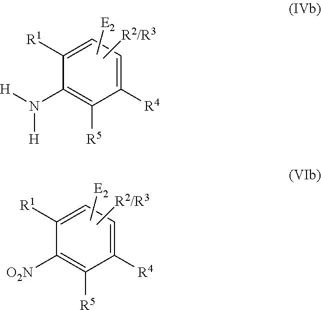

One possibility of synthesizing the compounds (IVc) is shown in Scheme 11.

To prepare aromatic amino compounds of the formula (IVc), suitable aromatic nitro compounds of the formula (VIc) are reacted with a suitable reducing agent such as, for example, zinc/hydrochloric acid, tin/hydrochoric acid or iron/hydrochloric acid, in a suitable solvent such as, for example, methanol, ethanol, 2-methoxyethanol or n-butanol at a temperature of 0° C.-140° C. over a period of 1-48 h. Moreover, hydrogen may also be used, using a suitable catalyst such as, for example, Raney nickel, palladium on charcoal or platinum on charcoal (for example GB890732, CH355145, Journal of the American Chemical Society (1923), 45 2399-417, for an overview see also J. March: Advanced Organic Chemistry—Reactions, Mechanisms, and Structures, 4th Ed. (1992), Wiley, New York, page 1216ff. and literature cited therein).

Some of the compounds of the formula (IVc) are novel and as such also subject matter of the present invention. Compounds of the formula (IVc) are described for example in the following publication and can be prepared by similar methods: Proc. Intern. Meeting Mol. Spectry. 4th, Bologna, 1959 (1962), 2, 562-576 (for an overview see also J. March: Advanced Organic Chemistry—Reactions, Mechanisms, and Structures, 4th Ed. (1992), Wiley, New York, page 407ff. and literature cited therein).

Novel compounds of the formula (IVc-I)

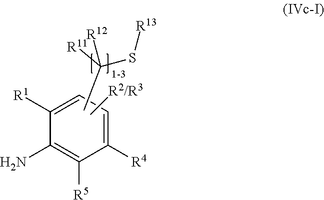

are those in which
R¹ and R⁵ represent hydrogen,
R¹¹ represents C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
and
R² to R⁴, R¹², R¹³ and R¹⁴, as defined above, have the abovementioned general, preferred, especially preferred and very especially preferred meanings.

Novel compounds of the formula (IVc-III)

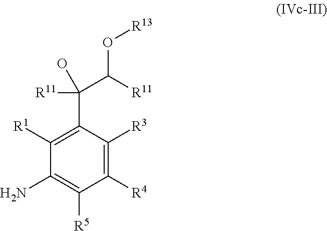

are those in which
R¹ and R⁵ represent hydrogen,
R¹¹ independently of one another represent hydrogen, C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
and
R¹³ is C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl, unsubstituted or substituted benzyl,
   where the substituents independently of one another are selected from among halogen, C₁-C₄-alkyl or C₁-C₄-haloalkyl,
and
R² to R⁴, as defined above, have the abovementioned general, preferred, especially preferred and very especially preferred meanings.

Novel compounds of the formula (IVc-IV)

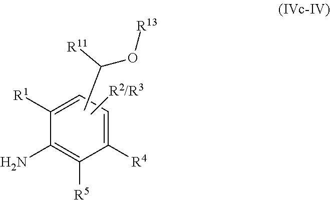

are those in which
R¹ and R⁵ represent hydrogen,
R¹¹ represents C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
and
R¹³ is C₂-C₃-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
and
R² to R⁴, as defined above, have the abovementioned general, preferred, especially preferred and very especially preferred meanings.

One possibility of synthesizing the compounds (VIc-I) is shown in Scheme 12.

To prepare aromatic nitro compounds of the formula (VIc-I), suitable aromatic nitro compounds of the formula (VII) are reacted with a suitable thiol, if appropriate in the form of the sodium or potassium salt, in a suitable solvent such as, for example, methanol, ethanol, 2-methoxyethanol or n-butanol, at a temperature of 0° C.-140° C. over a period of 1-48 h (for an overview, see also J. March: Advanced Organic Chemistry—Reactions, Mechanisms, and Structures, 4th Ed. (1992), Wiley, New York, page 407ff. or else Houben-Weyl, Methoden der Organischen Chemie, Georg-Thieme-Verlag, Stuttgart, and literature cited therein).

Some of the compounds of the formula (Vic) are novel and as such also subject matter of the present invention.

Novel compounds of the formula (VIc-I)

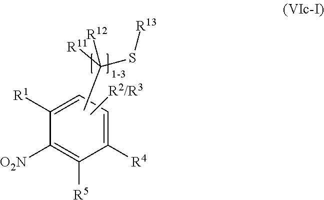

are those in which
R¹, R⁴ and R⁵ represent hydrogen,
R¹¹ represents hydrogen,
R¹² represents C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
R¹³ represents C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
R², R³ and R¹⁴, as defined above, have the abovementioned general, preferred, especially preferred and very especially preferred meanings.

Some of the aromatic nitro compounds of the formula (VIc-II) are either commercially available or can be prepared by literature procedures (for example EP 0552558, EP 0282944, EP 55616).

To prepare aromatic nitro compounds of the formula (VIc-II), suitable aromatic nitro compounds of the formula (VII) are reacted with a suitable alcohol, if appropriate in the form of the sodium or potassium salt, if appropriate in a suitable solvent such as, for example, THF, diethyl ether, dioxane or acetonitrile, at a temperature of 0° C.-140° C. over a period of 1-48 h (Scheme 19, for an overview see also J. March: Advanced Organic Chemistry—Reactions, Mechanisms, and Structures, 4th Ed. (1992), Wiley, New York, page 386ff. or else Houben-Weyl, Methoden der Organischen Chemie, Georg-Thieme-Verlag, Stuttgart, and literature cited therein).

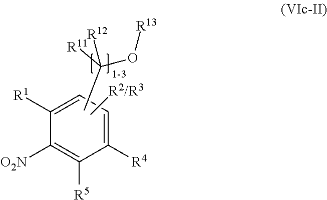

To prepare aromatic nitro compounds of the formula (VIe-III) suitable aromatic nitrophenyloxiranes of the formula (XII) are reacted with a suitable alcohol, if appropriate in the form of the sodium or potassium salt, if appropriate in a suitable solvent such as, for example, THF, diethyl ether, dioxane or acetonitrile, at a temperature of 0° C.-140° C. over a period of 1-200 hours (Scheme 18).

Novel compounds of the formula (VIc-III)

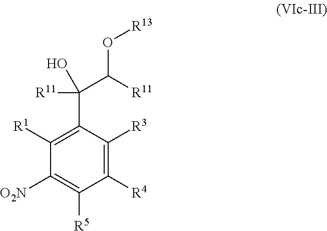

are those in which
R¹, R⁴ and R⁵ represent hydrogen,
R¹¹ independently of one another represent hydrogen, C₁-C₆-alkyl, unsubstituted or substituted
C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
R¹¹ represents C₁-C₆-alkyl, unsubstituted or substituted C₃-C₆-cycloalkyl, unsubstituted or substituted C₃-C₆-cycloalkyl(C₁-C₄)alkyl, C₂-C₃-haloalkyl, C₁-C₄-alkoxy(C₁-C₄)alkyl, unsubstituted or substituted C₂-C₄-alkenyl, unsubstituted or substituted C₂-C₄-alkynyl or unsubstituted or substituted benzyl,
   where the substituents independently of one another are selected from among halogen, C₁-C₄-alkyl or C₁-C₄-haloalkyl,
and
R² and R³, as defined above, have the abovementioned general, preferred, especially preferred and very especially preferred meanings.

Novel compounds of the formula (Vic-IV)

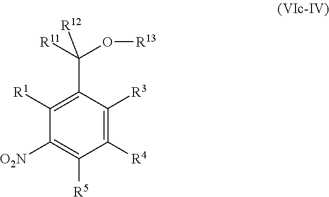

are also those in which
R¹, R⁴ and R⁵ represent hydrogen,
R¹¹ represents hydrogen,
R¹² represents C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
   where the substituents independently of one another are selected from among halogen, C₁-C₄-alkyl or C₁-C₄-haloalkyl,
R¹³ represents C₂-C₃-alkyl, unsubstituted or substituted C₃-C₆-cycloalkyl, unsubstituted or substituted C₃-C₆-cycloalkyl(C₁-C₄)alkyl, C₃-haloalkyl, C₁-C₄-alkoxy(C₁-C₄)alkyl, unsubstituted or substituted C₂-C₄-alkenyl, unsubstituted or substituted C₂-C₄-alkynyl or unsubstituted or substituted benzyl,
   where the substituents independently of one another are selected from among halogen, C₁-C₄-alkyl or C₁-C₄-haloalkyl,
R² and R³, as defined above, have the abovementioned general, preferred, especially preferred and very especially preferred meanings.

Some of the aromatic nitro compounds of the formula (VII) are commercially available or can be prepared by literature procedures (for example EP 55616, Journal of the Chemical Society, Chemical Communications (1989), (20), 1559-60, Journal of the American Chemical Society (2002), 124(46), 13690-13691, Journal of Organic Chemistry (1998), 63(17), 6023-6026, Journal of the American Chemical Society; 105; 12; 1983; 3967-3975).

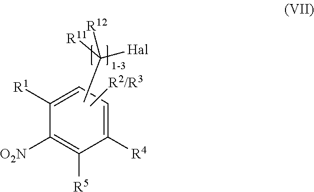

Some of the compounds of the formula (VII) are novel and as such also subject matter of the present invention.

One possibility of synthesizing the compounds (VIIa) is shown in Scheme 13.

To prepare aromatic nitro compounds (VIIa), suitable aromatic nitro compounds of the formula (VIc-II) are reacted with a suitable chlorinating agent such as, for example, concentrated hydrochloric acid, thionyl chloride, sulphuryl chloride, methanesulphonyl chloride, oxalyl chloride, phosphorus trichloride, phosphorus pentachloride or phosphoryl chloride, in a suitable solvent such as, for example, dichloromethane, DMF, chloroform or toluene, at a temperature of 0° C.-140° C. over a period of 1-48 h, if appropriate with the use of a suitable amine base such as ethyldiisopropylamine, triethylamine, DBU, DBN or tri-n-butylamine, if appropriate with the use of a suitable catalyst such as, for example, DMF (for an overview, see also J. March: Advanced Organic Chemistry—Reactions, Mechanisms, and Structures, 4th Ed. (1992), Wiley, New York, page 431ff. or else Houben-Weyl, Methoden der Organischen Chemie, Georg-Thieme-Verlag, Stuttgart and literature cited therein).

Novel compounds of the formula (VIIa)

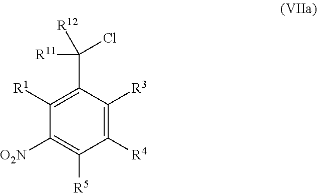

are those in which
R¹, R⁵ and R¹¹ represent hydrogen,
R¹² represents unbranched C₂-C₆-alkyl or C₃-C₆-cycloalkyl,
and
R³ and R⁴, as defined above, have the abovementioned general, preferred, especially preferred and very especially preferred meanings.

One possibility of synthesizing the compounds (I), (Ia), (Ib) and (Ic) is shown in Scheme 14.

To synthesize compounds of the formula (Ia), (Ib) and (Ic), the intermediate (X) may furthermore be reacted with alkylamino compounds of the formula (II) in the presence of bases such as, for example, carbonates such as potassium carbonate, alkoxides such as potassium tert-butoxide or hydrides such as sodium hydride in a suitable solvent such as, for example, dioxane, THF, DMSO, DME, 2-methoxyethanol, n-butanol or acetonitrile at a temperature of 0° C.-140° C. over a period of 1-48 h, where the catalytic use of a transition metal such as, for example, palladium together with a suitable ligand such as, for example, triphenylphosphine or xantphos may also be useful.

One possibility of synthesizing the compound (X) is shown in Scheme 15.

To this end, an aniline (IV) is reacted with a 2,4-dihalopyrimidine (III) over a period of 1-24 h, using a suitable Lewis acid or a suitable base, at a temperature of from −15° C. to 100° C. in a suitable inert solvent such as, for example, 1,4-dioxane, diethyl ether, THF, n-butanol, tert-butanol, dichloroethane or dichloromethane. Bases which can be employed are, for example, inorganic salts such as NaHCO₃, Na₂CO₃ or K₂CO₃, organometallic compounds such as LDA or NaHMDS, or amine bases such as ethyldiisopropylamine, DBU, DBN or tri-n-butylamine. Lewis acids which can be used are, for example (but not by limitation), halides of the metal zinc (for example ZnCl₂), magnesium, copper, tin or titanium (see, for example, US 2005/0256145 or WO 2005/023780, and literature cited therein).

The compounds of the formula (X) are novel and as such also subject matter of the present invention.

Novel compounds of the formula (X)

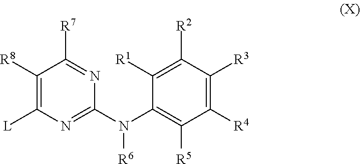

are those in which the symbols have the following meanings:
R¹ to R⁸ have the abovementioned general, preferred, especially preferred, very especially preferred and particularly preferred meanings, and
L represents fluorine, chlorine, bromine or iodine.

Alcohols of the formula (XI) are either commercially available or can be prepared by literature procedures (for example Houben-Weyl, Methoden der Organischen Chemie, Georg-Thieme-Verlag, Stuttgart, and literature cited therein).

Oxiranes of the formula (XII) are either commercially available or can be prepared by literature procedures (for example EP 305908, Houben-Weyl, Methoden der Organischen Chemie, Georg-Thieme-Verlag, Stuttgart, and literature cited therein).

In general, it is also possible to select another route for preparing the compounds (Ia), (Ib) and (Ic) according to the invention, as shown in Scheme 19.

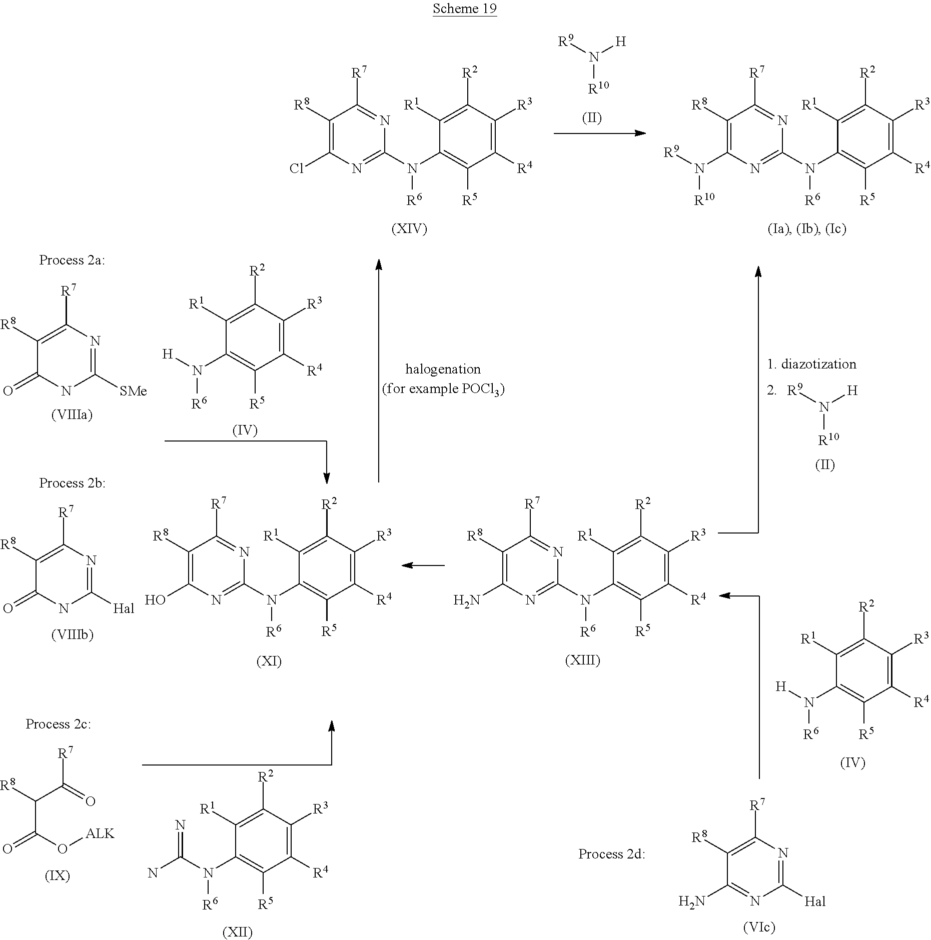

The processes according to the invention for the preparation of the compounds of the formula (Ia), (Ib) and (Ic) are preferably carried out using one or more reaction auxiliaries.

Suitable reaction auxiliaries are, if appropriate, the usual inorganic or organic base or acid acceptors. These preferably include acetates, amides, carbonates, hydrogen carbonates, hydrides, hydroxides or alkoxides of alkali metals or alkaline-earth metals, such as, for example, sodium acetate, potassium acetate, calcium acetate, lithium amide, sodium amide, potassium amide, calcium amide, sodium carbonate, potassium carbonate, calcium carbonate, sodium hydrogen carbonate, potassium hydrogen carbonate, calcium hydrogen carbonate, lithium hydride, sodium hydride, potassium hydride, calcium hydride, lithium hydroxide, sodium hydroxide, potassium hydroxide or calcium hydroxide, sodium methoxide, potassium methoxide, sodium ethoxide, potassium ethoxide, sodium n- or -i-propoxide, potassium n- or i-propoxide, sodium n-, s- or t-butoxide, potassium n-, s- or t-butoxide; furthermore also basic organic nitrogen compounds such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2,2,2]octane (DABCO), 1,5-diaza-bicyclo[4,3,0]-non-5-ene (DBN), or 1,8 diazabicyclo[5,4,0]-undec-7-ene (DBU).

The processes according to the invention are preferably carried out using one or more diluents.

Suitable diluents are virtually all inert organic solvents. These preferably include aliphatic and aromatic, optionally halogenated hydrocarbons such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers such as diethyl ether, dibutyl ether, glycol dimethyl ether and diglycol dimethyl ether, tetrahydrofuran and dioxane, ketones such as acetone, methyl ethyl ketone, methyl isopropyl ketone or methyl isobutyl ketone, esters such as methyl or ethyl acetate, nitriles such as, for example, acetonitrile or propionitrile, amides such as, for example, dimethylformamide, dimethylacetamide and N-methylpyrrolidone, and dimethyl sulphoxide, tetramethylenesulphone and hexamethylphosphoric triamide and DMPU.

In the processes according to the invention, the reaction temperatures can be varied within a substantial range. In general, the processes are carried out at temperatures between 0° C. and 250° C., preferably at temperatures between 10° C. and 185° C.

In general, the processes according to the invention are carried out under atmospheric pressure. However, it is also possible to carry out the processes under elevated or reduced pressure.

For carrying out the processes according to the invention, the starting materials required in each case are generally employed in approximately equimolar amounts: However, it is also possible to use in each case one of the components employed in a relatively large excess. Work-up in the processes according to the invention is in each case carried out by customary methods (cf. the Preparation Examples).

In general, compounds of the formula (I) can be prepared, for example, by sequential nucleophilic addition of an aliphatic amine (II) and an aromatic amine (IV) to a suitable substituted pyrimidine (III), as outlined below in Scheme 20:

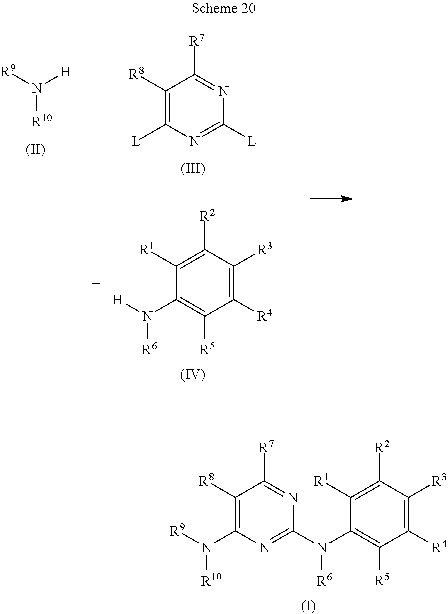

Here, L, in each case independently of one another, represent suitable leaving groups, for example a halogen atom (Hal=F, Cl, Br, I), SMe, SO₂Me, SOMe or else triflate (CF₃SO₂O: for pyrimidines known from WO 05/095386).

The synthesis of diaminopyrimidines of the formula (I) according to Scheme 20 or else by other routes has been described in the literature in many different instances (see, for example, also WO 06/021544, WO 07/072158, WO 07/003596, WO 05/016893, WO 05/013996, WO 04/056807, WO 04/014382, WO 03/030909).

The invention furthermore provides the non-medicinal use of the diaminopyrimidines according to the invention or of mixtures of these for controlling unwanted microorganisms.

The invention furthermore provides a composition for controlling unwanted microorganisms, comprising at least one diaminopyrimidine according to the present invention.

Moreover, the invention relates to a method of controlling unwanted microorganisms, characterized in that the diaminopyrimidines according to the invention are applied to the microorganims and/or their habitat.

The compounds according to the invention have strong microbicidal action and can be used for controlling unwanted microorganisms, such as fungi and bacteria, in crop protection and in the protection of materials.

The diaminopyrimidines of the formula (I) according to the invention have very good fungicidal properties and can be used in crop protection, for example for controlling Plasmodiophoromycetes, Oomycetes, Chytridiomycetes, Zygomycetes, Ascomycetes, Basidiomycetes and Deuteromycetes.

In crop protection, bactericides can be used, for example, for controlling Pseudomonadaceae, Rhizobiaceae, Enterobacteriaceae, Corynebacteriaceae and Streptomycetaceae.

The fungicidal compositions according to the invention can be used for the curative or protective control of phytopathogenic fungi. Accordingly, the invention also relates to curative and protective methods for controlling phytopathogenic fungi using the active compounds or compositions according to the invention, which are applied to the seed, the plant or plant parts, the fruit or the soil on which the plants grow.

The compositions according to the invention for controlling phytopathogenic fungi in crop protection comprise an effective, but non-phytotoxic amount of the active compounds according to the invention. “Effective, but non-phytotoxic amount” means an amount of the composition according to the invention which is sufficient to control the fungal disease of the plant in a satisfactory manner or to eradicate the fungal disease completely, while simultaneously not causing any significant symptoms of phytotoxicity. In general, this application rate may vary within a relatively wide range. It depends on a plurality of factors, for example on the fungus to be controlled, the plant, the climatic conditions and the constituents of the compositions according to the invention.

According to the invention, it is possible to treat all plants and parts of plants. Plants are to be understood here as meaning all plants and plant populations, such as wanted and unwanted wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by Plant Breeders' Rights. Parts of plants are to be understood as meaning all above-ground and below-ground parts and organs of the plants, such as shoot, leaf, flower and root, examples which may be mentioned being leaves, needles, stems, trunks, flowers, fruit bodies, fruits and seeds and also roots, tubers and rhizomes. Plant parts also include harvested material and vegetative and generative propagation material, for example seedlings, tubers, rhizomes, cuttings and seeds.

The following plants may be mentioned as plants which can be treated according to the invention: cotton, flax, grapevines, fruit, vegetables, such as Rosaceae sp. (for example pomaceous fruit, such as apples and pears, but also stone fruit, such as apricots, cherries, almonds and peaches and soft fruit such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit), Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers), Alliaceae sp. (for example leek, onion), Papilionaceae sp. (for example peas); major crop plants, such Gramineae sp. (for example maize, lawn, cereals such as wheat, rye, rice, barley, oats, millet and triticale), Asteraceae sp. (for example sunflowers), Brassicaceae sp. (for example white cabbage, red cabbage, broccoli, cauliflower, Brussels sprouts, pak choi, kohlrabi, garden radish, and also oilseed rape, mustard, horseradish and cress), Fabacae sp. (for example beans, peanuts), Papilionaceae sp. (for example soya beans), Solanaceae sp. (for example potatoes), Chenopodiaceae sp. (for example sugarbeet, fodder beet, Swiss chard, beetroot); useful plants and ornamentals in garden and forest; and also in each case genetically modified varieties of these plants. Preferably, cereal plants are treated according to the invention.

Some pathogens of fungal diseases which can be treated according to the invention may be mentioned by way of example, but not by way of limitation:

Diseases caused by powdery mildew pathogens, such as, for example, Blumeria species, such as, for example, Blumeria graminis; Podosphaera species, such as, for example, Podosphaera leucotricha; Sphaerotheca species, such as, for example, Sphaerotheca fuliginea; Uncinula species, such as, for example, Uncinula necator;

Diseases caused by rust disease pathogens, such as, for example, Gymnosporangium species, such as, for example, Gymnosporangium sabinae; Hemileia species, such as, for example, Hemileia vastatrix; Phakopsora species, such as, for example, Phakopsora pachyrhizi and Phakopsora meibomiae; Puccinia species, such as, for example, Puccinia recondita or Puccinia triticina; Uromyces species, such as, for example, Uromyces appendiculatus;

Diseases caused by pathogens from the group of the Oomycetes, such as, for example, Bremia species, such as, for example, Bremia lactucae; Peronospora species, such as, for example, Peronospora pisi or P. brassicae; Phytophthora species, such as, for example, Phytophthora infestans; Plasmopara species, such as, for example, Plasmopara viticola; Pseudoperonospora species, such as, for example, Pseudoperonospora humuli or Pseudoperonospora cubensis; Pythium species, such as, for example, Pythium ultimum;

Leaf blotch diseases and leaf wilt diseases caused, for example, by Alternaria species, such as, for example, Alternaria solani; Cercospora species, such as, for example, Cercospora beticola; Cladiosporium species, such as, for example, Cladiosporium cucumerinum; Cochliobolus species, such as, for example, Cochliobolus sativus (conidia form: Drechslera, Syn: Helminthosporium); Colletotrichum species, such as, for example, Colletotrichum lindemuthanium; Cycloconium species, such as, for example, Cycloconium oleaginum; Diaporthe species, such as, for example, Diaporthe citri; Elsinoe species, such as, for example, Elsinoe fawcettii; Gloeosporium species, such as, for example, Gloeosporium laeticolor; Glomerella species, such as, for example, Glomerella cingulata; Guignardia species, such as, for example, Guignardia bidwelli; Leptosphaeria species, such as, for example, Leptosphaeria maculans; Magnaporthe species, such as, for example, Magnaporthe grisea; Microdochium species, such as, for example, Microdochium nivale; Mycosphaerella species, such as, for example, Mycosphaerella graminicola and M. fijiensis; Phaeosphaeria species, such as, for example, Phaeosphaeria nodorum; Pyrenophora species, such as, for example, Pyrenophora teres; Ramularia species, such as, for example, Ramularia collocygni; Rhynchosporium species, such as, for example, Rhynchosporium secalis; Septoria species, such as, for example, Septoria apii; Typhula species, such as, for example, Typhula incarnata; Venturia species, such as, for example, Venturia inaequalis;

Root and stem diseases caused, for example, by Corticium species, such as, for example, Corticium graminearum; Fusarium species, such as, for example, Fusarium oxysporum; Gaeumannomyces species, such as, for example, Gaeumannomyces graminis; Rhizoctonia species, such as, for example Rhizoctonia solani; Tapesia species, such as, for example, Tapesia acuformis; Thielaviopsis species, such as, for example, Thielaviopsis basicola;

Ear and panicle diseases (including maize cobs) caused, for example, by Alternaria species, such as, for example, Alternaria spp.; Aspergillus species, such as, for example, Aspergillus flavus; Cladosporium species, such as, for example, Cladosporium cladosporioides; Claviceps species, such as, for example, Claviceps purpurea; Fusarium species, such as, for example, Fusarium culmorum; Gibberella species, such as, for example, Gibberella zeae; Monographella species, such as, for example, Monographella nivalis; Septoria species, such as, for example, Septoria nodorum;

Diseases caused by smut fungi, such as, for example, Sphacelotheca species, such as, for example, Sphacelotheca reiliana; Tilletia species, such as, for example, Tilletia caries, T. controversa; Urocystis species, such as, for example, Urocystis occulta; Ustilago species, such as, for example, Ustilago nuda; U. nuda tritici;

Fruit rot caused, for example, by Aspergillus species, such as, for example, Aspergillus flavus; Botrytis species, such as, for example, Botrytis cinerea; Penicillium species, such as, for example, Penicillium expansum and P. purpurogenum; Sclerotinia species, such as, for example, Sclerotinia sclerotiorum;

Verticillium species, such as, for example, Verticillium alboatrum;

Seed- and soil-borne rots and wilts, and also seedling diseases, caused, for example, by Fusarium species, such as, for example, Fusarium culmorum; Phytophthora species, such as, for example, Phytophthora cactorum; Pythium species, such as, for example, Pythium ultimum; Rhizoctonia species, such as, for example, Rhizoctonia solani; Sclerotium species, such as, for example, Sclerotium rolfsii;

Cankers, galls and witches' broom caused, for example, by Nectria species, such as, for example, Nectria galligena;

Wilts caused, for example, by Monilinia species, such as, for example, Monilinia laxa;

Deformations of leaves, flowers and fruits caused, for example, by Taphrina species, such as, for example, Taphrina deformans;

Degenerative diseases of woody plants caused, for example, by Esca species, such as, for example, Phaemoniella clamydospora and Phaeoacremonium aleophilum and Fomitiporia mediterranea;

Diseases of flowers and seeds caused, for example, by Botrytis species, such as, for example, Botrytis cinerea;

Diseases of plant tubers caused, for example, by Rhizoctonia species, such as, for example, Rhizoctonia solani; Helminthosporium species, such as, for example, Helminthosporium solani;

Diseases caused by bacteriopathogens, such as, for example, Xanthomonas species, such as, for example, Xanthomonas campestris pv. oryzae; Pseudomonas species, such as, for example, Pseudomonas syringae pv. lachrymans; Erwinia species, such as, for example, Erwinia amylovora.

Preference is given to controlling the following diseases of soya beans:

fungal diseases on leaves, stems, pods and seeds caused, for example, by alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

In the present case, undesired microorganisms are understood as meaning phytopathogenic fungi and bacteria. Thus, the substances according to the invention can be employed for protecting plants against attack by the abovementioned pathogens within a certain period of time after the treatment. The period of time within which their protection is effected generally extends from 1 to 10 days, preferably 1 to 7 days, after the plants have been treated with the active compounds.

The fact that the active compounds, at the concentrations required for the controlling of plant diseases, are well tolerated by plants permits the treatment of aerial plant parts, of vegetative propagation material and seed, and of the soil.

In this context, the active compounds according to the invention can be employed particularly successfully for controlling cereal diseases such as, for example, against Erysiphe species, against Puccinia and against Fusaria species, rice diseases such as, for example against Pyricularia and Rhizoctonia and diseases in viticulture, fruit production and vegetable production such as, for example, against Botrytis, Venturia, Sphaerotheca and Podosphaera species.

The active compounds according to the invention are also suitable for increasing the yield. Moreover, they display a low degree of toxicity and are well tolerated by plants.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as insecticides. If appropriate, they can also be employed as intermediates or precursors for the synthesis of other active compounds.

If appropriate, the active compounds according to the invention can also be used in certain concentrations and application rates as herbicides, for influencing plant growth and for controlling animal pests. If appropriate, they can also be employed as intermediates and precursors for the synthesis of further active compounds.

The active compounds according to the invention, in combination with good plant tolerance and favourable toxicity to warm-blooded animals and being tolerated well by the environment, are suitable for protecting plants and plant organisms, for increasing harvest yields and for improving the quality of the harvested crop. They can preferably be employed as plant protection agents. They are active against normally sensitive and resistant species and against all or some stages of development.

The treatment according to the invention of the plants and plant parts with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, drenching, drip irrigating and, in the case of propagation material, in particular in the case of seeds, furthermore as a powder for dry seed treatment, a solution for wet seed treatment, a water-soluble powder for slurry treatment, by encrusting, by coating with one or more coats, etc. It is furthermore possible to apply the active compounds by the ultra-low-volume method or to inject the active compound preparation or the active compound itself into the soil.

In addition, by the treatment according to the invention it is possible to reduce the mycotoxin content in the harvested crop and the food- and feedstuffs prepared therefrom. Particular, but not exclusive, mention may be made here of the following mycotoxins: deoxynivalenol (DON), nivalenol, 15-Ac-DON, 3-Ac-DON, T2- and HT2-toxin, fumonisins, zearalenone, moniliformin, fusarin, diacetoxyscirpenol (DAS), beauvericin, enniatin, fusaroproliferin, fusarenol, ochratoxins, patulin, ergot alkaloids and aflatoxins which can be produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec., inter alia.

In the protection of materials, the compositions or active compounds according to the invention can furthermore be employed for protecting industrial materials against attack and destruction by unwanted microorganisms, such as, for example, fungi.

In the present context, industrial materials are understood as meaning nonlife materials which have been made for use in technology. For example, industrial materials which are to be protected by active compounds according to the invention from microbial modification or destruction can be glues, sizes, paper and board, textiles, leather, timber, paints and plastic articles, cooling lubricants and other materials which are capable of being attacked or destroyed by microorganisms. Parts of production plants, for example cooling-water circuits, which can be adversely affected by the multiplication of microorganisms may also be mentioned within the materials to be protected. Industrial materials which may be mentioned with preference for the purposes of the present invention are glues, sizes, paper and board, leather, timber, paints, cooling lubricants and heat-transfer fluids, especially preferably wood. The compositions or active compounds according to the invention can prevent disadvantageous effects such as rotting, decay, discoloration, decoloration or the formation of mould.

The method according to the invention for controlling unwanted fungi can also be employed for protecting storage goods. Here, storage goods are to be understood as meaning natural substances of vegetable or animal origin or process products thereof of natural origin, for which long-term protection is desired. Storage goods of vegetable origin, such as, for example, plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The active compounds according to the invention can prevent disadvantageous effects, such as rotting, decay, discoloration, decoloration or the development of mould.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes) and against slime organisms and algae. Microorganisms of the following genera may be mentioned as examples: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Escherichia, such as Escherichia coli; Pseudomonas, such as Pseudomonas aeruginosa; Staphylococcus, such as Staphylococcus aureus.

The present invention furthermore relates to a composition for controlling unwanted microorganisms comprising at least one of the diaminopyrimidines according to the invention. These are preferably fungicidal compositions comprising auxiliaries, solvents, carriers, surfactants or extenders suitable for use in agriculture.

According to the invention, a carrier is a natural or synthetic, organic or inorganic substance with which the active compounds are mixed or bonded for better applicability, in particular for application to plants or parts of plants or seed. The carrier, which may be solid or liquid, is generally inert and should be suitable for use in agriculture.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks, such as calcite, marble, pumice, sepiolite and dolomite, and also synthetic granules of inorganic and organic meals, and granules of organic material, such as paper, sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol/POE and/or POP ethers, acid and/or POP/POE esters, alkylaryl and/or POP/POE ethers, fat and/or POP/POE adducts, POE and/or POP polyol derivatives, POE and/or POP/sorbitan or sugar adducts, alkyl or aryl sulphates, sulphonates and phosphates, or the corresponding PO ether adducts. Furthermore suitable oligo- or polymers are for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and also microencapsulations in polymeric substances.

The active compounds can be used as such, in the form of their formulations or the use forms prepared therefrom, such as ready-to-use solutions, emulsions, water- or oil-based suspensions, powders, wettable powders, pastes, soluble powders, dusts, soluble granules, granules for broadcasting, suspoemulsion concentrates, natural materials impregnated with active compound, synthetic materials impregnated with active compound, fertilizers and also microencapsulations in polymeric substances. Application is carried out in a customary manner, for example by pouring, spraying, atomizing, broadcasting, dusting, foaming, painting-on, etc. It is furthermore possible to apply the active compounds by the ultra-low-volume method or to inject the preparation of active compound or the active compound itself into the soil. It is also possible to treat the seed of the plants.

The formulations mentioned can be prepared in a manner known per se, for example by mixing the active compounds with at least one customary extender, solvent or diluent, emulsifier, dispersant and/or binder or fixative, wetting agent, water repellant, if appropriate desiccants and UV stabilizers and if appropriate colorants and pigments, antifoams, preservatives, secondary thickeners, glues, gibberellins and other processing auxiliaries.

The compositions according to the invention include not only formulations which are already ready to use and can be applied to the plant or the seed using a suitable apparatus, but also commercial concentrates which have to be diluted with water prior to use.

The active compounds according to the invention can be present as such or in their (commercial) formulations and also in the use forms prepared from these formulations as a mixture with other (known) active compounds, such as insecticides, attractants, sterilants, bactericides, acaricides, nematicides, fungicides, growth regulators, herbicides, fertilizers, safeners and/or semiochemicals.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

Liquefied gaseous extenders or carriers are liquids which are gaseous at ambient temperature and under atmospheric pressure, for example aerosol propellants, such as halogenated hydrocarbons, and also butane, propane, nitrogen and carbon dioxide.

Tackifiers, such as carboxymethylcellulose and natural and synthetic polymers in the form of powders, granules and latices, such as gum arabic, polyvinyl alcohol, polyvinyl acetate, or else natural phospholipids, such as cephalins and lecithins and synthetic phospholipids can be used in the formulations. Other possible additives are mineral and vegetable oils.

If the extender used is water, it is also possible to use, for example, organic solvents as auxiliary solvents. Suitable liquid solvents are essentially: aromatic compounds, such as xylene, toluene or alkylnaphthalenes, chlorinated aromatic compounds or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water.

The compositions according to the invention may additionally comprise further components, such as, for example, surfactants. Suitable surfactants are emulsifiers and/or foam-formers, dispersants or wetting agents having ionic or nonionic properties, or mixtures of these surfactants. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is required if one of the active compounds and/or one of the inert carriers is insoluble in water and the application is carried out in water. The proportion of surfactants is between 5 and 40 per cent by weight of the compositions according to the invention.

It is possible to use colorants such as inorganic pigments, for example iron oxide, titanium oxide, Prussian blue, and organic dyes, such as alizarin dyes, azo dyes and metal phthalocyanine dyes, and trace nutrients, such as salts of iron, manganese, boron, copper, cobalt, molybdenum and zinc.

Other possible additives are perfumes, mineral or vegetable oils, if appropriate modified, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

If appropriate, it is also possible for other additional components to be present, for example protective colloids, binders, adhesives, thickeners, thixotropic agents, penetrants, stabilizers, sequestrants, complexing agents. In general, the active compounds can be combined with any solid or liquid additive customarily used for formulation purposes.

The formulations generally comprise between 0.05 and 99% by weight, 0.01 and 98% by weight, preferably between 0.1 and 95% by weight, particularly preferably between 0.5 and 90% by weight, of active compound, very particularly preferably between 10 and 70 per cent by weight.

The formulations described above can be employed in a method according to the invention for controlling unwanted microorganisms where the diaminopyrimidines according to the invention are applied to the microorganisms and/or their habitat.

The active compounds according to the invention, as such or in their formulations, can also be used in a mixture with known fungicides, bactericides, acaricides, nematicides or insecticides, for example to broaden the activity spectrum or to prevent the development of resistance.

Suitable mixing partners are, for example, known fungicides, insecticides, acaricides, nematicides or else bactericides (see also Pesticide Manual, 13th ed.).

A mixture with other known active compounds, such as herbicides, or with fertilizers and growth regulators, safeners and/or semiochemicals is also possible.

Application is carried out in a customary manner adapted to the use forms.

The control of animal pests and/or phytopathogenic fungi which damage plants after emergence is primarily carried out by treating the soil and the above-ground parts of the plants with crop protection compositions. Owing to concerns with a view to a possible impact of the crop protection compositions on the environment and human and animal health, there are efforts to reduce the amount of active compounds applied.

The active compounds can be applied as such, in the form of their formulations and the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, painting-on, etc. It is also possible to apply the active compounds by the ultra-low-volume method or to inject the preparation of active compound or the active compound itself into the soil. It is also possible to treat the seed of the plants.

When using the active compounds according to the invention as fungicides, the application rates can be varied within a relatively wide range, depending on the type of application. The application rate of the active compounds according to the invention is
in the treatment of parts of plants, for example leaves: from 0.1 to 10 000 g/ha, preferably from 10 to 1000 g/ha, particularly preferably from 50 to 300 g/ha (when the application is by watering or dripping, it is possible to reduce the application rate even more, in particular when inert substrates such as rock wool or perlite are used);
in the treatment of seed: from 2 to 200 g per 100 kg of seed, preferably from 3 to 150 g per 100 kg of seed, particularly preferably from 2.5 to 25 g per 100 kg of seed, very particularly preferably from 2.5 to 12.5 g per 100 kg of seed;
in soil treatment: from 0.1 to 10 000 g/ha, preferably from 1 to 5000 g/ha.

These application rates are mentioned only in an exemplary manner and not limiting for the purpose of the invention.

The compounds according to the invention can also be used for protecting objects which come into contact with salt water or brackish water, such as hulls, screens, nets, buildings, moorings and signalling systems, against colonization.

The active compounds according to the invention, alone or in combination with other active compounds, can furthermore be employed as antifouling agents.

The treatment method according to the invention can be used for treating genetically modified organisms (GMOs), for example plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which is/are present in the plant (using for example, antisense technology, cosuppression technology or RNA interference—RNAi-technology), A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, the following effects, which exceed the effects which were actually to be expected, are possible: reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products.

In the present case, unwanted phytopathogenic fungi and/or microorganisms and/or viruses are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are preferably treated according to the invention include all plants with hereditary material which bestows upon these plants particularly advantageous useful properties (whether this was achieved by breeding and/or biotechnology is immaterial).

Plants and plant cultivars which are also preferably treated according to the invention are resistant against one or more biotic stresses, i.e. said plants have a better defence against animal and microbial pests, such as against nematodes, insects, mites, phytopathogenic fungi, bacteria, viruses and/or viroids.

Plants and plant cultivars which may preferably also be treated according to the invention are resistant to one or more biotic stress factors, that is to say these plants have an improved resistance to animal and microbial pests, such as nematodes, insects, mites, phytopathogenic fungi, bacteria, viruses and/or viroids.

Plants and plant cultivars which may also be treated according to the invention are those plants which are resistant to one or more abiotic stress factors. Abiotic stress conditions may include, for example, drought, cold temperature exposure, heat exposure, osmotic stress, waterlogging, increased soil salinity, increased mineral exposure, ozone exposure, high light exposure, limited availability of nitrogen nutrients, limited availability of phosphorus nutrients or shade avoidance.

Plants and plant cultivars which may also be treated according to the invention are those plants characterized by enhanced yield characteristics. Increased yield in said plants can be the result of, for example, improved plant physiology, growth and development, such as water use efficiency, water retention efficiency, improved nitrogen utilization, enhanced carbon assimilation, improved photosynthesis, increased germination efficiency and accelerated maturation. Yield can furthermore by affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod shatter and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristics of heterosis, or the hybrid effect, which results in generally higher yield, vigour, health and resistance to biotic and abiotic stress factors. Such plants are typically generated by crossing an inbred male-sterile parent line (the female parent) with another inbred male-fertile parent line (the male parent). Hybrid seed is typically harvested from the male-sterile plants and sold to growers. Male-sterile plants can sometimes (e.g. in corn) be produced by detasseling, (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described in Brassica species. However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyltransferase enzyme. Glyphosate-tolerant plants can also be obtained by selecting plants containing naturally-occurring mutations of the abovementioned genes.

Other herbicide-resistant plants are for example plants that are made tolerant to herbicides inhibiting the enzyme glutamine synthase, such as bialaphos, phosphinothricin or glufosinate. Such plants can be obtained by expressing an enzyme which detoxifies the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyse the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme. Tolerance to HPPD-inhibitors can also be obtained by transforming plants with genes encoding certain enzymes enabling the formation of homogentisate despite the inhibition of the native HPPD enzyme by the HPPD-inhibitor. Tolerance of plants to HPPD inhibitors can also be improved by transforming plants with a gene encoding an enzyme prephenate dehydrogenase in addition to a gene encoding an HPPD-tolerant enzyme.

Still further herbicide-resistant plants are plants that are made tolerant to acetolactate synthase (ALS) inhibitors. Known ALS-inhibitors include, for example, sulphonylurea, imidazolinone, triazolopyrimidines, pyrimidinyloxy(thio)benzoates, and/or sulphonylaminocarbonyltriazolinone herbicides. Different mutations in the ALS enzyme (also known as acetohydroxyacid synthase, AHAS) are known to confer tolerance to different herbicides and groups of herbicides. The production of sulphonylurea-tolerant plants and imidazolinone-tolerant plants has been described in the international publication WO 96/033270. Further sulphonylurea- and imidazolinone-tolerant plants have also been described, for example in WO 07/024782.

Other plants tolerant to imidazolinone and/or sulphonylurea can be obtained by induced mutagenesis, by selection in cell cultures in the presence of the herbicide or by mutation breeding.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:
1) an insecticidal crystal protein from Bacillus thuringiensis or an insecticidal portion thereof, such as the insecticidal crystal proteins listed online at: http://www.lifesci.sussex.ac.uk/Home/Neil_Crickmore/Bt/, or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in maize events MON863 or MON88017, or the Cry3A protein in maize event MIR604;
5) an insecticidal secreted protein from Bacillus thuringiensis or Bacillus cereus, or an insecticidal portion thereof, such as the vegetative insecticidal proteins (VIP) listed at: http://wwvv.lifesci.sussex.ac.uk/Home/Neil_Crickmore/Bt/vip.html, for example proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1a and VIP2A proteins;
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress-tolerant plants include:
a. plants which contain a transgene capable of reducing the expression and/or the activity of the poly(ADP-ribose)polymerase (PARP) gene in the plant cells or plants.
b. plants which contain a stress tolerance-enhancing transgene capable of reducing the expression and/or the activity of the PARG encoding genes of the plants or plant cells;
c. plants which contain a stress-tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage-stability of the harvested crop and/or altered properties of specific ingredients of the harvested crop such as, for example:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesized starch in wild type plant cells or plants, so that this modified starch is better suited for special applications.
2) transgenic plants which synthesize non-starch carbohydrate polymers or which synthesize non-starch carbohydrate polymers with altered properties in comparison to wild type plants without genetic modification. Examples are plants which produce polyfructose, especially of the inulin and levan type, plants which produce alpha-1,4-glucans, plants which produce alpha-1,6 branched alpha-1,4-glucans, and plants which produce alternan.
3) transgenic plants which produce hyaluronan.

Plants or plant cultivars (that can be obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics, and include:
a) plants, such as cotton plants, which contain an altered form of cellulose synthase genes,
b) plants, such as cotton plants, which contain an altered form of rsw2- or rsw3-homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the base of the fibre cell is altered, for example through downregulation of fibre-selective (3-1,3-glucanase;
f) plants, such as cotton plants, which have fibres with altered reactivity, for example through the expression of the N-acetylglucosaminetransferase gene, including nodC, and of chitin synthase genes.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Particularly useful transgenic plants which may be treated according to the invention are plants which comprise one or more genes which encode one or more toxins, are the following transgenic plants which are sold under the trade names YIELD GARD® (for example maize, cotton, soya beans), KnockOut® (for example maize), BiteGard® (for example maize), Bt-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinothricin, for example oilseed rape), IMI® (tolerance to imidazolinone) and SCS® (tolerance to sulphonylurea, for example maize). Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or a combination of transformation events, that are listed for example in the databases for various national or regional regulatory agencies (see for example http://gmoinfo.jrc.it/gmp_browse.aspx and http://www.agbios.com/dbase.php).

According to the invention, the plants listed can be treated particularly advantageously with the compounds of the general formula (I) or the active compound mixtures according to the invention. The preferred ranges indicated above for the active compounds and mixtures also apply to the treatment of these plants. Particular emphasis is given to treating the plants with the compounds and mixtures specifically indicated in the present text.

The compositions or active compounds according to the invention can thus be used to protect plants for a certain period after treatment against attack by the pathogens mentioned. The period for which protection is provided generally extends over 1 to 28 days, preferably over 1 to 14 days, particularly preferably over 1 to 10 days, very particularly preferably over 1 to 7 days, after the treatment of the plants with the active compounds, or over up to 200 days after seed treatment.

Preparation and use of the active compounds of the formula (I) according to the invention is shown in the examples below. However, the invention is not limited to these examples.

Preparation of Starting Materials of the Formula (V):

Method 1 (cf. Scheme 1)

### 2,5-Dichloro-N-cyclobutylpyrimidin-4-amine (V-1)

At −10° C., 3.39 g (24.5 mmol) of potassium carbonate are added to a solution of 3.00 g (16.4 mmol) of 2,4,5-trichloropyrimidine in 50 ml of acetonitrile. Thereafter, 1.22 g (17.2 mmol) of cyclobutylamine are added dropwise as a 20% strength acetonitrile solution. The reaction mixture is allowed to come to room temperature overnight, with stirring. The reaction mixture is stirred into 250 ml of ice-water/dilute hydrochloric acid (1:1). The mixture is extracted with ethyl acetate (2×200 ml), the combined organic phases are then washed with water (2×100 ml), and dried over MgSO₄, and the solvent is removed under reduced pressure. This gives 3.45 g (94%) of 2,5-dichloro-N-cyclobutylpyrimidin-4-amine (V-1).

logP (pH2.3): 2.62; ¹ H NMR (400 MHz, MeCN-d) δ=8.00 (s, 1H), 6.31 (br. s, 1H), 4.54-4.46 (m, 1H), 2.39-2.31 (m, 2H), 2.15-2.04 (m, 2H), 1.83-1.77 (m, 2H).

The following compounds can be prepared analogously:

5-Bromo-2-chloro-N-cyclobutylpyrimidin-4-amine (V-2); logP (pH2.3): 2.86, 1H NMR (400 MHz, DMSO-d) δ=8.20 (s, 1H), 7.52 (br. s, 1H), 4.45 (br. m, 1H), 2.24 (m, 2H), 2.17 (m, 2H), 1.69 (m, 2H).

2-Chloro-N-cyclobutyl-5-iodine-pyrimidin-4-amine (V-3) (logP (pH2.3): 3.08), ¹H NMR (400 MHz, DMSO-d) δ=8.31 (s, 1H), 7.03 (br. s, 1H), 4.47-4.03 (m, 1H), 2.28- 2.11 (m, 4H), 1.73-1.64 (m, 2H).

2,5-Dichloro-N-cyclopropylpyrimidin-4-amine (V-4); logP (pH2.3): 1.77; ¹H-NMR (400 MHz DMSO-d) 6=8.11 (s, 1H), 7.71 (br. s, 1H), 2.89-2.84 (m, 1H), 0.79-0.64 (m, 4H).

2,5-Dichloro-N-(propan-2-yl)pyrimidin-4-amine (V-5); logP (pH2.3): 2.46; ¹H NMR (400 MHz, MeCN-d) δ=7.99 (s, 1H), 5.92 (br. s, 1H), 4.31-4.23 (m, 1H), 1.25 (d, 6H)

2,5-Dichloro-N-propylpyrimidin-4-amine (V-6): logP (pH2.3): 2.42; ¹H NMR (400 MHz, DMSO-d) δ=8.14 (s, 1H), 7.94 (br. s, 1H), 3.30 (t, 2H), 1.58-1.53 (m, 2H), 0.87 (t, 3H).

2-Chloro-N-cyclobutyl-5-fluoropyrimidin-4-amine (V-7); logP (pH2.3): 2.17; ¹H NMR (400 MHz, MeCN-d) δ=7.84 (d, 1H), 6.37 (br. s, 1H), 4.54-4.43 (m, 1H), 2.40-2.30 (m, 2H), 2.12-2.04 (m, 2H), 1.91-1.71 (m, 2H).

4-(2,5-Dichloropyrimidin-4-yl)morpholine (V-8); logP (pH2.3): 1.99; ¹H NMR (400 MHz, DMSO-d) δ=8.27 (s, 1H), 3.76-3.69 (m, 8H).

4-(2,5-Dichloropyrimidin-4-yl)thiomorpholine (V-9); logP (pH2.3): 2.84; ¹H NMR (400 MHz, DMSO-d) δ=8.27 (s, 1H), 3.99-3.96 (m, 4H), 2.76-2.73 (m, 4H).

2,5-Dichloro-4-(pyrrolidin-1-yl)pyrimidine (V-10); logP (pH2.3): 2.78; ¹H NMR (400 MHz, DMSO-d) δ=8.09 (s, 1H), 3.75-3.71 (m, 4H), 1.92-1.86 (m, 4H).

2,5-Dichloro-N-methyl-N-(propan-2-yl)pyrimidin-4-amine (V-11); logP (pH2.3): 3.16; ¹H NMR (400 MHz, MeCN-d) δ=8.04 (s, 1H), 4.82-4.76 (m, 1H), 3.03 (s., 3H), 1.22 (d, 6H).

[(2,5-Dichloropyrimidin-4-yl)amino]acetonitrile (V-12); logP (pH2.3): 1.27; ¹H NMR (400 MHz, MeCN-d) δ=8.16 (s, 1H), 6.70 (br. s, 1H), 4.35 (d, 2H).

2,5-Dichloro-4-(piperidin-1-yl)pyrimidine (V-13); logP (pH2.3): 3.52; ¹H NMR (400 MHz, DMSO-d) δ=8.20 (s, 1H), 3.71-3.69 (m, 4H), 1.67-1.59 (m, 6H).

2,5-Dichloro-N-ethyl-N-methylpyrimidin-4-amine (V-14); logP (pH2.3): 2.68; ¹H NMR (400 MHz, DMSO-d) δ=8.14 (s, 1H), 3.67 (q, 2H), 3.18 (s, 3H), 1.19 (t, 3H).

5-Bromo-2-chloro-N-cyclopropylpyrimidin-4-amine (V-15); logP (pH2.3): 1.97; ¹H NMR (400 MHz, MeCN-d) δ=8.12 (s, 1H), 6.17 (br. s, 1H), 2.87-2.80 (m, 1H), 0.85-0.79 (m, 2H) 0.66-0.62 (m, 2H).

2,5-Dichloro-N-(2,2,2-trifluoroethyl)pyrimidin-4-amine (V-16); logP (pH2.3): 2.26; ¹H NMR (400 MHz, DMSO-d) δ=8.29 (s, 1H), 8.25 (br. s, 1H), 4.24-4.15 (m, 2H).

2,5-Dichloro-N-(2,2-difluoroethyl)pyrimidin-4-amine (V-17); logP (pH2.3): 1.96; ¹H NMR (400 MHz, MeCN-d) δ=8.10 (s, 1H), 6.47 (br. s, 1H), 6.02 (tt, 1H), 3.86 (m, 2H).

2,5-Dichloro-N-ethylpyrimidin-4-amine (V-18) (logP (pH2.3): 1.93); ¹H NMR (400 MHz, MeCN-d) δ=7.99 (s, 1H), 6.23 (br. s, 1H), 3.48 (q, 2H), 1.20 (t, 3 H).

2,5-Dichloro-N-cyclopropyl-N-methylpyrimidin-4-amine (V-19); logP (pH2.3): 2.82; ¹H NMR (400 MHz, MeCN-d) δ=8.09 (s, 1H), 3.15-3.12 (m, 1H), 3.11 (s, 3H), 0.87-0.82 (m, 2H), 0.72-0.70 (m, 2H).

2,5-Dichloro-N-(3-methylcyclobutyl)pyrimidin-4-amine (V-23) (main isomer: logP (pH2.3): 3.20; ¹H NMR (400 MHz, DMSO-d6) δ=8.10 (s, 1H), 7.72 (s, 1H), 4.25-4.31 (m, 1H), 2.29-2.35 (m, 3H), 1.92-1.99 (m, 2H), 1.06 (d, 3H).

5-Bromo-2-chloro-N-(3-methylcyclobutyl)pyrimidin-4-amine (V-24) (main isomer: logP (pH2.3): 3.47; ¹H NMR (400 MHz, DMSO-d6) δ=8.19 (s, 1H), 7.46 (s, 1H), 4.25-4.30 (m, 1H), 2.31-2.35 (m, 3H), 1.93-1.99 (m, 2H), 1.05 (d, 3H).

2,5-Dichloro-N-(cyclopentyl)pyrimidin-4-amine (V-25), logP (pH2.3): 3.16; ¹H NMR (400 MHz, DMSO-d) δ=8.11-8.09 (d, 1H), 7.36 (d, 1H), 4.36-4.28 (m, 1H), 1.98-1.93 (m, 2H), 1.73-1.67 (m, 2H), 1.64-1.53 (m, 4H).

2,5-Dichloro-N-(2-methylcyclopropyl)pyrimidin-4-amine (V-26) (logP (pH2.3): 2.53; ¹H NMR (400 MHz, DMSO-d6, main isomer) δ=8.10 (s, 1H), 7.49 (s, 1H), 2.48-2.49 (m, 1H), 1.09 (d, 3H), 0.96-1.02 (m, 1H), 0.81-0.85 (m, 1H), 0.53-0.58 (m, 1H).

5-Bromo-2-chloro-N-(2-methylcyclopropyl)pyrimidin-4-amine (V-27) (logP (pH2.3): 2.68; ¹H NMR (400 MHz, DMSO-d6, main isomer) δ=8.19 (s, 1H), 7.71 (s, 1H), 1.09 (d, 3H), 0.90-1.06 (m, 2H), 0.81-0.86 (m, 1H), 0.53-0.58 (m, 1H).

2-Chloro-N-(2-methylcyclopropyl)-5-(trifluoromethyl)pyrimidin-4-amine (V-28) (logP (pH2.3): 3.02; ¹H NMR (600 MHz, DMSO-d6, main isomer) δ=8.39 (s, 1H), 8.00 (s, 1H), 1.10 (d, 3H), 0.84-1.08 (m, 3H), 0.57-0.66 (m, 1H).

2,5-Dichloro-N-(2-ethylcyclopropyl)pyrimidin-4-amine (V-29) (logP (pH2.3): 3.10; ¹H NMR (400 MHz, DMSO-d6, main isomer) δ=8.10 (s, 1H), 7.70 (s, 1H), 2.48-2.56 (m, 1H), 1.25-1.40 (m, 2H), 1.00-1.04 (q, 2H), 0.85-0.77 (m, 1H), 0.82-0.84 (m, 1H), 0.56-0.60 (m, 1H).

Method 2 (cf. Scheme 1)

### 2-Chloro-N-cyclobutyl-5-trifluoromethylpyrimidin-4-amine (V-20)

A mixture of 8.07 g (37.2 mmol) of 2,4-dichloro-5-trifluoropyrimidine and 12.8 g (92.9 mmol) of potassium carbonate in 150 ml of acetonitrile is warmed to 50° C. Then, 4.00 g (37.2 mmol) of cyclobutylamine hydrochloroide are added and stirring is continued for 2 h. When cold, the reaction mixture is stirred into 500 ml of ice-water and extracted with ethyl acetate (3×200 ml). The combined organic phases are separated, washed with water (2×250 ml), dried over MgSO₄ and freed from solvent under reduced pressure. The crude product is purified by column chromatography over silica gel (cyclohexane/ethyl acetate). This gives 4.00 g (41%) of 2-chloro-N-cyclobutyl-5-trifluoromethylpyrimidin-4-amine. logP (pH2.3): 3.20; ¹H NMR (400 MHz, MeCN-d) δ=8.27 (s, 1H), 6.19 (br. s, 1H), 4.64-4.56 (m, 1H), 2.40-2.32 (m, 2H), 2.14-2.04 (m, 2H), 1.82-1.74 (m, 2H).

The following compounds can be prepared analogously:

2-Chloro-N-cyclopropyl-5-(trifluoromethyl)pyrimidin-4-amine (V-21); logP (pH2.3): 2.38; ¹H NMR (400 MHz, MeCN-d) δ=8.28 (s, 1H), 6.34 (br. s, 1H), 2.91-2.86 (m, 1H), 0.85-0.80 (m, 2H), 0.66-0.62 (m, 2H).

2-Chloro-N-(cyclopropylmethyl)-5-(trifluoromethyl)pyrimidin-4-amine (V-22); logP (pH2.3): 2.98; ¹H NMR (400 MHz, DMSO-d6) δ=8.05 (s, 1H), 7.51 (br. s., 1H), 3.02 (t, 2H), 0.79-0.89 (m, 1H), 0.11-0.17 (m, 2H), −0.03-0.03 (m, 2H); M+H=252.0.

Preparation of Compounds of the Formula (Ia) (cf. Scheme 2)

### 5-Bromo-N⁴-cyclopropyl-N²-{4-[(1-methoxypropan-2-yl)oxy]phenyl}pyrimidine-2,4-diamine (Compound 1)

A mixture of 150 mg (0.6 mmol) of 5-bromo-2-chloro-N-cyclopropylpyrimidin-4-amine, 130 mg (0.72 mmol) of 4-[(1-methoxypropan-2-ypoxy]aniline and 88 mg (0.51 mmol) of 4-toluenesulphonic acid in 5 ml of dioxane is stirred for 18 h at 105° C. When cold, the reaction mixture is concentrated under reduced pressure and the residue is taken up in 50 ml of ethyl acetate. The organic phase is washed with 10 ml of saturated aqueous NaHCO₃ and then with 10 ml of water, dried over MgSO₄ and freed from solvent under reduced pressure. The crude product is then purified by column chromatography over silica gel, with tert-butyl methyl ether as the mobile phase. This gives 130 mg of the desired product; logP (pH2.3): 1.83; ¹H NMR (400 MHz, DMSO-d) δ=885 (s, 1H), 7.94 (s, 1H), 7.68-7.66 (m, 2H), 6.85-6.80 (m, 2H), 6.68-6.65 (m, 1H), 4.44-4.41 (m, 1H), 3.49-3.39 (m, 2H), 3.20 (s, 3H), 2.82-2.80 (m, 1H), 2.25-2.18 (m, 2H), 1.05-1.00 (m 1H), 0.88-0.84 (m 2H), 0.78-0.72 (m, 2H), 0.65-0.61 (m, 2H).

Preparation of Compounds of the Formula (Ib)

Method 1 (cf. Scheme 3)

### 5-Chloro-N⁴-(propan-2-yl)-N²-[3-(propylsulphanyl)phenyl]pyrimidine-2,4-diamine (Compound 31)

At room temperature, 101 mg (0.59 mmol) of 4-toluenesulphonic acid are added to a solution of 303 mg (1.47 mmol) of 2,5-dichloro-N-(propan-2-yl)pyrimidin-4-amine and 295 mg (1.76 mmol) of 3-(propylsulphanyl)aniline in 25 ml of toluene, and the mixture is heated at 110° C. After 16 hours, the reaction mixture is freed from solvent under reduced pressure. The crude product is then purified by column chromatography over silica gel, with tert-butyl methyl ether as the mobile phase. This gives 300 mg of the desired product (logP (pH2.3): 3.27).

¹H NMR (400 MHz, DMSO-d) δ=8.98 (s, br., 1H), 7.91 (s, 1H), 7.83 (s, br., 1H), 7.48-7.45 (d, 1H), 7.15 (t, 1H), 6.87-6.84 (d, 1H), 6.49-6.47 (d, br., 1H), 4.38-4.29 (m, 1H), 2.98 (t, 2H), 2.98 (t, 2H), 1.66-1.57 (m, 2H), 1.26-1.24 (d, 6H), 0.96 (t, 3H).

Method 2 (cf. Schemes 5 and 6)

### 5-Chloro-N⁴-cyclobutyl-N²-[3-(ethylsulphinyl)phenyl]pyrimidine-2,4-diamine (Compound 28) and 5-chloro-N⁴-cyclobutyl-N²-[3-(ethylsulphonyl)phenyl]pyrimidine-2,4-diamine (Compound 27)

At 20° C., 552 mg (2.24 mmol) of 70% strength 3-chlorobenzenecarboperoxo acid are added portionwise to a solution of 500 mg (1.49 mmol) of 5-chloro-N⁴-cyclobutyl-N²-[3-(ethylsulphanyl)-phenyl]pyrimidine-2,4-diamine in 20 ml of dichloromethane, and the mixture is stirred for 16 hours at the same temperature. After 16 hours, the reaction mixture is stirred with 20 ml of 2 M sodium hydroxide solution and, if tested positive for peroxide, with 10 ml of aqueous sodium bisulphite solution. Thereafter, the organic phase is separated off, washed with 20 ml of water, dried over MgSO₄ and freed from solvent under reduced pressure. The crude product is then subjected to fractional chromatography over silica gel, with tert-butyl methyl ether as the mobile phase. This gives 120 mg of 5-chloro-N⁴-cyclobutyl-N²-[3-(ethylsulphinyl)phenyl]pyrimidine-2,4-diamine (logP (pH2.3): 1.76; ¹H NMR (400 MHz, DMSO-d) 6=9.30 (s, br., 111), 8.29 (s, br., 1H), 7.94 (s, 1H), 7.68-7.65 (d, 1H), 7.41 (t, 1H), 7.13-7.11 (d, 1H), 7.04-7.02 (d, br., 1H), 4.69-4.59 (m, 1H), 2.98-2.93 (m, 1H), 2.77-2.72 (m, 1H), 2.34-2.28 (m, 2H), 2.16-2.10 (m, 2H, 1.74-1.69 (m, 2H), 1.09 (t, 3H)) and 130 mg of 5-chloro-N⁴-cyclobutyl-N²43-(ethylsulphonyl)phenyl]pyrimidine-2,4-diamine (logP (pH2.3): 2.21).

Method 3 (cf. Scheme 15)

### N⁴-Cyclobutyl-N²-[3-(propylsulphonyl)phenyl]-5-(trifluoromethyl)pyrimidine-2,4-diamine (Compound 34)

At 20° C., a solution of 0.16 g (0.41 mmol) of 4-chloro-N-[3-(propylsulphonyl)phenyl]-5-(trifluoromethyl)pyrimidin-2-amine in 5 ml of acetonitrile is treated with 68 mg (0.49 mmol) of potassium carbonate and 35 mg (0.49 mmol) of cyclobutylamine. The reaction mixture is stirred for 16 hours at 20° C. Then, the solvent is removed under reduced pressure, the residue is stirred with water and the solid faulted is filtered off with suction. This gives 150 mg of N⁴-cyclobutyl-N²-[3-(propylsulphonyl)phenyl]-5-(trifluoromethyl)pyrimidine-2,4-diamine, (logP (pH2.3): 3.46).

¹H NMR (400 MHz, DMSO-d) δ=9.82 (s, br., 1H), 8.62 (s, br., 1H), 8.22 (s, 1H), 7.90-7.84 (d, 1H), 7.55 (t, 1H), 7.48-7.44 (d, 1H), 6.82-6.81 (d, br., 1H), 4.80-4.70 (m, 1H), 3.25-3.19 (m, 2H), 2.35-2.25 (m, 2H), 2.17-2.12 (m, 2H), 1.77-1.58 (m, 4H), 0.94 (t, 3H).

Preparation of Compounds of the Formula (Ic)

Method 1 (cf. Scheme 4)

### 5-Chloro-N⁴-cyclopropyl-N²-{3-[1-(methylsulphanyl)propyl]phenyl}pyrimidine-2,4-diamine (Compound 61)

A mixture of 258 mg (1.26 mmol) of 2,5-dichloro-N-cyclopropylpyrimidin-4-amine, 229 mg (1.26 mmol) of 3-[1-(methylsulphanyl)propyl]aniline and 87 mg (0.51 mmol) of 4-toluenesulphonic acid in 20 ml of toluene is stirred for 16 h at 110° C. After 16 hours, the reaction mixture is freed from solvent under reduced pressure. The crude product is then purified by column chromatography over silica gel, with tert-butyl methyl ether as the mobile phase. This gives 300 mg of the desired product (logP (pH2.3): 2.59).

¹H NMR (400 MHz, DMSO-d) δ=8.99 (s, br., 1H), 7.90 (s, 1H), 7.82 (s, br., 1H), 7.67-7.65 (m, 1H), 7.17 (t, 1H), 6.95 (s, br., 1H), 6.83-6.82 (d, 1H), 3.57-3.54 (m, 1H), 1.89-1.74 (m, 5H), 0.86 (t, 3H), 0.80-0.72 (m, 2H), 0.69-0.65 (m, 2H).

Method 2 (cf. Schemes 7 and 8)

### N⁴-Cyclobutyl-N²-{3-[1-(ethylsulphinyl)ethyl]phenyl}-5-(trifluoromethyl)pyrimidine-2,4-diamine (Compound 43) and

### N⁴-cyclobutyl-N²-{3-[1-(ethylsulphonyl)ethyl]phenyl}-5-(trifluoromethyl)pyrimidine-2,4-diamine (Compound 40)

At 20° C., 840 mg (3.41 mmol) of 70% strength 3-chlorobenzenecarboperoxo acid is added portionwise to a solution of 900 mg (2.27 mmol) of N⁴-cyclobutyl-N²-{3-[1-(ethylsulphanyl)ethyl]phenyl}-5-(trifluoromethyl)pyrimidine-2,4-diamine in 20 ml of dichloromethane and the mixture is stirred for 16 hours at the same temperature. After 16 hours, the reaction mixture is stirred with 20 ml of 2 M sodium hydroxide solution and, if tested positive for peroxide, with 10 ml of aqueous sodium bisulphite solution. Thereafter, the organic phase is separated off, washed with 20 ml of water, dried over MgSO₄ and freed from solvent under reduced pressure. The crude product is then subjected to fractional chromatography over silica gel, with tert-butyl methyl ether as the mobile phase. This gives 200 mg of N⁴-Cyclobutyl-N²-{3-[1-(ethylsulphinyl)ethyl]phenyl}-5-(trifluoromethyl)pyrimidine-2,4-diamine (logP (pH2.3): 2.55),

¹H NMR (400 MHz, DMSO-d) δ=9.43 (s, br., 1H), 8.18 (s, 1H), 7.83 (s, br., 1H), 7.60-7.57 (d, 1H), 7.28 (t, 1H), 6.96-6.94 (d, 1H), 6.73-6.71 (d, br., 1H), 4.77-4.66 (m, 1H), 3.93-3.84 (m, 1H), 2.60-2.40 (m), 2.33-2.25 (m, 2H), 2.20-2.10 (m, 2H), 1.76-1.65 (m, 2H), 1.61-1.53 (m, 3H), 1.20-1.10 (m, 4H),

and 200 mg of N⁴-cyclobutyl-N²-{3-[1-(ethylsulphonyl)ethyl]phenyl}-5-(trifluoromethyl)pyrimidine-2,4-diamine; logP (pH2.3): 2.89;

¹H NMR (400 MHz, DMSO-d) δ=9.44 (s, br., 1H), 8.17 (s, 1H), 7.90 (s, br., 1H), 7.70-7.67 (d, 1H), 7.30 (t, 1H), 7.09-7.07 (d, 1H), 6.63-6.60 (d, br., 1H), 4.76-4.68 (m, 1H), 2.99-2.84 (m, 2H), 2.33-2.23 (m, 2H), 2.20-2.10 (m, 2H), 1.75-1.63 (m, 5H), 1.16 (t, 3H).

Method 3 (cf. Scheme 16)

### 5-Chloro-N4-cyclopropyl-N2-{3-[1-(3-methoxypropoxy)ethyl]phenyl}pyrimidine-2,4-diamine (Compound 256)

A mixture of 1.0 g (3.0 mmol) of 1-(3-{[5-chloro-4-(cyclopropylamino)pyrimidin-2-yl]amino}-phenyl)ethanol, 1.33 g (3.28 mmol) of cerium (IV) sulphate (as the tetrahydrate) and 3.10 g (16.4 mmol) of 4-toluenesulphonic acid is stirred for 20 h at 150° C. in 20 ml of 3-methoxypropan-1-ol. When cold, the reaction mixture is concentrated under reduced pressure and the residue is neutralized with aqueous bicarbonate solution. The mixture is extracted with dichloromethane. Thereafter, the organic phase is filtered with suction through Celite 545, dried using sodium sulphate and freed from solvent under reduced pressure. Thereafter, the crude product is purified by column chromatography over silica gel, with tert-butyl methyl ether/petroleum ether 1:1 as the mobile phase. This gives 200 mg of the desired product; logP (pH2.3): 2.20;

¹H NMR (400 MHz, DMSO-d) δ=9.24 (s, br., 1H), 7.93 (s, 2H), 7.67-7.65 (d, 1H), 7.23-7.18 (m, 2H), 6.83-6.81 (d, 1H), 4.33-4.28 (m, 1H), 3.37-3.24 (m), 3.17 (s, 3H), 2.90-2.86 (m, 1H), 1.71-1.65 (m, 2H), 1.31-1.30 (d, 3H), 0.82-0.78 (m, 2H), 0.68-0.66 (m, 2H).

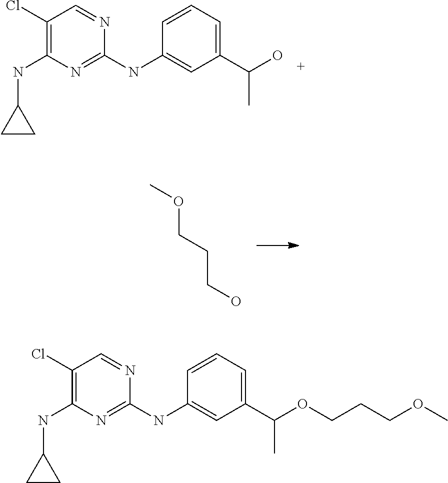

Preparation of starting compounds of the formula (IVc-I):

Method 1 (cf. Scheme 11)

### 3-[1-(Methylsulphanyl)propyl]aniline (IVc-I-1)

A solution of 5.4 g (26 mmol) of 1-[1-(methylsulphanyl)propyl]-3-nitrobenzene in a mixture of 20 ml of methanol and 20 ml of concentrated hydrochloric acid is treated carefully with 12.7 g (107 mmol) of tin and the reaction mixture is stirred for 1 hour at 40° C. When cold, it is filtered through Celite, and the mother liquor is concentrated under reduced pressure. The residue is taken up in 50 ml of dichloromethane and the mixture is neutralized with 2 M aqueous sodium hydroxide solution. After the organic phase has been separated off, it is washed with 10 ml of water, dried over MgSO₄ and freed from solvent under reduced pressure. Thereafter, the crude product is purified by column chromatography over silica gel, using tert-butyl methyl ether as the mobile phase. This gives 2.5 g of the desired product (logP (pH2.3): 1.49);

¹H NMR (400 MHz, DMSO-d) δ=6.94 (t, 1H), 6.53-6.52 (m, 1H), 6.45-6.42 (m, 2H), 4.82 (s, br., 2H), 3.47-3.44 (m, 1H), 1.85 (s, 3H), 1.83-1.73 (m, 2H), 0.85 (t, 3H).

The following compounds can be prepared analogously:

3-[1-(Methylsulphanypethyl]aniline (IVc-I-2); logP (pH2.3): 1.03;

¹H NMR (400 MHz, DMSO-d) δ=6.94 (t, 1H), 6.56-6.55 (m, 1H), 6.48-6.46 (d, 1H), 6.45-6.42 (m, 1H), 4.82 (s, br., 2H), 3.75-3.70 (q, 1H), 1.88 (s, 3H), 1.46-1.44 (d, 3H).

3-[1-(Ethylsulphanyl)propyl]aniline (IVc-I-3); logP (pH2.3): 1.94;

¹H NMR (400 MHz, DMSO-d) δ=6.93 (t, 1H), 6.54-6.53 (m, 1H), 6.45-6.41 (m, 2H), 4.80 (s, br., 2H), 3.59-3.55 (m, 1H), 2.34-2.25 (m, 2H), 1.82-1.68 (m, 2H), 1.09 (t, 3H), 0.84 (t, 3H).

Method 2 (cf. Scheme 11)

### 3-[1-(Ethylsulphanyl)ethyl]aniline (IVc-I-4)

A solution of 1.29 g (6.1 mmol) of 1-[1-(ethylsulphanyl)ethyl]-3-nitrobenzene is hydrogenated for 12 hours at 30° C. in 20 ml of methanol with 4 bar hydrogen and 250 mg palladium on charcoal. When cold, the reaction mixture is filtered and the mother liquor is concentrated under reduced pressure. This gives 800 mg of the desired product; logP (pH2.3): 1.46;

¹H NMR (400 MHz, DMSO-d) δ=6.93 (t, 1H), 6.57-6.56 (m, 1H), 6.49-6.47 (d, 1H), 6.44-6.41 (m, 1H), 4.82 (s, br., 2H), 3.87-3.82 (q, 1H), 2.36-2.31 (q, 2H), 1.44-1.43 (d, 3H), 1.10 (t, 3H).

The following compounds can be prepared analogously:

1-(3-Aminophenyl)-2-methoxyethanol (IVc-I-5); logP (pH2.3): 0.51;

¹H NMR (400 MHz, DMSO-d) δ=6.93 (m, 1H), 6.58 (t 1H), 6.44 (m, 2H), 4.8 (b), 3.34-3.36 (m, 2H), 3.26 (s, 3H).

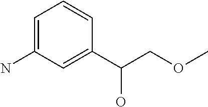

3-[2,2,2-Trifluoro-1-(2-methoxyethoxy)ethyl]aniline (IVc-I-6); logP (pH2.3): 1.64;

¹H NMR (400 MHz, DMSO-d) δ=7.04 (t, 1H), 6.67 (s, 1H), 6.62-6.57 (d, 2H), 5.02 (s, br., 2H), 4.86-4.82 (m, 1H), 3.61-3.58 (m, 2H), 3.49-3.46 (m, 2H), 3.24 (s, 3H).

1-(3-Aminophenyl)-2-methoxyethanol (IVc-I-7); logP (pH2.3): 0.73;

¹H NMR (400 MHz, DMSO-d) δ=6.95 (t, 1H), 6.51 (s, 1H), 6.45-6.41 (d, 2H), 4.97 (s, br., 2H), 4.24-4.19 (m, 1H), 3.29-3.23 (m), 1.27-1.26 (d, 3H), 1.07 (s, 3H).

Preparation of Starting Compounds of the Formula (VIc-I) (cf. Scheme 12)

### 1-[1-(Methylsulphanyl)propyl]-3-nitrobenzene (VIc-I-1)

A solution of 6.5 g (33 mmol) of 1-(1-chloropropyl)-3-nitrobenzene and 3.4 g (49 mmol) of sodium methanethiolate in 7.5 ml of ethanol is stirred for 16 h at 20° C. Thereafter, the reaction mixture is poured into 20 ml of water and extracted with ethyl acetate (3×20 ml). The combined organic phases are dried over MgSO₄ and freed from the solvent under reduced pressure. Thereafter, the crude product is purified by column chromatography over silica gel, with tert-butyl methyl ether/petroleum ether 1:1 as the mobile phase. This gives 5.4 g of the desired product; logP (pH2.3): 3.51;

The following compounds can be prepared analogously:

1-[1-(Methylsulphanyl)ethyl]-3-nitrobenzene (VIc-I-2); logP (pH2.3): 3.06;

¹H NMR (400 MHz, DMSO-d) δ=8.17-8.16 (m, 1H), 8.10-8.07 (m, 1H), 7.82-7.80 (d, 1H), 7.62 (t, 1H), 4.16-4.11 (q, 1H), 1.91 (s, 3H), 1.56-1.54 (d, 3H).

1-[1-(Ethylsulphanyl)propyl]-3-nitrobenzene (VIc-I-3); logP (pH2.3): 3.96;

¹H NMR (400 MHz, DMSO-d) δ=8.16-8.15 (m, 1H), 8.10-8.07 (m, 1H), 7.80-7.78 (d, 1H), 7.62 (t, 1H), 4.00 (t, 1H), 2.35-2.29 (q, 2H), 1.93-1.78 (m, 2H), 1.09 (t, 3H), 0.87 (t, 3H).

1-[1-(Ethylsulphanyl)ethyl]-3-nitrobenzene (VIc-I-4); logP (pH2.3): 3.48;

¹H NMR (400 MHz, DMSO-d) δ=8.19-8.18 (m, 1H), 8.09-8.06 (m, 1H), 7.84-7.82 (d, 1H), 7.62 (t, 1H), 4.27-4.21 (q, 1H), 2.39-2.31 (q, 2H), 1.56-1.55 (d, 3H), 1.11 (t, 3H).

Preparation of Starting Compounds of the Formula (VIc-IV) (cf. Scheme 17)

### 1-(1-Ethoxyethyl)-3-nitrobenzene (VIc-IV-1)

A mixture of 2.0 g (11 mmol) of 1-(1-chloropropyl)-3-nitrobenzene and sodium methoxide, which has been prepared by introducing 0.37 g (16 mmol) of sodium into 60 ml of absolute ethanol, is stirred for 6 h at 78° C. Thereafter, the reaction mixture is freed from solvent under reduced pressure and treated with water and ethyl acetate. The organic phase is separated, dried over sodium sulphate and freed from solvent under reduced pressure. This gives 1.1 g of 1-(1-ethoxyethyl)-3-nitrobenzene;

¹H NMR (400 MHz, DMSO-d) δ=8.15-8.05 (m, 2H), 7.79-7.76 (d, 1H), 7.64 (t, 1H), 4.64-4.59 (q, 1H), 3.48-3.29 (m,2H), 1.40-1.37 (d, 3H), 1.12 (s, 3H).

Preparation of Starting Compounds of the Formula (VIIa) (cf. Scheme 13)

### 1-(1-Chloropropyl)-3-nitrobenzene (VIIa-1)

At 20° C., 37.1 g (287 mmol) of N,N-diisopropylethylamine is added in one portion to a solution of 13.0 g (72 mmol) of 1-(3-nitrophenyl)propan-1-ol in 300 ml of dichloromethane and 0.5 ml of dimethylformamide, and the mixture is stirred for 5 minutes. Thereafter, 24.7 g (215 mmol) of methanesulphonyl chloride are slowly added dropwise. When the addition is complete, the reaction mixture is heated for 6 hours at 40° C. After cooling, the mixture is treated with 200 ml of water. The organic phase is separated off, dried over MgSO₄ and freed from solvent under reduced pressure. Thereafter, the crude product is purified by column chromatography over silica gel, using tert-butyl methyl ether/petroleum ether 1:1 as the mobile phase. This gives 14 g of the desired product; logP (pH2.3): 3.31);

¹H NMR (400 MHz, DMSO-d) δ=8.27-8.26 (m, 1H), 8.18-8.15 (m, 1H), 7.92-7.90 (d, 1H), 7.69 (t, 1H), 5.27 (t, 1H), 2.14-2.06 (m, 2H), 0.96 (t, 3H).

Preparation of Starting Compounds of the Formula (Xa) (cf. Scheme 15)

### 4-Chloro-N-[3-(propylsulphanyl)phenyl]-5-(trifluoromethyl)pyrimidin-2-amine (Xa-1)

At 0° C., 72 ml (9.92 mmol) of a 1 M solution of zinc chloride in ether are added dropwise to a solution of 13.0 g (60.0 mmol) of 2,4-dichloro-5-trifluoropyrimidine in a mixture of 120 ml of dichloroethane and 120 ml of tert-butanol, and the mixture is stirred for 1 hour at the same temperature. Then, 10.0 g (60.0 mmol) of 3-(propylsulphanyl)aniline are added, and 9.2 ml of triethylamine in a mixture of 120 ml of dichloroethane and 120 ml of tert-butanol are subsequently added dropwise. The reaction mixture is stirred for 16 hours at 20° C. Thereafter, the mixture is freed from solvent under reduced pressure and stirred with a mixture of 100 ml of water and 100 ml of ethyl acetate. Finally, the organic phase is separated off, dried over MgSO₄ and freed from solvent under reduced pressure. The crude product is then stirred with 100 ml of petroleum ether. This gives 11.5 g of the desired product; logP (pH2.3): 4.99;

¹H NMR (400 MHz, DMSO-d) δ=10.48 (s, br., 1H), 8.77 (s, 1H), 7.74-7.73 (m, 1H), 7.49-7.47 (d, 1H), 7.28 (t, 1H), 7.05-7.03 (d, 1H), 2.93 (t, 2H), 1.69-1.60 (m, 2H), 0.99 (t, 3H).

The following compounds can be prepared analogously:

4-Chloro-N-[3-(propan-2-ylsulphanyl)phenyl]-5-(trifluoromethyl)pyrimidin-2-amine (Xa-2); logP (pH2.3): 4.93;

¹H NMR (400 MHz, DMSO-d) δ=10.49 (s, br., 1H), 8.77 (s, 111), 7.78 (m, 1H), 7.54-7.52 (d, 1H), 7.29 (t, 1H), 7.10-7.08 (m, 1H), 3.48-3.42 (m, 1H), 1.29-1.28 (d, 6H).

4-Chloro-N-{3-[(3 -methylbutyl)sulphanyl]phenyl}-5-(trifluoromethyl)pyrimidin-2-amine (Xa-3); logP (pH2.3): 5.79;

¹H NMR (400 MHz, DMSO-d) δ=10.49 (s, br., 1H), 8.76 (s, 1H), 7.73-7.72 (m, 1H), 7.50-7.47 (d, 1H), 7.29 (t, 1H), 7.05-7.02 (d, 1H), 2.96 (t, 2H), 1.75-1.67 (m, 1H), 1.55-1.49 (m, 2H), 0.90-0.89 (d, 6H).

4,5 -Dichloro-N-{3-[(3-methylbutyl)sulphanyl]phenyl}pyrimidin-2-amine (Xa-4); logP (pH2.3): 4.98;

¹H NMR (400 MHz, DMSO-d) δ=9.30 (s, br., 1H), 8.35 (s, 1H), 7.65-7.64 (m, 1H), 7.44-7.41 (d, 1H), 7.30 (t, 1H), 7.12-7.10 (d, 1H), 2.97 (t, 2H), 1.76-1.69 (m, 1H), 1.55-1.50 (q, 2H), 0.91-0.89 (d, 6H).

4,5-Dichloro-N-[3-(propylsulphanyl)phenyl]pyrimidin-2-amine (Xa-5); logP (pH2.3): 4.10;

¹H NMR (400 MHz, DMSO-d) δ=9.31 (s, br., 1H), 8.35 (s, 1H), 7.65-7.64 (m, 1H), 7.44-7.41 (d, 1H), 7.30 (t, 1H), 7.12-7.10 (d, 1H), 2.93 (t, 2H), 1.70-1.56 (m, 2H), 0.99 (t, 3H).

4,5-Dichloro-N-[3-(propan-2-ylsulphanyl)phenyl]pyrimidin-2-amine (Xa-6); logP (pH2.3): 4.04;

¹H NMR (400 MHz, DMSO-d) δ=9.34 (s, br., 1H), 8.35 (s, 1H), 7.68 (t, 1H), 7.48-7.46 (m, 1H), 7.32 (t, 1H), 7.17-7.14 (m, 1H), 3.50-3.43 (m, 1H), 1.30-1.28 (d, 6H).

4-Chloro-N-{3-[(2-methylpropyl)sulphanyl]phenyl}-5-(trifluoromethyl)pyrimidin-2-amine (Xa-7); logP (pH2.3): 5.40;

¹H NMR (400 MHz, DMSO-d) δ=10.49 (s, br., 1H), 8.77 (s, br., 1H), 7.73 (s, 1H), 7.49-7.47 (m, 1H), 7.27 (t, 1H), 7.05-7.03 (m, 1H), 2.86-2.84 (d, 2H), 1.89-1.83 (m, 1H), 1.01-1.00 (d, 6H).

4-Chloro-N-{3-[1-(methylsulphanyl)ethyl]phenyl}-5-(trifluoromethyl)pyrimidin-2-amine (Xa-8); logP (pH2.3): 4.50;

¹H NMR (400 MHz, DMSO-d) δ=10.44 (s, br., 1H), 8.74 (s, br., 1H), 7.68-7.67 (s, 1H), 7.57-7.55 (d, 1H), 7.30 (t, 1H), 7.09-7.07 (d, 1H), 3.93-3.88 (q, 1H), 1.93 (s, 3H), 1.53-1.51 (d, 3H).

4-Chloro-N-[3-(1-methoxyethyl)phenyl]-5-(trifluoromethyl)pyrimidin-2-amine (Xa-9); logP (p112.3): 3.89.

4,5-Dichloro-N-[3-(ethylsulphanyl)phenyl]pyrimidin-2-amine (Xa-10); logP (p12.3): 3.63;

¹H NMR (400 MHz, DMSO-d) δ=9.14 (s, br., 1H), 8.35 (s, 1H), 7.64-7.63 (m, 1H), 7.44-7.42 (m, 1H), 7.31 (t, 1H), 7.12-7.09 (d, 1H), 3.01-2.94 (q, 2H), 1.29 (t, 3H).

4-Chloro-N-[3-(ethylsulphanyl)phenyl]-5-(trifluoromethyl)pyrimidin-2-amine (Xa-10); logP (pH2.3): 4.53;

¹H NMR (400 MHz, DMSO-d) δ=10.47 (s, br., 1H), 8.77 (s, 1H), 7.74-7.73 (m, 1H), 7.50-7.47 (m, 1H), 7.28 (t, 1H), 7.05-7.03 (d, 1H), 3.01-2.94 (q, 2H), 1.28 (t, 3H).

4-Chloro-N-[3-(methylsulphanyl)phenyl]-5-(trifluoromethyl)pyrimidin-2-amine (Xa-11); logP (p12.3): 4.11; ¹H NMR (400 MHz, DMSO-d) δ=10.48 (s, br., 1H), 8.77 (s, 1H), 7.70-7.69 (m, 1H), 7.48-7.45 (m, 1H), 7.28 (t, 1H), 7.02-6.99 (m, 1H).

4-Chloro-N-{3-[1-(ethylsulphanyl)ethyl]phenyl}-5-(trifluoromethyl)pyrimidin-2-amine (Xa-12); logP (pH2.3): 4.90;

¹H NMR (400 MHz, DMSO-d) δ=10.44 (s, br., 1H), 8.74 (s, br., 1H), 7.70-7.68 (m, 1H), 7.57-7.54 (d, 1H), 7.29 (t, 1H), 7.10-7.08 (d, 1H), 4.04-3.99 (q, 1H), 2.41-2.35 (q, 2H), 1.52-1.50 (d, 3H), 1.12 (s, 3H).

5-Bromo-4-chloro-N-[3-(1-methoxyethyl)phenyl]pyrimidin-2-amine (Xa-13); logP (pH2.3): 3.09;

¹H NMR (400 MHz, DMSO-d) δ=9.12 (s, br., 1H), 8.42 (s, 1H), 7.51-7.48 (m, 2H), 7.35 (t, 1H), 7.12-7.10 (d, 1H), 4.34-4.30 (m, 1H), 3.16 (s), 1.39-1.33 (m, 3H).

1-{3-[(4,5-Dichloropyrimidin-2-yl)amino]phenyl}ethanol (Xa-14); logP (pH2.3): 1.98;

¹H NMR (400 MHz, DMSO-d) δ=9.32 (s, br., 1H), 8.32 (s, 1H), 7.54 (s, br., 1H), 7.47-7.45 (d, 1H), 7.31 (t, 1H), 7.18-7.16 (d, 1H), 5.01 (s, br., 1H), 4.75-4.72 (m, br., 1H), 1.36-1.35 (d, 3H).

1-(3-{[4-Chloro-5-(trifluoromethyl)pyrimidin-2-yl]amino}phenyl)ethanol (Xa-15); logP (pH2.3): 2.81;

¹H NMR (400 MHz, DMSO-d) δ=10.41 (s, br., 1H), 8.74 (s, br., 1H), 7.64 (s, 1H), 7.56-7.53 (d, 1H), 7.28 (t, 1H), 7.10-7.08 (d, 1H), 4.73-4.68 (q, 1H), 1.35-1.33 (d, 3H).

Preparation of Starting Compounds of the Formula (Xb) and (Xc)

### 4-Chloro-N-[3-(propylsulphinyl)phenyl]-5-(trifluoromethyl)pyrimidin-2-amine (Xb-1) and 4-chloro-N-[3-(propylsulphonyl)phenyl]-5-(trifluoromethyl)pyrimidin-2-amine (Xc-1)

At 20° C., 2.45 g (9.92 mmol) of 70% strength 3-chlorobenzenecarboperoxo acid are added portionwise to a solution of 2.30 g (6.61 mmol) of 4-chloro-N-[3-(propylsulphanyl)phenyl]-5(trifluoromethyl)pyrimidin-2-amine in 20 ml of dichloromethane, and the mixture is stirred for 16 hours at the same temperature. After 16 hours, the reaction mixture is stirred with 20 ml of 2 M sodium hydroxide solution and, if tested positive for peroxide, with 10 ml of aqueous sodium bisulphite solution. Thereafter, the organic phase is separated off, washed with 20 ml of water, dried over MgSO₄ and freed from solvent under reduced pressure. The crude product is then subjected to fractional chromatography over silica gel, with tert-butyl methyl ether as the mobile phase. This gives 1.1 g of 4-chloro-N-[3-(propylsulphinyl)phenyl]-5-(trifluoromethyl)pyrimidin-2-amine; logP (pH2.94): 1.76;

¹H NMR (400 MHz, DMSO-d) δ=10.70 (s, br., 1H), 8.80 (s, 1H), 8.03-8.02 (m, 1H), 7.81-7.79 (d, 1H), 7.54 (t, 1H), 7.36-7.34 (d, 1H), 2.92-2.73 (m, 2H), 1.73-1.49 (m, 2H), 0.98 (t, 3H)

and 1.0 g of 4-chloro-N-[3-(propylsulphonyl)phenyl]-5-(trifluoromethyl)pyrimidin-2-amine; logP (pH2.3): 3.29;

¹H NMR (400 MHz, DMSO-d) δ=11.00 (s, br., 1H), 8.89 (s, 1H), 8.32 (s, br., 1H), 8.00-7.98 (d, 1H), 7.66 (t, 1H), 7.61-7.58 (m, 1H), 3.31-3.34 (m, 2H), 1.62-1.56 (m, 2H), 0.93 (t, 3H).

Preparation of Starting Compounds of the Formula (Ic-IV) (cf. Scheme 4)

### 1-(3-{[5-Chloro-4-(cyclopropylamino)pyrimidin-2-yl]amino}phenyl)ethanol (Ic-IV-1)

A mixture of 10.0 g (49 mmol) of 2,5-dichloro-N-cyclopropylpyrimidin-4-amine, 8.4 g (61 mmol) of 1-(3-aminophenyl)ethanol and 1.7 g (10 mmol) of 4-toluenesulphonic acid in 40 ml of 1,4-dioxane is stirred for 16 hours at 100° C. The organic phase is subsequently freed from solvent under reduced pressure. The residue is then stirred with a mixture of ethyl acetate and sodium hydrogen carbonate solution. The organic is separated off, washed with water, dried over MgSO₄ and freed from solvent under reduced pressure. This gives 10.0 g of 1-(3-{[5-chloro-4-(cyclopropylamino)pyrimidin-2 -yl]amino}phenyl)ethanol; logP (pH2.94): 1.25;

¹H NMR (400 MHz, DMSO-d) δ=8.94 (s, br., 1H), 7.89 (s, 1H), 7.86 (s, br., 1H), 7.64-7.61 (d, 1H), 7.15 (t, 1H), 6.94 (s, br., 1H), 6.89-6.88 (d, 1H), 4.83-4.82 (d, 1H), 4.69-4.52 (m, 1H), 2.95-2.88 (m, 1H), 1.32-1.30 (d, 3H), 0.81-0.78 (m, 2H), 0.67-0.63 (m, 2H).

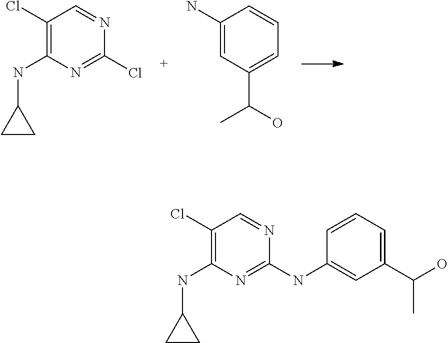

The following compounds can be prepared analogously:

1-(3 -{[5-Bromo-4-(cyclopropylamino)pyrimidin-2-yl]amino}phenyl)ethanol (Ic-IV-2); logP (pH2.3): 1.31;

¹H NMR (400 MHz, DMSO-d) δ=9.20 (s, br., 1H), 8.00 (s, 1H), 7.91 (s, br., 1H), 7.66-7.63 (d, 1H), 7.16 (t, 1H), 6.96 (s, br., 1H), 6.89-6.87 (d, 1H), 5.04-5.03 (d, 1H), 4.67-4.61 (m, 1H), 2.92-2.85 (m, 1H), 1.30-1.25 (d, 3H), 0.83-0.76 (m, 2H), 0.68-0.64 (m, 2H).

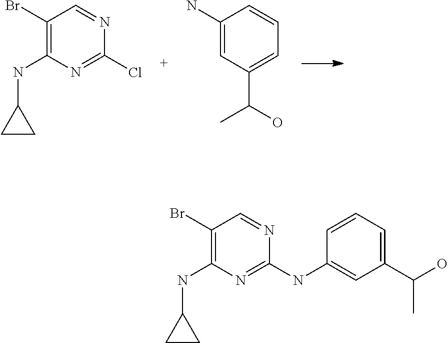

1-(3 -{[4-(Cyclopropylamino)-5-(trifluoromethyl)pyrimidin-2-yl]amino}phenyl)ethanol (Ic-IV-3); logP (pH2.3): 2.01;

¹H NMR (400 MHz, DMSO-d) δ=9.60 (s, br., 1H), 8.18 (s, 1H), 7.94 (s, br., 1H), 7.70-7.68 (d, 1H), 7.20 (t, 1H), 7.06 (s, br., 1H), 6.94-6.90 (d, 1H), 5.06-5.05 (d, 1H), 4.68-4.63 (m, 1H), 2.97-2.90 (m, 1H), 1.30-1.25 (d, 3H), 0.84-0.79 (m, 2H), 0.69-0.66 (m, 2H).

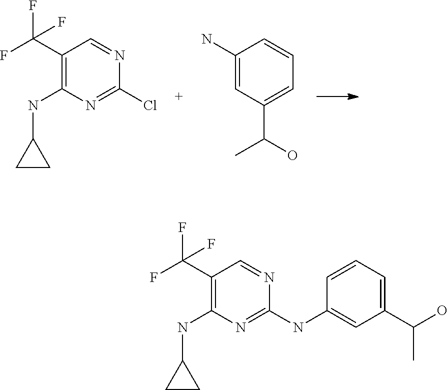

Preparation of Starting Compounds of the Formula (VIc-III) (cf. Scheme 18)

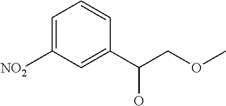

### 2-Methoxy-1-(3-nitrophenyl)ethanol

590 mg (10.9 mmol) of sodium methoxide are introduced into 50 ml of methanol, the mixture is treated with 100 mg of 2-(3-nitrophenyl)oxirane (0.6 mmol) and stirred for 50 h at room temperature. The mixture is subsequently evaporated completely on a rotary evaporator, and the residue is treated with 30 ml of 1 N perchloric acid (to pH 7-8). After five extractions with in each case 20 ml of ethyl acetate and subsequent removal of the solvent on a rotary evaporator, 80 mg (83%) of 2-methoxy-1-(3-nitrophenyl)ethanol are obtained (logP (pH2.3): 1.35); ¹H NMR (400 MHz, MeOH-d) δ=8.1-8.3 (m, 2 H), 7.55-7.8 (m, 2H), 4.93 (m 1H), 3.54 (d, 2H), 3.70 (s, 3H).

### EXAMPLES

The compounds of the formula I, (Ia), (Ib), (Ic), which are listed in Table 1 hereinbelow, can be obtained analogously to the methods detailed above.

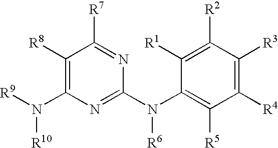

| Ex | R¹ | R² | R³ | R⁴ | R⁵ | R⁶ | R⁷ | R⁸ | R⁹ | R¹⁰ | log p |
|---|---|---|---|---|---|---|---|---|---|---|---|
| 1 | H | H | (1-methoxypropan- | H | H | H | H | Br | H | cyclopropyl | 1.83[b] |
| | | | 2-yl)oxy | | | | | | | | |
| 2 | H | H | 2-(2- | H | H | H | H | Br | H | cyclopropy | 1.69[b] |
| | | | methoxyethoxy)- | | | | | | | | |
| | | | ethoxy | | | | | | | | |
| 3 | H | (2-methylprop-2-en-1-yl)- | H | H | H | H | H | Br | H | cyclopropyl | 4.25[b] |
| | | sulphanyl | | | | | | | | | |
| 4 | H | chlorine | (1-methoxypropan- | H | H | H | H | Br | H | cyclopropyl | 2.75[b] |
| | | | 2-yl)oxy | | | | | | | | |
| 5 | H | propylsulphanyl | H | H | H | H | H | Cl | H | cyclobutyl | 3.66[b] |
| 6 | H | butan-2-ylsulphanyl | H | H | H | H | H | Cl | H | cyclobutyl | 4.14[b] |
| 7 | H | methoxymethyl | H | H | H | H | H | Cl | H | cyclopentyl | 2.23[b] |
| 8 | H | methoxymethyl | H | H | H | H | H | F | H | cyclobutyl | 1.5[a]; |
| | | | | | | | | | | | 2.95[c] |
| 9 | H | methoxymethyl | H | H | H | H | H | Cl | H | 2-methyl-3-oxobutan- | 1.99[a] |
| | | | | | | | | | | 2-yl | |
| 10 | H | 2,2,2-trifluoro- | H | H | H | H | H | Br | H | cyclobutyl | 3[b] |
| | | 1-methoxyethyl | | | | | | | | | |
| 11 | H | 1-(ethylsulphanyl)ethyl) | H | H | H | H | H | Cl | H | cyclobutyl | 3.25[b] |
| 12 | H | 1-(ethylsulphanyl)ethyl | H | H | H | H | H | Br | H | cyclobutyl | 3.49[b] |
| 13 | H | 1-(methylsulphanyl)ethyl | H | H | H | H | H | Br | H | cyclobutyl | 3.07[b] |
| 14 | H | 1-(methylsulphanyl)ethyl | H | H | H | H | H | Cl | H | cyclobutyl | 2.82[b] |
| 15 | H | 1-methoxyethyl | H | H | H | H | H | Cl | H | cyclobutyl | 2.27[b] |
| 16 | H | 1-(methylsulphanyl)ethyl | H | H | H | H | H | CF₃ | H | cyclopentyl | 4.48[b] |
| 17 | H | 1-methoxyethyl | H | H | H | H | H | CF₃ | H | cyclopentyl | 4.13[b] |
| 18 | H | 1-(methylsulphanyl)ethyl | H | H | H | H | H | CF₃ | H | cyclobutyl | 4.34[b] |
| 19 | H | 1-methoxyethyl | H | H | H | H | H | CF₃ | H | cyclobutyl | 3.69[b] |
| 20 | H | 1-methoxyethyl | H | H | H | H | H | Br | H | cyclobutyl | 2.46[b] |
| 21 | H | 2,2,2-trifluoro- | H | H | H | H | H | Cl | H | cyclobutyl | 2.99[b] |
| | | 1-methoxyethyl | | | | | | | | | |
| 22 | H | methoxymethyl | H | H | H | H | H | Cl | H | 2,2-difluoroethyl | 1.99[b] |
| 23 | H | 2,2,2-trifluoro- | H | H | H | H | H | Cl | H | 2,2-difluoroethyl | 2.93[b] |
| | | 1-methoxyethyl | | | | | | | | | |
| 24 | H | methoxymethyl | H | H | H | H | H | CF₃ | H | cyclobutyl | 3.3[b] |
| 25 | H | 2,2,2-trifluoro- | H | H | H | H | H | CF₃ | H | cyclobutyl | 4.18[b] |
| 26 | H | methoxymethyl | H | H | H | H | H | Cl | H | propan-2-yl | 1.76[b] |
| 27 | H | ethylsulphonyl | H | H | H | H | H | Cl | H | cyclobutyl | 2.21[b] |
| 28 | H | ethylsophinyl | H | H | H | H | H | Cl | H | cyclobutyl | 1.76[b] |
| 29 | H | methoxymethyl | H | H | H | H | H | Cl | CH₃ | propan-2-yl | 2.56[a]; |
| | | | | | | | | | | | 4[c] |
| 30 | H | propylsulphanyl | H | H | H | H | H | Cl | H | 2,2,2-trifluoroethyl | 4.08[b] |
| 31 | H | propylsulphanyl | H | H | H | H | H | Cl | H | propan-2-yl | 3.3[b] |
| 32 | H | (ethylsulphanyl)methyl | H | H | H | H | H | Cl | H | 2,2,2-trifluoroethyl | 3.49[b] |
| 33 | H | (ethylsulphanyl)methyl | H | H | H | H | H | Cl | H | propan-2-yl | 2.61[b] |
| 34 | H | propylsulphonyl | H | H | H | H | H | CF₃ | H | cyclobutyl | 3.46[b] |
| 35 | H | propylsulphinyl | H | H | H | H | H | CF₃ | H | cyclobutyl | 2.97[b] |
| 36 | H | 1-(ethylsulphanyl)ethyl) | H | H | H | H | H | CF₃ | H | cyclobutyl | 4.75[b] |
| 37 | H | 1-(ethylsulphanyl)ethyl | H | H | H | H | H | CF₃ | H | cyclopentyl | 5.2[b] |
| 38 | H | (2-methylprop-2-en-1-yl)- | H | H | H | H | H | Cl | H | cyclopropyl | 3.59[b] |
| | | sulphanyl | | | | | | | | | |
| 39 | H | 1-(ethylsulphonyl)ethyl) | H | H | H | H | H | CF₃ | H | cyclopentyl | 3.27[b] |
| 40 | H | 1-(ethylsulphonyl)ethyl) | H | H | H | H | H | CF₃ | H | cyclobutyl | 2.89[b] |
| 41 | H | 1-(ethylsulphonyl)ethyl) | H | H | H | H | H | CF₃ | H | cyclopropylmethyl | 2.65[b] |
| 42 | H | 1-(ethylsulphinyl)ethyl) | H | H | H | H | H | CF₃ | H | cyclopentyl | 2.87[b] |
| 43 | H | 1-(ethylsulphinyl)ethyl) | H | H | H | H | H | CF₃ | H | cyclobutyl | 2.55[b] |
| 44 | H | 1-(ethylsulphinyl)ethyl) | H | H | H | H | H | CF₃ | H | cyclopropylmethyl | 2.3[b] |
| 45 | H | 1-(ethylsulphinyl)ethyl) | H | H | H | H | H | CF₃ | H | cyclopropyl | 2.2[b] |
| 46 | H | 1-(ethylsulphonyl)ethyl) | H | H | H | H | H | CF₃ | H | cyclopropyl | 2.49[b] |
| 47 | H | methoxymethyl | H | Methoxy- | H | H | H | Cl | H | cyclobutyl | 2.06[b] |
| | | | | methyl | | | | | | | |
| 48 | H | 2-ethoxyethoxy | CH₃ | H | H | H | H | Cl | H | cyclopropyl | 2.23[b] |
| 49 | H | (2-chloroethyl)sulphonyl | chloro | H | H | H | H | Cl | H | cyclopropyl | 2.67[b] |
| 50 | H | phenoxymethyl | H | H | H | H | H | Cl | H | cyclopropyl | 2.92[b] |
| 51 | H | methoxymethyl | H | H | H | H | H | Cl | H | cyanomethyl | 1.68[a]; |
| | | | | | | | | | | | 2.09[c] |
| 52 | H | methoxymethyl | H | H | H | H | H | Cl | H | 3-(2-oxoazepan-1-yl)- | 1.67[a] |
| | | | | | | | | | | propyl | |
| 53 | H | 2-methoxymethyl | H | H | H | H | H | Cl | H | cyclopropyl | 1.78[b] |
| 54 | H | H | ethylsulphonyl | H | H | H | H | Cl | H | cyclopropyl | 2.04[b] |
| 55 | H | H | ethylsuplhonyl | H | H | H | H | CF₃ | H | cyclopropyl | 2.66[b] |
| 56 | H | 2-methoxymethyl | H | H | H | H | H | Br | H | cyclobutyl | 2.23[b] |
| 57 | H | propylsulphanyl | H | H | H | H | H | Br | H | cyclobutyl | 3.96[b] |
| 58 | H | methoxymethyl | H | H | H | H | H | Cl | CH₃ | cyclopropyl | 3.76[c]; |
| | | | | | | | | | | | 2.7[b] |
| 59 | H | 2-methoxypropan-2-yl | H | H | H | H | H | Cl | H | cyclopropyl | 3.5[c]; |
| | | | | | | | | | | | 2.16[b] |
| 60 | H | 2-methoxyethyl | H | H | H | H | H | Cl | H | cyclopropyl | 2.41[b] |
| 61 | H | 1-(methylsulphanyl)propyl | H | H | H | H | H | Cl | H | cyclopropyl | 2.94[b] |
| 62 | H | 1-(methylsulphanyl)propyl | H | H | H | H | H | CF₃ | H | cyclopropyl | 4.23[b] |
| 63 | H | propylsulphanyl | H | H | H | H | H | Cl | H | 2-methylcyclopropyl | 3.58[b] |
| 64 | H | 2-methoxy-2-methylpropyl | H | H | H | H | H | Cl | H | cyclopropyl | 3.81[c]; |
| | | | | | | | | | | | 2.21[b] |
| 65 | H | methoxymethyl | H | H | H | H | H | Cl | H | 2-methylcyclopropyl | 3.3[c]; |
| | | | | | | | | | | | 2.03[b] |
| 66 | H | 2-methoxyethoxy | H | H | H | H | H | Cl | H | cyclobutyl | 2.12[b] |
| 67 | H | 2-methoxyethoxy | H | H | H | H | H | Br | H | cyclopropyl | 1.95[b] |
| 68 | H | chloro | 2-methoxyethoxy | H | H | H | H | Cl | H | cyclopropyl | 2.12[b] |
| 69 | H | methoxymethyl | H | H | H | H | H | Cl | CH₃ | 3-(2-oxoazepan-1- | 1.91[b] |
| | | | | | | | | | | yl)proply | |
| 70 | H | methoxymethyl | H | H | H | H | H | Br | H | propan-2-yl | 1.92[b] |
| 71 | H | 2-methoxyethoxy | H | H | H | H | H | CF₃ | H | cyclobutyl | 3.33[b] |
| 72 | H | CF₃ | (1-methoxypropan- | H | H | H | H | Cl | H | cyclopropyl | 2.91[b] |
| | | | 2-yl)oxy | | | | | | | | |
| 73 | H | H | 2-methoxyethoxy | H | H | H | H | Cl | H | cyclopropyl | 1.47[b] |
| 74 | H | 1-(ethylsulphinyl)ethyl | H | H | H | H | H | Cl | H | cyclopropyl | 2.16[c]; |
| | | | | | | | | | | | 1.48[b] |
| 75 | H | 1-(ethylsulphonyl)ethyl | H | H | H | H | H | Cl | H | cyclopropyl | 2.54[b]; |
| | | | | | | | | | | | 1.62[b] |
| 76 | H | 1-(ethylsulphinyl)ethyl | H | H | H | H | H | Cl | H | cyclobutyl | 2.53[c]; |
| | | | | | | | | | | | 1.68[b] |
| 77 | H | 1-(ethylsulphonyl)ethyl | H | H | H | H | H | Cl | H | cyclobutyl | 2.98[c]; |
| | | | | | | | | | | | 1.9[b] |
| 78 | H | chloro | (1-methoxypropan- | H | H | H | H | CF₃ | H | cyclopropyl | 3.99[c]; |
| | | | 2-yl)oxy | | | | | | | | 3.7[b] |
| 79 | H | 2-ethoxyethoxy | CH₃ | H | H | H | H | CF₃ | H | cyclopropyl | 4.15[c]; |
| | | | | | | | | | | | 3.66[b] |
| 80 | H | CF₃ | (1-methoxypropan- | H | H | H | H | CF₃ | H | cyclopropyl | 4.28[c]; |
| | | | 2-yl)oxy | | | | | | | | 4.05[b] |
| 81 | H | fluorine | 2-methoxyethoxy | H | H | H | H | CF₃ | H | cyclopropyl | 3.27[c]; |
| | | | | | | | | | | | 2.89[b] |
| 82 | H | H | 2-methoxyethoxy | H | H | H | H | CF₃ | H | cyclopropyl | 3.06[c]; |
| | | | | | | | | | | | 2.3[b] |
| 83 | H | 1-methoxyethyl | H | H | H | H | H | Cl | H | 2,2,2-trifluoroethyl | 3.18[c]; |
| | | | | | | | | | | | 2.82[b] |
| 84 | H | methoxymethyl | H | H | H | H | H | Br | H | 2-methylcyclopropyl | 3.59[c]; |
| | | | | | | | | | | | 2.15[b] |
| 85 | H | methoxymethyl | H | H | H | H | H | Cl | CH₃ | ethyl | 3.7[c]; |
| | | | | | | | | | | | 2.14[b] |
| 86 | H | fluorine | H | H | H | H | H | Cl | H | cyclopropyl | 1.87[b] |
| 87 | H | methoxymethyl | H | H | H | H | H | Cl | butylene | | 3.83[c]; |
| | | | | | | | | | | | 1.95[b] |
| 88 | H | H | (1-methoxypropan- | H | H | H | H | CF₃ | H | cyclopropyl | 3.45[c]; |
| | | | 2-yl)oxy | | | | | | | | 2.71[b] |
| 89 | H | methoxymethyl | H | H | H | H | H | Cl | pentane-1,5-diyl | | 4.2[c]; |
| | | | | | | | | | | | 3.14[b] |
| 90 | H | methoxymethyl | H | H | H | H | H | Cl | —CH₂CH₂OCH₂CH₂— | | 2.87[c]; |
| | | | | | | | | | | | 2.4[b] |
| 91 | H | H | 2-ethoxyethoxy | H | H | H | H | CF₃ | H | cyclopropyl | 3.64[c]; |
| | | | | | | | | | | | 3.26[b] |
| 92 | H | 1-methoxyethyl | CH₃ | H | H | H | H | Cl | H | cyclopropyl | 3.7[c]; |
| | | | | | | | | | | | 2.11[b] |
| 93 | H | 2-ethoxyethoxy | H | H | H | H | H | Cl | H | cyclopropyl | 3.3[c]; |
| | | | | | | | | | | | 2.13[b] |
| 94 | H | methoxymethyl | H | H | H | H | H | Cl | —CH₂CH₂OCH₂CH₂— | | 3.76[c]; |
| | | | | | | | | | | | 3.25[b] |
| 95 | H | methoxymethyl | H | H | H | H | H | Cl | H | ethyl | 2.84[c]; |
| | | | | | | | | | | | 1.47[b] |
| 96 | H | H | propylsulphanyl | H | H | H | H | Cl | H | cyclopropyl | 3.09[b] |
| 97 | H | H | propylsulphonyl | H | H | H | H | Cl | H | cyclopropyl | 2.76[c]; |
| | | | | | | | | | | | 2.42[b] |
| 98 | H | H | propylsulphinyl | H | H | H | H | Cl | H | cyclopropyl | 1.78[b] |
| 99 | H | propylsulphanyl | H | H | H | H | H | Br | H | 2-methylcyclopropyl | 5.09[c]; |
| | | | | | | | | | | | 3.83[b] |
| 100 | H | propylsulphinyl | H | H | H | H | H | Cl | H | cyclobutyl | 2.09[b] |
| 101 | H | ethylsulphanyl | H | H | H | H | H | CF₃ | H | cyclopropyl | 4.21[c]; |
| | | | | | | | | | | | 3.95[b] |
| 102 | H | propylsulphinyl | H | H | H | H | H | Br | H | cyclopropyl | 2.5[c]; |
| | | | | | | | | | | | 1.04[b] |
| 103 | H | propylsulphonyl | H | H | H | H | H | Cl | H | cyclobutyl | 3.27[c]; |
| | | | | | | | | | | | 2.6[b] |
| 104 | H | butylsulphanyl | H | H | H | H | H | Br | H | cyclobutyl | 5.71[c]; |
| | | | | | | | | | | | 4.57[b] |
| 105 | H | butylsulphanyl | H | H | H | H | H | Br | H | cyclopropyl | 4.99[c]; |
| | | | | | | | | | | | 4[b] |
| 106 | H | butylsulphanyl | H | H | H | H | H | Cl | H | cyclobutyl | 5.13[c]; |
| | | | | | | | | | | | 4.28[b] |
| 107 | H | butylsulphanyl | H | H | H | H | H | CF₃ | H | cyclopropyl | 4.94[b] |
| 108 | H | methoxymethyl | H | H | H | H | H | Cl | H | 2-ethylcyclopropyl | 2.46[b] |
| 109 | H | propylsulphanyl | H | H | H | H | H | CF₃ | H | cyclopropyl | 4.44[b] |
| 110 | H | propylsulphonyl | H | H | H | H | H | Br | H | cyclopropyl | 2.99[c]; |
| | | | | | | | | | | | 2.43[b] |
| 111 | H | ethylsulphonyl | H | H | H | H | H | Br | H | cyclobutyl | 3.06[c]; |
| | | | | | | | | | | | 2.36[b] |
| 112 | H | ethylsulphinyl | H | H | H | H | H | Br | H | cyclobutyl | 2.63[c]; |
| | | | | | | | | | | | 1.88[b] |
| 113 | H | ethylsulphonyl | H | H | H | H | H | Cl | H | cyclopropyl | 2.48[c]; |
| | | | | | | | | | | | 1.87[b] |
| 114 | H | ethylsulphinyl | H | H | H | H | H | Cl | H | cyclopropyl | 1.49[a]; |
| | | | | | | | | | | | 2.11[c]; |
| | | | | | | | | | | | 1.48[b] |
| 115 | H | 1-(ethylsulphanyl)propyl | H | H | H | H | H | Cl | H | cyclopropyl | 4.69[c]; |
| | | | | | | | | | | | 3.33[b] |
| 116 | H | ethylsulphonyl | H | H | H | H | H | CF₃ | H | cyclopropyl | 2.8[c]; |
| | | | | | | | | | | | 2.74[b] |
| 117 | H | ethylsulphinyl | H | H | H | H | H | CF₃ | H | cyclopropyl | 2.46[c]; |
| | | | | | | | | | | | 2.2[b] |
| 118 | H | ethylsulphonyl | H | H | H | H | H | CF₃ | H | cyclobutyl | 3.23[c]; |
| | | | | | | | | | | | 3.07[b] |
| 119 | H | ethylsulphinyl | H | H | H | H | H | CF₃ | H | cyclobutyl | 2.81[c]; |
| | | | | | | | | | | | 2.61[b] |
| 120 | H | 1-(ethylsulphonyl)propyl | H | H | H | H | H | Cl | H | cyclopropyl | 2.83[c]; |
| | | | | | | | | | | | 1.94[b] |
| 121 | H | butylsulphonyl | H | H | H | H | H | CF₃ | H | cyclopropyl | 3.46[c]; |
| | | | | | | | | | | | 3.4[b] |
| 122 | H | butylsulphinyl | H | H | H | H | H | CF₃ | H | cyclopropyl | 3.02[c]; |
| | | | | | | | | | | | 2.94[b] |
| 123 | H | butylsulphonyl | H | H | H | H | H | Cl | H | cyclopropyl | 3.18[c]; |
| | | | | | | | | | | | 2.65[b] |
| 124 | H | butylsulphinyl | H | H | H | H | H | Cl | H | cyclopropyl | 2.71[c]; |
| | | | | | | | | | | | 2.13[b] |
| 125 | H | butylsulphonyl | H | H | H | H | H | Br | H | cyclopropyl | 3.32[c]; |
| | | | | | | | | | | | 2.81[b] |
| 126 | H | butylsulphinyl | H | H | H | H | H | Br | H | cyclopropyl | 2.86[c]; |
| | | | | | | | | | | | 2.28[b] |
| 127 | H | methoxymethyl | H | H | H | H | H | CF₃ | H | cyclopropylmethyl | |
| 128 | H | butylsulphanyl | H | H | H | H | H | CF₃ | H | cyclobutyl | 5.78[c]; |
| | | | | | | | | | | | 5.55[b] |
| 129 | H | 1-methoxyethyl | H | H | H | H | H | Cl | H | propan-2-yl | 3.67[c]; |
| | | | | | | | | | | | 2.02[b] |
| 130 | H | 1-methoxyethyl | H | H | H | H | H | Cl | H | 1-(1-chloro- | 4.65[c]; |
| | | | | | | | | | | cyclopropyl)ethyl | 3.72[b] |
| 131 | H | 1-(ethylsulphanyl)ethyl | H | H | H | H | H | Cl | H | propan-2-yl | 4.65[c]; |
| | | | | | | | | | | | 2.91[b] |
| 132 | H | 1-(butan-2-ylsulphanyl)ethyl | H | H | H | H | H | CF₃ | H | cyclopropyl | 5.38[c]; |
| | | | | | | | | | | | 5.09[b] |
| 133 | H | 1-(butan-2-ylsulphanyl)ethyl | H | H | H | H | H | Cl | H | cyclopropyl | 5.08[c]; |
| | | | | | | | | | | | 3.79[b] |
| 134 | H | 1-(pentan-2- | H | H | H | H | H | Cl | H | cyclopropyl | 5.55[c]; |
| | | ylsulphanyl)ethyl | | | | | | | | | 4.24[b] |
| 135 | H | 1-(pentan-2- | H | H | H | H | H | CF₃ | H | cyclopropyl | 5.8[c]; |
| | | ylsulphanyl)ethyl | | | | | | | | | 5.53[b] |
| 136 | H | methoxymethyl | H | H | H | H | H | Cl | cyclo- | propyl | 3.68[b] |
| | | | | | | | | | propyl | | |
| 137 | H | methoxymethyl | H | H | H | H | H | Cl | cyclo- | ethyl | 4.23[c]; |
| | | | | | | | | | propyl | | 3.24[b] |
| 138 | H | methoxymethyl | H | H | H | H | H | Cl | H | 2-fluoroethyl | 2.57[c]; |
| | | | | | | | | | | | 1.48[b] |
| 139 | H | methoxymethyl | H | H | H | H | H | Cl | hexane- | hexane-1,6-diyl | 2.88[b] |
| | | | | | | | | | 1,6-diyl | | |
| 140 | H | methoxymethyl | H | H | H | H | H | Cl | ethyl | propan-2-yl | 3[b] |
| 141 | H | methoxymethyl | H | H | H | H | H | Cl | CH₃ | propyl | 2.55[b] |
| 142 | H | methoxymethyl | H | H | H | H | H | Cl | H | 2,2-dimethylpropyl | 2.48[b] |
| 143 | H | methoxymethyl | H | H | H | H | H | Cl | H | pentan-2-yl | 2.41[b] |
| 144 | H | methoxymethyl | H | H | H | H | H | Cl | H | pentan-3-yl | 2.3[b] |
| 145 | H | methoxymethyl | H | H | H | H | H | Cl | H | 2-methylbutyl | 2.37[b] |
| 146 | H | methoxymethyl | H | H | H | H | H | Cl | H | 3-methylbutyl | 2.37[b] |
| 147 | H | methoxymethyl | H | H | H | H | H | Cl | H | pentyl | 2.41[b] |
| 148 | H | methoxymethyl | H | H | H | H | H | Cl | H | 2-methylpropyl | 2.08[b] |
| 149 | H | methoxymethyl | H | H | H | H | H | Cl | H | propyl | 1.72[b] |
| 150 | H | 1-(ethylsulphonyl)ethyl | H | H | H | H | H | Cl | H | propan-2-yl | 2.89[c]; |
| | | | | | | | | | | | 1.65[b] |
| 151 | H | butylsulphonyl | H | H | H | H | H | Br | H | cyclobutyl | 3.88[c]; |
| | | | | | | | | | | | 3.24[b] |
| 152 | H | butylsulphinyl | H | H | H | H | H | Cl | H | cyclobutyl | 3.5[c]; |
| | | | | | | | | | | | 2.67[b] |
| 153 | H | 1-(ethylsulphonyl)ethyl | H | H | H | H | H | Cl | H | cyclobutyl | 2.03[b] |
| 154 | H | 1-(ethylsulphonyl)ethyl | H | H | H | H | H | Cl | H | cyclopropyl | 2.7[c]; |
| | | | | | | | | | | | 1.76[b] |
| 155 | H | propylsulphanyl | H | H | H | H | H | CF₃ | H | cyclobutyl | 5.43[c]; |
| | | | | | | | | | | | 5.01[b] |
| 156 | H | Cl | 2-(2- | H | H | H | H | CF₃ | H | cyclopropyl | 3.2[b] |
| | | | methoxyethoxy)- | | | | | | | | |
| | | | ethoxy | | | | | | | | |
| 157 | H | CH₃ | 2-methoxyethoxy | H | H | H | H | CF₃ | H | cyclopropyl | 2.14[b] |
| 158 | H | Cl | (1-methoxypropan- | H | H | H | H | Cl | H | cyclopropyl | |
| | | | 2-yl)oxy | | | | | | | | |
| 159 | H | propylsulphonyl | H | H | H | H | H | Br | H | cyclobutyl | 2.81[b] |
| 160 | H | 1-(butan-2-ylsulphinyl)ethyl | H | H | H | H | H | CF₃ | H | cyclopropyl | 3.11[c]; |
| | | | | | | | | | | | 2.85[b] |
| 161 | H | (2-methylpropyl)sulphonyl | H | H | H | H | H | Cl | H | cyclopropyl | 3.23[c]; |
| | | | | | | | | | | | 2.64[b] |
| 162 | H | (2-methylpropyl)sulphinyl | H | H | H | H | H | Cl | H | cyclopropyl | 2.81[c]; |
| | | | | | | | | | | | 2.11[b] |
| 163 | H | 1-(butan-2-ylsulphonyl)ethyl | H | H | H | H | H | Cl | H | cyclopropyl | 3.16[c]; |
| | | | | | | | | | | | 2.16[b] |
| 164 | H | 1-(butan-2-ylsulphinyl)ethyl | H | H | H | H | H | Cl | H | cyclopropyl | 2.79[c]; |
| | | | | | | | | | | | 2[b] |
| 165 | H | 1-(pentan-2- | H | H | H | H | H | Cl | H | cyclopropyl | 3.56[c]; |
| | | ylsulphonyl)ethyl | | | | | | | | | 2.48[b] |
| 166 | H | 1-(pentan-2- | H | H | H | H | H | Cl | H | cyclopropyl | 3.1[c]; |
| | | ylsulphinyl)ethyl | | | | | | | | | 2.2[b] |
| 167 | H | 1-(pentan-2- | H | H | H | H | H | CF₃ | H | cyclopropyl | 3.85[c]; |
| | | ylsulphinyl)ethyl | | | | | | | | | 3.34[b] |
| 168 | H | 1-(pentan-2- | H | H | H | H | H | CF₃ | H | cyclopropyl | 3.47[b] |
| | | ylsulphonyl)ethyl | | | | | | | | | |
| 169 | H | 1,2-dimethoxyethyl | H | H | H | H | H | Cl | H | cyclopropyl | |
| 170 | H | butylsulphonyl | H | H | H | H | H | Cl | H | cyclobutyl | 3.69[c]; |
| | | | | | | | | | | | 3.09[b] |
| 171 | H | butylsulphinyl | H | H | H | H | H | Cl | H | cyclobutyl | 3.31[c]; |
| | | | | | | | | | | | 2.48[b] |
| 172 | H | methoxymethyl | H | H | H | H | H | Cl | H | CH₃ | 2.44[c]; |
| | | | | | | | | | | | 1.18[b] |
| 173 | H | Cl | 2-(2- | H | H | H | H | Cl | H | cyclopropyl | 2.12[b] |
| | | | methoxyethoxy)- | | | | | | | | |
| | | | ethoxy | | | | | | | | |
| 174 | H | CH₃ | 2-methoxyethoxy | H | H | H | H | Cl | H | cyclopropyl | 1.43[b] |
| 175 | H | butan-2-ylsulphinyl | H | H | H | H | H | Cl | H | cyclopropyl | 2.74[c]; |
| | | | | | | | | | | | 2.12[b] |
| 176 | H | tert-butoxymethyl | H | H | H | H | H | CF₃ | H | cyclobutyl | |
| 177 | H | tert-butoxymethyl | H | H | H | H | H | Cl | H | cyclobutyl | |
| 178 | H | 1,2-dimethoxyethyl | H | H | H | H | H | CF₃ | H | cyclopropyl | |
| 179 | H | 1,2-dimethosyethyl | H | H | H | H | H | Cl | H | cyclobutyl | |
| 180 | H | [2-(2-ethoxy- | H | H | H | H | H | Cl | H | cyclopropyl | 3.63[c]; |
| | | ethoxy)ethyl]sulphanyl | | | | | | | | | 2.58[b] |
| 181 | H | CF₃ | 2-(2-methoxy- | H | H | H | H | Cl | H | cyclopropyl | |
| | | | ethoxy)ethoxy | | | | | | | | |
| 182 | H | CF₃ | 2-(2-methoxy- | H | H | H | H | CF₃ | H | cyclopropyl | 3.79[c]; |
| | | | ethoxy)ethoxy | | | | | | | | 3.52[b] |
| 183 | H | Cl | 2-ethoxyethoxy | H | H | H | H | Cl | H | cyclopropyl | 32.52[b] |
| 184 | H | Cl | 2-ethoxyethoxy | H | H | H | H | CF₃ | H | cyclopropyl | 3.72[b] |
| 185 | H | CF₃ | 2-ethoxyethoxy | H | H | H | H | Cl | H | cyclopropyl | 4[c] |
| | | | | | | | | | | | 2.91[b] |
| 186 | H | CF₃ | 2-ethoxyethoxy | H | H | H | H | CF₃ | H | cyclopropyl | 4.09[b] |
| 187 | H | 1-(butan-2-ylsulphonyl)ethyl | H | H | H | H | H | CF₃ | H | cyclopropyl | 3.34[c]; |
| | | | | | | | | | | | 3.14[b] |
| 188 | H | butan-2-ylsulphanyl | H | H | H | H | H | CF₃ | H | cyclopropyl | 5.01[c]; |
| | | | | | | | | | | | 4.9[b] |
| 189 | H | (2-ethoxyethyl)sulphanyl | H | H | H | H | H | Cl | H | cyclopropyl | 3.76[c]; |
| | | | | | | | | | | | 2.7[b] |
| 190 | H | (2-ethoxyethyl)sulphanyl | H | H | H | H | H | CF₃ | H | cyclopropyl | 4.05[c]; |
| | | | | | | | | | | | 3.87[b] |
| 191 | H | [2-(2-ethoxy- | H | H | H | H | H | CF₃ | H | cyclopropyl | 3.56[c]; |
| | | ethoxy)ethyl]sulphanyl | | | | | | | | | 3.31[b] |
| 192 | H | [2-(2-ethoxy- | H | H | H | H | H | CF₃ | H | cyclopropyl | 3.96[c]; |
| | | ethoxy)ethyl]sulphanyl | | | | | | | | | 3.77[b] |
| 193 | H | 1,2-dimethoxyethyl | H | H | H | H | H | CF₃ | H | cyclobutyl | |
| 194 | H | 1-hydroxy-2-methoxyethyl | H | H | H | H | H | Cl | H | cyclopropyl | 1.26[b] |
| 195 | H | 1-hydroxy-2-methoxyethyl | H | H | H | H | H | CF₃ | H | cyclopropyl | 2.64[c]; |
| | | | | | | | | | | | 1.97[b] |
| 196 | H | 1-(ethylsulphanyl)ethyl | H | H | H | H | H | Cl | H | 2-ethylcyclopropyl | 3.99[b] |
| 197 | H | 1-methoxyethyl | H | H | H | H | H | Cl | H | 2-ethylcyclopropyl | 2.76[b] |
| 198 | H | methoxymethyl | H | H | H | H | H | Cl | H | 3-methylcyclobutyl | 4.04[a] |
| 199 | H | methoxymethyl | H | H | H | H | H | Cl | CH₃ | 2-methylcyclopropyl | 4.36[c]; |
| | | | | | | | | | | | 3.23[b] |
| 200 | H | methoxymethyl | H | H | H | H | H | Br | H | 3-methylcyclobutyl | 2.55[b] |
| 201 | H | (3-methoxypropyl)sulphanyl | H | H | H | H | H | Cl | H | cyclopropyl | 3.77[c]; |
| | | | | | | | | | | | 2.59[b] |
| 202 | H | butylsulphonyl | H | H | H | H | H | CF₃ | H | cyclobutyl | 4.01[c]; |
| | | | | | | | | | | | 3.91[b] |
| 203 | H | butylsulphinyl | H | H | H | H | H | CF₃ | H | cyclobutyl | 3.67[c]; |
| | | | | | | | | | | | 3.4[b] |
| 204 | H | (3-ethoxypropyl)sulphanyl | H | H | H | H | H | Cl | H | cyclopropyl | 4.22[c]; |
| | | | | | | | | | | | 3.02[b] |
| 205 | H | propylsulphinyl | H | H | H | H | H | Cl | H | cyclopropyl | 2.47[c]; |
| | | | | | | | | | | | 1.8[b] |
| 206 | H | propylsulphanyl | H | H | H | H | H | Br | H | 2-methylcyclobutyl | |
| 207 | H | methoxymethyl | H | H | H | H | H | Cl | H | 2-methylcyclobutyl | 4[c]; |
| | | | | | | | | | | | 2.44[b] |
| 208 | H | methoxymethyl | H | H | H | H | H | Cl | H | 2-(trifluoro- | 2.64[b] |
| | | | | | | | | | | methyl)cyclopropyl | |
| 209 | H | 2-methoxyethoxy | H | H | H | H | H | Cl | H | 2-methylcyclopropyl | |
| 210 | H | 2-methoxyethoxy | H | H | H | H | H | Cl | H | 2-trifluoromethyl)- | 2.66[b] |
| | | | | | | | | | | cyclopropyl | |
| 211 | H | 1-methoxyethyl | H | H | H | H | H | Cl | H | 2-(trifluoro- | |
| | | | | | | | | | | methyl)cyclopropyl | |
| 212 | H | 1-methoxyethyl | H | H | H | H | H | Br | H | 3-methylcyclobutyl | 2.84[b] |
| 213 | H | H | (1-methoxypropan- | H | H | H | H | Br | H | 3-methylcyclobutyl | 2.27[b] |
| | | | 2-yl)oxy | | | | | | | | |
| 214 | H | 1-(ethylsulphanyl)ethyl | H | H | H | H | H | Br | H | 3-methycyclobutyl | 3.85[b] |
| 215 | H | 2,2,2-trifluoro-1-(2- | H | H | H | H | H | Cl | H | cyclopropyl | 3.61[c]; |
| | | methoxyethoxy)ethyl | | | | | | | | | 2.63[b] |
| 216 | H | 2,2,2-trifluoro-1-(2- | H | H | H | H | H | CF₃ | H | cyclopropyl | 3.9[c]; |
| | | methoxyethoxy)ethyl | | | | | | | | | 3.72[b] |
| 217 | H | 2,2,2-trifluoro-1-(2- | H | H | H | H | H | Br | H | cyclopropyl | 3.76[c]; |
| | | methoxyethoxy)ethyl | | | | | | | | | 2.85[b] |
| 218 | H | (ethylsulphanyl)methyl | H | H | H | H | H | Br | H | cyclopropyl | 2.79[b] |
| 219 | H | (ethylsulphanyl)methyl | H | H | H | H | H | Cl | H | cyclobutyl | 4.44[c]; |
| | | | | | | | | | | | 3[b] |
| 220 | H | 2,2,2-trifluoro-1-(2- | H | H | H | H | H | CF₃ | H | cyclopropylmethyl | 4.2[c] |
| | | methoxyethoxy)ethyl | | | | | | | | | |
| 221 | H | H | (1-methoxypropan- | H | H | H | H | Cl | H | 2-(trifluoro- | 2.48[b] |
| | | | 2-yl)oxy | | | | | | | methyl)cyclopropyl | |
| 222 | H | (methylsulphonyl)methyl | H | H | H | H | H | Cl | H | 2-(trifluoro- | 1.96[b] |
| | | | | | | | | | | methyl)cyclopropyl | |
| 223 | H | 1-(ethylsulphanyl)ethyl | H | H | H | H | H | Cl | H | 2-(trifluoro- | 3.93[b] |
| | | | | | | | | | | methyl)cyclopropyl | |
| 224 | H | (2,2,2-trifluoro- | H | H | H | H | H | Cl | H | cyclopropyl | 2.56[b] |
| | | ethoxy)methyl | | | | | | | | | |
| 225 | H | methoxyacetyl | H | H | H | H | H | CF₃ | H | cyclopropyl | 2.54[b] |
| 226 | H | 1-methoxyethyl | H | H | H | H | H | Br | H | 2-methylcyclopropyl | 3.98[c]; |
| | | | | | | | | | | | 2.5[b] |
| 227 | H | (1,3-benzodioxol-5- | H | H | H | H | H | CF₃ | H | cyclopropyl | 3.9[b] |
| | | yloxy)methyl | | | | | | | | | |
| 228 | H | 1-methoxyethyl | H | H | H | H | H | Cl | H | 2-methylcyclopropyl | 0.95[b] |
| 229 | H | 1-methoxyethyl | H | H | H | H | H | CF₃ | H | 2-methylcyclopropyl | 4.1[c]; |
| | | | | | | | | | | | 3.61[b] |
| 230 | H | (1,3-benzodioxol-5- | H | H | H | H | H | Cl | H | cyclopropyl | 3.87[c] |
| | | yloxy)methyl | | | | | | | | | |
| 231 | H | (2,2,3,3-tetra- | H | H | H | H | H | CF₃ | H | cyclopropyl | 3.7[b] |
| | | fluoropropoxy)methyl | | | | | | | | | |
| 232 | H | (2,2,3,3-tetra- | H | H | H | H | H | Cl | H | cyclopropyl | 2.69[b] |
| | | fluoropropoxy)methyl | | | | | | | | | |
| 233 | H | ethylsulphanyl | H | H | H | H | H | CF₃ | H | 2-methylcyclobutyl | 5.27[c]; |
| | | | | | | | | | | | 4.89[b] |
| 234 | H | ethylsulphanyl | H | H | H | H | H | CF₃ | H | 3-methylcyclobutyl | 5.34[c]; |
| | | | | | | | | | | | 4.09[b] |
| 235 | H | propylsulphanyl | H | H | H | H | H | CF₃ | H | 2-methylcyclopropyl | 5.23[c]; |
| | | | | | | | | | | | 4.9[b] |
| 236 | H | (2,2-diethoxy- | H | H | H | H | H | CF₃ | H | cyclopropyl | 4.55[c] |
| | | ethyl)sulphanyl | | | | | | | | | |
| 237 | H | (2,2-dimethoxy- | H | H | H | H | H | CF₃ | H | cyclopropyl | 3.37[c] |
| | | ethoxy)methyl | | | | | | | | | |
| 238 | H | (2,2-diethoxyethoxy)methyl | H | H | H | H | H | CF₃ | H | cyclopropyl | 4.09[c] |
| 239 | H | 1,2-dimethoxyethyl | H | H | H | H | H | Br | H | cyclopropyl | |
| 240 | H | 1-ethoxyethyl | H | H | H | H | H | Br | H | 2-methylcyclopropyl | 4.42[c]; |
| | | | | | | | | | | | 2.89[b] |
| 241 | H | 2-(trifluoromethoxy)ethoxy | H | H | H | H | H | Cl | H | cyclopropyl | 2.72[b] |
| 242 | H | ethylsulphinyl | H | H | H | H | H | CF₃ | H | 3-methylcyclobutyl | 3.32[c]; |
| | | | | | | | | | | | 2.98[b] |
| 243 | H | 1-(2-methoxyethoxy)ethyl | H | H | H | H | H | Cl | H | cyclopropyl | 3.26[c]; |
| | | | | | | | | | | | 2[b] |
| 244 | H | ethylsulphinyl | H | H | H | H | H | CF₃ | H | 2-methylcyclobutyl | 3.3[c]; |
| | | | | | | | | | | | 2.9[b] |
| 245 | H | propylsulphonyl | H | H | H | H | H | CF₃ | H | 2-methylcyclopropyl | 3.61[c]; |
| | | | | | | | | | | | 3.43[b] |
| 246 | H | propylsulphinyl | H | H | H | H | H | CF₃ | H | 2-methylcyclopropyl | 3.23[c]; |
| | | | | | | | | | | | 2.9[b] |
| 247 | H | 1,2-dimethoxyethyl | H | H | H | H | H | Br | H | cyclobutyl | |
| 248 | H | (2-ethoxyethoxy)methyl | H | H | H | H | H | Cl | H | cyclopropyl | 2.03[b] |
| 249 | H | tert-butoxymethyl | H | H | H | H | H | Cl | H | cyclopropyl | |
| 250 | H | (2-ethoxyethoxy)methyl | H | H | H | H | H | CF₃ | H | cyclopropyl | 3.17[b] |
| 251 | H | tert-butoxymethyl | H | H | H | H | H | Br | H | cyclopropyl | |
| 252 | H | (2-ethoxyethoxy)methyl | H | H | H | H | H | Cl | H | cyclopropyl | 2.51[b] |
| 253 | H | (2-ethoxyethoxy)methyl | H | H | H | H | H | CF₃ | H | cyclopropyl | 2.73[b] |
| 254 | H | 1-(3-methoxypropoxy)ethyl | H | H | H | H | H | CF₃ | H | cyclopropyl | 3.98[c]; |
| | | | | | | | | | | | 3.49[b] |
| 255 | H | 1-(3-methoxypropoxy)ethyl | H | H | H | H | H | Br | H | cyclopropyl | |
| 256 | H | 1-(3-methoxypropoxy)ethyl | H | H | H | H | H | Cl | H | cyclopropyl | 3.62[c]; |
| | | | | | | | | | | | 2.2[b] |
| 257 | H | 1-(2-methoxyethoxy)ethyl | H | H | H | H | H | CF₃ | H | cyclopropyl | 3.61[c]; |
| | | | | | | | | | | | 3.05[b] |
| 258 | H | 1-ethoxyethyl | H | H | H | H | H | CF₃ | H | cyclopropyl | 4.12[c]; |
| | | | | | | | | | | | 3.6[b] |
| 259 | H | 1-(ethylsulphanyl)ethyl | H | H | H | H | H | Cl | H | 2-methylcyclobutyl | 5.3[c]; |
| | | | | | | | | | | | 3.59[b] |
| 260 | H | tert-butoxymethyl | H | H | H | H | H | CF₃ | H | cyclopropyl | |

The logP values were determined in accordance with EEC Directive 79/831 Annex V.A8 by HPLC (High Performance Liquid Chromatography) on reversed-phase columns (C 18), using the methods below:

[a] The determination is carried out in the acidic range at pH 2.3 using the mobile phases 0.1% aqueous phosphoric acid and acetonitrile, linear gradient from 10% acetonitrile to 95% acetonitrile.

[b] The LC-MS determination in the acidic range is carried out at pH 2.7 using the mobile phases 0.1% aqueous formic acid and acetonitrile (contains 0.1% formic acid), linear gradient from 10% acetonitrile to 95% acetonitrile.

[c] The LC-MS determination in the neutral range is carried out at pH 7.8 using the mobile phases 0.001 molar aqueous ammonium bicarbonate solution and acetonitrile, linear gradient from 10% acetonitrile to 95% acetonitrile.

Calibration is carried out using unbranched alkan-2-ones (having 3 to 16 carbon atoms), with known logP values (determination of the logP values on the basis of the retention times using linear interpolation between two successive alkanones).

The lambda-max values were determined in the maxima of the chromatographic signals using the UV spectra from 200 nm to 400 nm.

| Ex. No. | | |
|---|---|---|
| of Table 1 | ¹H NMR | M + 1 |
| 1 | ¹H NMR (pyrimidine-H): δ = 7.94 | 395.1 |
| 2 | ¹H NMR (pyrimidine-H): δ = 7.94 | 425.1 |
| 3 | ¹H NMR (pyrimidine-H): δ = 8.02 | 393 |
| 4 | ¹H NMR (pyrimidine-H): δ = 7.98 | 429 |
| 5 | ¹H NMR (pyrimidine-H): δ = 7.92 | 349.1 |
| 6 | ¹H NMR (pyrimidine-H): δ = 7.92 | 363.1 |
| 7 | ¹H NMR (pyrimidine-H): δ = 7.90 | 333.1 |
| 8 | ¹H NMR (pyrimidine-H): δ = 7.82 | 303.2 |
| 9 | ¹H NMR (pyrimidine-H): δ = 7.99 | 349.1 |
| 10 | ¹H NMR (pyrimidine-H): δ = 8.01 | 431 |
| 11 | ¹H NMR (pyrimidine-H): δ = 7.91 | 363.1 |
| 12 | ¹H NMR (pyrimidine-H): δ = 7.99 | 407 |
| 13 | ¹H NMR (pyrimidine-H): δ = 7.99 | 393 |
| 14 | ¹H NMR (pyrimidine-H): δ = 7.90 | 349.1 |
| 15 | ¹H NMR (pyrimidine-H): δ = 7.90 | 333.1 |
| 16 | ¹H NMR (pyrimidine-H): δ = 8.16 | 397.2 |
| 17 | ¹H NMR: δ = 9.39 (s, br., 1H), 8.16 (s, 1H), 7.86 (s, br., 1H), | 381.2 |
| | 7.53-7.50 (d, 1H), 7.23 (t, 1H), 6.91-6.89 (d, 1H), 6.19-6.17 (d, | |
| | br., 1H), 4.59-4.53 (m, 1H), 4.27-4.23 (q, 1H), 3.15 (s), | |
| | 2.04-2.00 (m, br., 2H), 1.76-1.65 (m, br., 2H), 1.61-1.54 (m, br., | |
| | 3H), 1.35-1.33 (d, 3H) | |
| 18 | ¹H NMR (pyrimidine-H): δ = 8.16 | 383.1 |
| 19 | ¹H NMR (pyrimidine-H): δ = 8.17 | 367.2 |
| 20 | ¹H NMR (pyrimidine-H): δ = 7.99 | 377.1 |
| 21 | ¹H NMR (pyrimidine-H): δ = 7.93 | 387.1 |
| 22 | ¹H NMR (pyrimidine-H): δ = 7.99 | 329.1 |
| 23 | ¹H NMR (pyrimidine-H): δ = 7.94 | 397.1 |
| 24 | ¹H NMR (pyrimidine-H): δ = 7.88 | 353.1 |
| 25 | ¹H NMR (pyrimidine-H): δ = 8.07 | 421.1 |
| 26 | | 307.1 |
| 28 | ¹H NMR: δ = 9.30 (s, br., 1H), 8.29 (s, br., 1H), 7.94 (s, 1H), | 351.1 |
| | 7.68-7.65 (d, 1H), 7.41 (t, 1H), 7.13-7.11 (d, 1H), 7.04-7.02 (d, | |
| | br., 1H), 4.69-4.59 (m, 1H), 2.98-2.93 (m, 1H), 2.77-2.72 (m, | |
| | 1H), 2.34-2.28 (m, 2H), 2.16-2.10 (m, 2H), 1.74-1.69 (m, 2H), | |
| | 1.09 (t, 3H) | |
| 29 | ¹H NMR (pyrimidine-H): δ = 7.97 | 321.1 |
| 30 | ¹H NMR (pyrimidine-H): δ = 8.05 | 377 |
| 31 | ¹H NMR (pyrimidine-H): δ = 7.91 | 337.1 |
| 32 | ¹H NMR: δ = 9.14 (s, br., 1H), 8.04 (s, 1H), 7.72 (s, br., 1H), | 377 |
| | 7.50-7.46 (d, br., 2H), 7.17 (t, 1H), 4.29-4.20 (m, 2H), 3.67 (s, | |
| | 2H), 2.47-2.41 (q, 2H), 1.17 (t, 3H) | |
| 33 | ¹H NMR (pyrimidine-H): δ = 7.90 | 337.1 |
| 34 | ¹H NMR (pyrimidine-H): δ = 8.22 | 415.2 |
| 35 | ¹H NMR (pyrimidine-H): δ = 8.20 | 399.1 |
| 36 | ¹H NMR (pyrimidine-H): δ = 8.16 | 397.1 |
| 37 | ¹H NMR (pyrimidine-H): δ = 8.16 | 411.1 |
| 38 | | 346.1 |
| 39 | ¹H NMR (pyrimidine-H): δ = 8.17 | 443.1 |
| 40 | ¹H NMR (pyrimidine-H): δ = 8.17 | 429.1 |
| 41 | ¹H NMR (pyrimidine-H): δ = 7.90 | 429.1 |
| 42 | ¹H NMR (pyrimidine-H): δ = 8.17 | 427.1 |
| 43 | ¹H NMR (pyrimidine-H): δ = 8.17 | 413.1 |
| 44 | ¹H NMR (pyrimidine-H): δ = 7.90 | 413.1 |
| 45 | ¹H NMR (pyrimidine-H): δ = 8.18 | 399.1 |
| 46 | ¹H NMR (pyrimidine-H): δ = 8.17 | 415.1 |
| 47 | ¹H NMR (pyrimidine-H): δ = 7.91 | 363.1 |
| 48 | | 362.2 |
| 49 | | 420 |
| 50 | ¹H NMR (methanol-d₄): δ = 8.09-8.08 (d, 1H), 7.82 (s, 1H), | 367.1 |
| | 7.60-7.58 (m, 1H), 7.27-7.26 (m, 2H), 7.24-7.23 (m, 1H), | |
| | 7.03-7.01 (m, 1H), 6.98-6.90 (m, 3H), 5.03 (d, 2H), 2.85-2.80 (m, | |
| | 1H); 0.79-0.75 (m, 2H), 0.62-0.58 (m, 2H) | |
| 51 | ¹H NMR (pyrimidine-H): δ = 8.05 | 304.1 |
| 52 | ¹H NMR (pyrimidine-H): δ = 7.89 | 418 |
| 53 | ¹H NMR: δ = 8.97 (m, 1H), 7.91 (s, 1H), 7.58-7.57 (m, 1H), | 335.1 |
| | 7.38-7.35 (m, 1H), 7.13-7.11 (m, 1H), 6.97 (br. s. 1H), | |
| | 6.49-6.47 (m, 1H), 4.05-4.03 (m, 2H), 3.68-3.62 (m, 2H), | |
| | 3.32-3.30 (s, 3H), 2.87-2.88 (m, 1H), 0.80-0.76 (m, 2H), 0.68.0.66 (m, | |
| | 2H) | |
| 54 | ¹H NMR (pyrimidine-H): δ = 7.99 | 353.1 |
| 55 | ¹H NMR (pyrimidine-H): δ = 8.25 | 387.1 |
| 56 | ¹H NMR (pyrimidine-H): δ = 8.00 | 395.1 |
| 57 | ¹H NMR (pyrimidine-H): δ = 8.00 | 393 |
| 58 | ¹H NMR (pyrimidine-H): δ = 8.00 | 319.2 |
| 59 | ¹H NMR: δ = 8.95 (br. s, 1H), 7.90 (s, 1H), 7.82-7.81 (m, 1H), | 333.1 |
| | 7.77-7.74 (m, 1H) 7.21-7.19 (q, 1H), 6.92-6.90 (m, 2H), 2.98 (s, | |
| | 3H), 2.96-2.92 (m, 1H), 1.43 (s, 6H), 0.80-0.76 (m, 2H), | |
| | 0.68-0.64 (m, 2H) | |
| 60 | ¹H NMR (pyrimidine-H): δ = 7.90 | 319.1 |
| 61 | ¹H NMR (pyrimidine-H): δ = 7.90 | 349.1 |
| 62 | ¹H NMR (pyrimidine-H): δ = 8.16 | 383.2 |
| 63 | ¹H NMR (pyrimidine-H): δ = 7.89 | 349 |
| 64 | ¹H NMR (pyrimidine-H): δ = 7.89 | 347.1 |
| 65 | ¹H NMR (pyrimidine-H): δ = 7.88 | 319 |
| 66 | ¹H NMR (pyrimidine-H): δ = 7.91 | 349.1 |
| 67 | ¹H NMR (pyrimidine-H): δ = 7.90 | 379.1 |
| 68 | ¹H NMR (pyrimidine-H): δ = 7.90 | 369.1 |
| 69 | | |
| 70 | ¹H NMR (pyrimidine-H): δ = 7.99 | 351.1 |
| 71 | ¹H NMR (pyrimidine-H): δ = 8.18 | 383.1 |
| 72 | ¹H NMR (pyrimidine-H): δ = 7.87 | 415.1 |
| 73 | ¹H NMR (pyrimidine-H): δ = 7.85 | 333.2 |
| 74 | ¹H NMR (pyrimidine-H): δ = 7.91 | 365.1 |
| 75 | ¹H NMR (pyrimidine-H): δ = 7.91 | 381.1 |
| 76 | ¹H NMR: δ = 9.02/8.99 (s, br., 1H), 7.92/7.91 (s, 1H), | 379.1 |
| | 7.82/7.77 (s, br., 1H), 7.60-7.58/7.56-7.54 (d, 1H), 7.23 (t, 1H), | |
| | 7.00-6.95 (m, 1H), 6.89-6.87 (d, 1H), 4.65-4.59 (m, 1H), 3.91-3.81 (m, | |
| | 1H), 2.57-2.39 (m), 2.34-2.27 (m, 2H), 2.18-2.13 (m, 2H), | |
| | 1.74-1.65 (m, 2H), 1.59 (t, 3H), 1.15-1.09 (m, 3H) | |
| 77 | ¹H NMR: δ = 9.03 (s, br., 1H), 7.91 (s, 1H), 7.89 (s, br., 1H), | 395.1 |
| | 7.66-7.64 (d, 1H), 7.25 (t, 1H), 7.02-7.00 (m, 1H), 6.98-6.96 (d, | |
| | 1H), 4.67-4.61 (m, 1H), 4.42-4.36 (q, 1H), 2.96-2.85 (m, 2H), | |
| | 2.34-2.25 (m, 2H), 2.20-2.10 (m, 2H), 1.73-1.63 (m, 5H), | |
| | 1.16 (t, 3H) | |
| 78 | ¹H NMR (pyrimidine-H): δ = 8.16 | 417.1 |
| 79 | ¹H NMR (pyrimidine-H): δ = 8.15 | 397.1 |
| 80 | ¹H NMR (pyrimidine-H): δ = 8.16 | 451.2 |
| 81 | ¹H NMR (pyrimidine-H): δ = 8.16 | 387.2 |
| 82 | ¹H NMR (pyrimidine-H): δ = 8.12 | 369.2 |
| 83 | ¹H NMR (pyrimidine-H): δ = 8.04 | 361 |
| 84 | ¹H NMR (pyrimidine-H): δ = 7.98 | 364/366 |
| 85 | ¹H NMR (pyrimidine-H): δ = 7.99 | 307.1 |
| 86 | ¹H NMR (pyrimidine-H): δ = 7.91 | 353-1 |
| 87 | ¹H NMR (pyrimidine-H): δ = 7.91 | 319.1 |
| 88 | ¹H NMR (pyrimidine-H): δ = 8.12 | 383.1 |
| 89 | ¹H NMR (pyrimidine-H): δ = 8.01 | 333.2 |
| 90 | ¹H NMR (pyrimidine-H): δ = 8.08 | 335.1 |
| 91 | ¹H NMR: δ = 9.39 (br. S, 1H), 8.17-8.10 (s, 1H), 7.64-7.63 (m, | 383.1 |
| | 1H), 7.41-7.39 (m, 1H), 7.16-7.12 (m, 1H), 6.81 (br. S., 1H), | |
| | 6.56-6.53 (m, 1H), 4.05-4.02 (m, 2H), 3.68-3.67 (m, 2H), | |
| | 3.56-3.51 (m, 2H), 2.91-2.90 (m 1H), 1.17-1.14 (m, 3H), | |
| | 0.83-0.80 (m, 2H), 0.69-0.66 (m, 2H) | |
| 92 | ¹H NMR (pyrimidine-H): δ = 7.87 | 333.2 |
| 93 | ¹H NMR: δ = 8.97 (br. S, 1H), 7.90 (s, 1H), 7.62-7.60 (m, 1H), | 349.1 |
| | 7.35 (d, 1H) 7.12-7.10 (m, 1H), 6.46 (d, 1H), 4.04-4.03 (m, 2H), | |
| | 3.67-3.65 (m, 2H), 3.49.3.47 (m 2H,), 2.85 (m, 1H), | |
| | 1.14-1.11 (m, 3H), 0.79-0.78 (m, 2H), 0.66.0.65 (m, 2H) | |
| 94 | ¹H NMR: δ = 9.34 (br. s, 1H), 8.07 (s, 1 H), 7.71 (s, 1H), | 351.1 |
| | 7.48 (d, 1H), 7.22 (dd, 2H), 6.88 (d, 1H), 4.37 (s, 2 H), 3.92 (m, 4 | |
| | H), 3.30 (s, 3H), 2.74 (m, 4 H). | |
| 95 | ¹H NMR pyrimidine-H): δ = 7.89 | 293.1 |
| 96 | ¹H NMR (pyrimidine-H): δ = 7.90 | 335.1 |
| 97 | ¹H NMR (pyrimidine-H): δ = 7.98 | 367.1 |
| 98 | ¹H NMR (pyrimidine-H): δ = 7.95 | 351.1 |
| 99 | ¹H NMR (pyrimidine-H): δ = 7.99 | 394/396 |
| 100 | ¹H NMR: δ = 9.30 (s, br., 1H), 8.31 (s, br., 1H), 7.94 (s, 1H), | 365.1 |
| | 7.67-7.64 (m, 1H), 7.41 (t, 1H), 7.15-7.12 (m, 1H), | |
| | 7.04-7.02 (d, br., 1H), 4.67-4.60 (m, 1H), 2.87-2.82 (m, 1H), | |
| | 2.78-2.72 (m, 1H), 2.33-2.30 (m, 2H), 2.14-2.12 (m, 2H), 1.74-1.68 (m, | |
| | 4H), 0.98 (t, 3H) | |
| 101 | ¹H NMR (pyrimidine-H): δ = 8.18 | 355.1 |
| 102 | ¹H NMR (pyrimidine-H): δ = 8.02 | 395.1 |
| 103 | ¹H NMR (pyrimidine-H): δ = 7.96 | 381.1 |
| 104 | ¹H NMR (pyrimidine-H): δ = 8.00 | 407.1 |
| 105 | ¹H NMR (pyrimidine-H): δ = 8.00 | 393 |
| 106 | ¹H NMR (pyrimidine-H): δ = 7.92 | 363.2 |
| 107 | ¹H NMR (pyrimidine-H): δ = 8.18 | 383.1 |
| 108 | ¹H NMR: δ = 8.95 (br. S, 1H), 7.66 (s, 1H), 7.81 (m, 1H), | 333.2 |
| | 7.69-7.67 (m, 1H), 7.18-7.16 (m, 1H), 6.88-6.83 (m, 2H), 4.34 (s, | |
| | 2H), 3.56 (s, 3H) 3.29-3.27 (q, 3H), 2.70 (m, 1H), 1.48-1.46 (m, | |
| | 1H), 1.21-1.77 (m, 2H), 0.96-0.92 (m, 2H), 0.78-0.77 (m, 1H), | |
| | 0.6 (m, 1H) | |
| 109 | ¹H NMR (pyrimidine-H): δ = 8.18 | 369.1 |
| 110 | ¹H NMR: δ = 9.12 (s, br., 1H), 8.42 (s, 1H), 7.51-7.48 (m, 2H), | 411.1 |
| | 7.35 (t, 1H), 7.12-7.10 (d, 1H), 4.34-4.30 (m, 1H), 3.16 (s), | |
| | 3.02-2.95 (m, 1H), 1.39-1.33 (m, 3H), 0.85-0.80 (m, 2H), | |
| | 0.69-0.66 (m, 2H) | |
| 111 | ¹H NMR (pyrimidine-H): δ = 8.05 | 411.1 |
| 112 | ¹H NMR (pyrimidine-H): δ = 8.03 | 395.1 |
| 113 | ¹H NMR (pyrimidine-H): δ = 7.96 | 353.1 |
| 114 | ¹H NMR (pyrimidine-H): δ = 7.94 | 337.1 |
| 115 | ¹H NMR (pyrimidine-H): δ = 7.90 | 363.1 |
| 116 | ¹H NMR: δ = 9.88 (s, br., 1H), 8.64 (s, br., 1H), 8.22 (s, 1H), | 387.1 |
| | 8.07-8.04 (m, 1H), 7.54 (t, 1H), 7.45-7.43 (d, 1H), 6.94 (s, br., | |
| | 1H), 3.22-3.17 (q, 2H), 3.08 (s), 3.02-2.98 (m, 1H), | |
| | 1.14-1.10 (m, 4H), 0.88-0.79 (m, 2H), 0.68-0.64 (m, 2H) | |
| 117 | ¹H NMR: δ = 8.37 (s, br., 1H), 8.20 (s, 1H), 7.88-7.85 (m, 1H), | 371.1 |
| | 7.45 (t, 1H), 7.19-7.17 (d, 1H), 6.89 (s, br., 1H), 3.00-2.88 (m, | |
| | 2H), 2.76-2.67 (m, 1H), 1.05 (t, 3H), 0.88-0.80 (m, 2H), | |
| | 0.68-0.66 (m, 2H) | |
| 118 | ¹H NMR (pyrimidine-H): δ = 8.22 | 401.1 |
| 119 | ¹H NMR: δ = 9.83 (s, br., 1H), 8.62 (s, br., 1H), 8.22 (s, 1H), | 385.1 |
| | 7.87-7.84 (m, 1H), 7.55 (t, 1H), 7.47-7.45 (m, 1H), | |
| | 6.83-6.81 (d, br., 1H), 4.78-4.72 (m, 1H), 3.26-3.21 (q, 2H), 2.35-2.28 (m, | |
| | 2H), 2.20-2.10 (m, 2H), 1.80-1.68 (m, 2H), 1.16 (t, 3H) | |
| 120 | ¹H NMR (pyrimidine-H): δ = 7.91 | 395.1 |
| 121 | ¹H NMR (pyrimidine-H): δ = 8.22 | 415.2 |
| 122 | ¹H NMR (pyrimidine-H): δ = 8.20 | 399.2 |
| 123 | ¹H NMR (pyrimidine-H): δ = 7.96 | 381.1 |
| 124 | ¹H NMR (pyrimidine-H): δ = 7.94 | 365.1 |
| 125 | ¹H NMR (pyrimidine-H): δ = 8.04 | 425.1 |
| 126 | ¹H NMR (pyrimidine-H): δ = 8.03 | 409.1 |
| 127 | ¹H NMR (pyrimidine-H): δ = 8.16 | 353.2 |
| 128 | ¹H NMR (pyrimidine-H): δ = 8.18 | 397.2 |
| 129 | ¹H NMR: δ = 8.94 (s, br., 1H), 7.90 (s, 1H), 7.82 (s, br., 1H), | 321.2 |
| | 7.49-7.47 (d, 1H), 7.19 (t, 1H), 6.84-6.82 (d, 1H), 6.46-6.44 (d, | |
| | br., 1H), 4.39-4.34 (m, 1H), 4.26-4.21 (q, 1H), 3.14 (s, 3H), | |
| | 1.34-1.33 (d, 3H), 1.25-1.23 (d, 6H) | |
| 130 | ¹H NMR: δ = 9.05 (s, br., 1H), 7.99 (s, 1H), 7.71-7.68 (d, 1H), | 381.1 |
| | 7.55-7.50 (m, 1H), 7.20 (t, 1H), 6.86-6.84 (d, 1H), 6.22-6.20 (d, | |
| | br., 1H), 4.34-4.23 (m, 2H), 3.15 (s, 3H), 1.39-1.37 (d, 3H), | |
| | 1.34-1.33 (d, 3H), 1.31-1.19 (d, 1H), 1.06-0.99 (d, 3H) | |
| 131 | ¹H NMR (pyrimidine-H): δ = 7.90 | 351.2 |
| 132 | ¹H NMR (pyrimidine-H): δ = 8.17 | 411.1 |
| 133 | ¹H NMR: δ = 8.99 (s, br., 1H), 7.90 (s, 1H), 7.89-7.88 (m, 1H), | 377.1 |
| | 7.66-7.62 (m, 1H), 7.16 (t, 1H), 6.95 (s, br., 1H), 6.89-6.88 (d, | |
| | 1H), 3.97-3.95 (m, 1H), 3.56 (s, 2H), 2.99-2.94 (m, 1H), | |
| | 2.56-2.45 (m), 1.53-1.36 (m, 6H), 1.18-1.16 (d, 3H), 1.08-1.07 (d, | |
| | 3H), 0.87-0.77 (m, 8H), 0.69-0.66 (m, 2H) | |
| 134 | ¹H NMR (pyrimidine-H): δ = 7.89 | 391.1 |
| 135 | ¹H NMR (pyrimidine-H): δ = 8.16 | 425.2 |
| 136 | | 347.4 |
| 137 | ¹H NMR (pyrimidine-H): δ = 8.02 | 333.1 |
| 138 | ¹H NMR: δ = 9.01 (br. s, 1H), 7.94 (s, 1 H), 7.75 (s, 1H), | 311.1 |
| | 7.51 (dd, 1H), 7.20 (dd, 1H), 7.08 (t, 1H), 6.85 (dd, 1H), 4.60 (dt, | |
| | 2H), 4.36 (s, 2H), 3.74 (m, 2 H), 3.29 (s 3H) | |
| 139 | | 347.2 |
| 140 | | 335.4 |
| 141 | | 321.4 |
| 142 | | 335.4 |
| 143 | | 335.4 |
| 144 | | 335.4 |
| 145 | | 335.4 |
| 146 | | 335.4 |
| 147 | | 335.4 |
| 148 | | 321.4 |
| 149 | | 307.4 |
| 150 | ¹H NMR (pyrimidine-H): δ = 7.90 | 383.1 |
| 151 | ¹H NMR (pyrimidine-H): δ = 8.05 | 439 |
| 152 | ¹H NMR (pyrimidine-H): δ = 8.03 | 423 |
| 153 | ¹H NMR: δ = 9.05 (s, br., 1H), 8.00 (s, 1H), 7.88 (s, br., 1H), | 439 |
| | 7.66-7.64 (d, 1H), 7.25 (t, 1H), 7.02-7.00 (d, 1H), 6.71-6.69 (d, | |
| | 1H), 4.67-4.61 (m, 1H), 4.42-4.36 (q, 1H), 2.96-2.85 (m, 2H), | |
| | 2.33-2.26 (m, 2H), 2.18-2.10 (m, 2H), 1.73-1.62 (m, 5H), | |
| | 1.20-1.14 (m, 3H) | |
| 154 | ¹H NMR (pyrimidine-H): δ = 7.99 | 425 |
| 155 | ¹H NMR (pyrimidine-H): δ = 8.18 | 383.1 |
| 156 | ¹H NMR (pyrimidine-H): δ = 8.16 | 447.1 |
| 157 | ¹H NMR (pyrimidine-H): δ = 8.04 | 383.1 |
| 158 | ¹H NMR (pyrimidine-H): δ = 7.89 | 383 |
| 159 | ¹H NMR (pyrimidine-H): δ = 8.05 | 425 |
| 160 | ¹H NMR (pyrimidine-H): δ = 8.17 | 427.2 |
| 161 | ¹H NMR (pyrimidine-H): δ = 7.96 | 381.1 |
| 162 | ¹H NMR (pyrimidine-H): δ = 7.94 | 365.1 |
| 163 | ¹H NMR: δ = 9.09 (s, br., 1H), 7.91 (s, 1H), 7.88-7.83 (m, 2H), | 409.1 |
| | 7.24 (t, 1H), 7.01-6.96 (d, 2H), 4.47-4.42 (m, 1H), | |
| | 2.99-2.94 (m, 1H), 2.88-2.81 (m, 1H), 1.96-1.62 (m, 1H), 1.61-1.59 (m, | |
| | 3H), 1.41-1.38 (m, 1H), 1.23-1.21 (d, 1H), 1.15-1.14 (d, 1H), | |
| | 0.92-0.87 (m, 3H), 0.78-0.74 (m, 2H), 0.68-0.64 (m, 2H) | |
| 164 | ¹H NMR: δ = 9.08 (s, br., 1H), 7.91 (s, 1H), 7.81-7.75 (m, 2H), | 393.1 |
| | 7.25-7.20 (m, 1H), 6.99-6.98 (m, 1H), 6.87-6.84 (m, 1H), | |
| | 3.87-3.79 (m, 1H), 2.95-2.93 (m, 1H), 2.33-2.25 (m, 1H), | |
| | 1.60-1.47 (m, 4H), 1.45-1.38 (m, 1H), 1.11-1.09 (d, 2H), 1.05-1.02 (m, | |
| | 1H), 0.94-0.90 (m, 1H), 0.82-0.76 (m, 3H), 0.68-0.66 (m, 2H) | |
| 165 | ¹H NMR: δ = 9.09 (s, br., 1H), 7.90 (s, 1H), 7.88-7.82 (m, 2H), | 423.1 |
| | 7.24 (t, 1H), 7.02-6.96 (m, 2H), 4.48-4.41 (m, 1H), | |
| | 2.99-2.85 (m, 2H), 1.90-1.65 (m, 2H), 1.61-1.59 (m, 3H), 1.45-1.30 (m, | |
| | 3H), 1.25-1.05 (d, 1H), 0.85-0.76 (m, 5H), 0.67-0.65 (m, 2H) | |
| 166 | ¹H NMR: δ = 9.07 (s, br., 1H), 7.91 (s, 1H), 7.81-7.75 (m, 2H), | 407.1 |
| | 7.25-7.20 (m, 1H), 6.99-6.98 (m, 1H), 6.87-6.84 (m, 1H), | |
| | 4.40-4.25 (m, 1H), 2.95-2.90 (m, 1H), 2.42-2.35 (m, 1H), | |
| | 1.55-1.40 (m, 4H), 1.35-1.15 (m, 3H), 1.10-1.03 (d, 4H), 0.88-0.66 (m, | |
| | 7H) | |
| 167 | ¹H NMR (pyrimidine-H): δ = 8.17 | 441.1 |
| 168 | ¹H NMR (pyrimidine-H): δ = 8.17 | 457.1 |
| 170 | ¹H NMR (pyrimidine-H): δ = 7.96 | 395.1 |
| 171 | ¹H NMR (pyrimidine-H): δ = 7.94 | 379.1 |
| 172 | ¹H NMR (pyrimidine-H): δ = 7.99 | 279.1 |
| 173 | ¹H NMR (pyrimidine-H): δ = 7.89 | 413.1 |
| 174 | ¹H NMR (pyrimidine-H): δ = 7.76 | 349.1 |
| 175 | ¹H NMR (pyrimidine-H): δ = 7.94 | 365.1 |
| 180 | ¹H NMR (pyrimidine-H): δ = 7.91 | 409.1 |
| 181 | ¹H NMR (pyrimidine-H): δ = 7.90 | 447.1 |
| 182 | ¹H NMR (pyrimidine-H): δ = 8.16 | 481.1 |
| 183 | ¹H NMR (pyrimidine-H): δ = 7.89 | 383.1 |
| 184 | ¹H NMR: δ = 9.47 (br. s. 1H), 8.20 (d, 1H), 8.16 (d, 1H), | 417.1 |
| | 7.61-7.58 (q, 1H), 7.09-7.07 (q, 1H), 6.87-6.82 (m, 1H), | |
| | 4.13-4.11 (m, 2H), 3.71-3.69 (m, 2H), 3.54 (m, 2H), 2.85-2.82 (m, 1H), | |
| | 1.14-1.11 (m, 3H), 0.86-0.85 (m, 2H), 0.69-0.67 (m, 2H) | |
| 185 | ¹H NMR (pyrimidine-H): δ = 7.91 | 417.1 |
| 186 | ¹H NMR (pyrimidine-H): δ = 8.16 | 451.1 |
| 187 | ¹H NMR (pyrimidine-H): δ = 8.17 | 443.1 |
| 188 | ¹H NMR (pyrimidine-H): δ = 8.18 | 383.1 |
| 189 | ¹H NMR (pyrimidine-H): δ = 7.92 | 365.1 |
| 190 | ¹H NMR (pyrimidine-H): δ = 8.18 | 399.1 |
| 191 | ¹H NMR (pyrimidine-H): δ = 8.18 | 429.1 |
| 192 | ¹H NMR (pyrimidine-H): δ = 8.18 | 443.1 |
| 194 | ¹H NMR (pyrimidine-H): δ = 7.88 | 335.1 |
| 195 | ¹H NMR (pyrimidine-H): δ = 8.16 | 369.1 |
| 196 | | 376.1 |
| 197 | | 346.1 |
| 198 | ¹H NMR (pyrimidine-H): δ = 7.89 | 333 |
| 199 | ¹H NMR (pyrimidine-H): δ = 7.99 | 333 |
| 200 | ¹H NMR (pyrimidine-H): δ = 7.98 | 378/380 |
| 201 | ¹H NMR: δ = 9.08 (s, br., 1H), 7.99-7.98 (m, 1H), 7.91 (s, 1H), | 365.1 |
| | 7.61-7.59 (m, 1H), 7.17 (t, 1H), 7.03 (s, br., 1H), 6.87-6.85 (d, | |
| | 2H), 3.40 (t, 2H), 3.22 (s, 3H), 2.93 (t, 2H), 2.87-2.84 (m, 1H), | |
| | 1.83-1.76 (m, 2H), 0.84-0.80 (m, 2H), 0.68-0.66 (m, 2H) | |
| 202 | ¹H NMR (pyrimidine-H): δ = 8.22 | 429.2 |
| 203 | ¹H NMR (pyrimidine-H): δ = 8.20 | 413.2 |
| 204 | ¹H NMR (pyrimidine-H): δ = 7.91 | 379.2 |
| 205 | ¹H NMR (pyrimidine-H): δ = 7.94 | 351 |
| 206 | ¹H NMR (pyrimidine-H): δ = 7.88 | 408/410 |
| 207 | ¹H NMR (pyrimidine-H): δ = 7.91 | 333 |
| 208 | ¹H NMR (pyrimidine-H): δ = 7.96 | 373.1 |
| 209 | ¹H NMR (pyrimidine-H): δ = 7.98 | 349.1 |
| 210 | ¹H NMR (pyrimidine-H): δ = 7.97 | 403.1 |
| 211 | ¹H NMR (pyrimidine-H): δ = 7.96 | 387.1 |
| 212 | | 392/394 |
| 213 | | 422/424 |
| 214 | | 422/424 |
| 215 | ¹H NMR (pyrimidine-H): δ = 7.91 | 417.1 |
| 216 | ¹H NMR (pyrimidine-H): δ = 8.18 | 451.2 |
| 217 | ¹H NMR (pyrimidine-H): δ = 8.00 | 461 |
| 218 | ¹H NMR (pyrimidine-H): δ = 7.99 | 379 |
| 219 | ¹H NMR (pyrimidine-H): δ = 7.91 | 349.1 |
| 220 | ¹H NMR (pyrimidine-H): δ = 8.17 | 465.2 |
| 221 | ¹H NMR (pyrimidine-H): δ = 7.94 | |
| 222 | ¹H NMR (pyrimidine-H): δ = 7.94 | |
| 223 | ¹H NMR (pyrimidine-H): δ = 7.96 | 417.1 |
| 224 | ¹H NMR (pyrimidine-H): δ = 7.91 | 373.1 |
| 225 | ¹H NMR (pyrimidine-H): δ = 8.19 | 367.1 |
| 226 | ¹H NMR (pyrimidine-H): δ = 7.99 | 378/380 |
| 227 | ¹H NMR (pyrimidine-H): δ = 8.17 | |
| 228 | ¹H NMR: δ = 8.96 (s, 1 H), 7.87 (s, 1 H), 7.73 (d, 2 H), | 333 |
| | 7.18 (dd, 1 H), 6.89 (s, 1 H), 6.82 (d, 1 H), 4.25 (m, 1 H), 3.13 (s, 3 | |
| | H), 2.60-2.68 (m, 1 H), 1.33 (d, 3 H), 1.10 (d, 3 H), | |
| | 1.01-1.05 (m, 1 H), 0.78-0.83 (m, 1 H), 0.57-0.61 (m, 1 H) | |
| 229 | ¹H NMR (pyrimidine-H): δ = 8.16 | 367 |
| 230 | ¹H NMR (pyrimidine-H): δ = 7.90 | |
| 231 | ¹H NMR (pyrimidine-H): δ = 8.17 | |
| 232 | ¹H NMR (pyrimidine-H): δ = 7.91 | |
| 233 | ¹H NMR (pyrimidine-H): δ = 8.16 | 383 |
| 234 | ¹H NMR (pyrimidine-H): δ = 8.17 | 383 |
| 235 | ¹H NMR (pyrimidine-H): δ = 8.16 | 383 |
| 236 | ¹H NMR (pyrimidine-H): δ = 8.18 | |
| 237 | ¹H NMR (pyrimidine-H): δ = 8.17 | |
| 238 | ¹H NMR (pyrimidine-H): δ = 8.17 | |
| 240 | ¹H NMR (pyrimidine-H): δ = 7.99 | 392/394 |
| 241 | ¹H NMR (pyrimidine-H): δ = 7.93 | 389.1 |
| 242 | ¹H NMR (pyrimidine-H): δ = 8.21 | 399 |
| 243 | ¹H NMR: δ = 9.24 (s, br., 1H), 7.93 (s, 2H), 7.69-7.67 (m, 1H), | 363.2 |
| | 7.23-7.18 (m, 2H), 6.85-6.83 (d, 1H), 4.38-4.33 (m, 1H), | |
| | 3.42-3.22 (m), 2.91-2.86 (m, 1H), 1.33-1.31 (d, 3H), 0.82-0.78 (m, | |
| | 2H), 0.68-0.66 (m, 2H) | |
| 244 | ¹H NMR (pyrimidine-H): δ = 8.22 | 399 |
| 245 | ¹H NMR (pyrimidine-H): δ = 8.23 | 415 |
| 246 | ¹H NMR (pyrimidine-H): δ = 8.22 | 399 |
| 248 | ¹H NMR (pyrimidine-H): δ = 7.93 | |
| 250 | ¹H NMR (pyrimidine-H): δ = 8.19 | |
| 252 | ¹H NMR (pyrimidine-H): δ = 7.93 | |
| 253 | ¹H NMR (pyrimidine-H): δ = 8.19 | |
| 254 | ¹H NMR: δ = 9.66 (s, br., 1H), 8.19 (s, 1H), 7.95 (s, br., 1H), | 411.2 |
| | 7.74-7.72 (d, 1H), 7.24 (t, 1H), 7.09 (s, br., 1H), 6.90-6.88 (d, | |
| | 1H), 4.34-4.30 (m, 1H), 3.40-3.30 (m), 3.21 (s), 3.17 (s), | |
| | 2.93-2.91 (m, 1H), 1.74-1.65 (m, 4H), 1.32-1.30 (d, 3H), | |
| | 0.84-0.82 (m, 2H), 0.70-0.68 (m, 2H) | |
| 255 | ¹H NMR (pyrimidine-H): δ = 8.01 | 421 |
| 256 | ¹H NMR: δ = 9.24 (s, br., 1H), 7.93 (s, 2H), 7.67-7.65 (d, 1H), | 377.2 |
| | 7.23-7.18 (m, 2H), 6.83-6.81 (d, 1H), 4.33-4.28 (m, 1H), | |
| | 3.37-3.24 (m), 3.17 (s, 3H), 2.90-2.86 (m, 1H), 1.71-1.65 (m, 2H), | |
| | 1.31-1.30 (d, 3H), 0.82-0.78 (m, 2H), 0.68-0.66 (m, 2H) | |
| 257 | ¹H NMR: δ = 9.65 (s, br., 1H), 8.19 (s, 1H), 7.94 (s, br., 1H), | 397.2 |
| | 7.74-7.72 (d, 1H), 7.24 (t, 1H), 7.09 (s, br., 1H), 6.91-6.89 (d, | |
| | 1H), 4.40-4.37 (m, 1H), 3.60-3.30 (m), 3.25 (s), 3.22 (s), | |
| | 2.93-2.90 (m, 1H), 1.33-1.31 (d, 3H), 0.84-0.81 (m, 2H), | |
| | 0.69-0.67 (m, 2H) | |
| 258 | ¹H NMR: δ = 9.65 (s, br., 1H), 8.19 (s, 1H), 7.96 (s, br., 1H), | 367.2 |
| | 7.73-7.71 (d, 1H), 7.23 (t, 1H), 7.10 (s, br., 1H), 6.90-6.88 (d, | |
| | 1H), 4.36-4.31 (q, 1H), 3.31-3.20 (m), 2.94-2.88 (m, 1H), | |
| | 1.31-1.30 (d, 3H), 1.07 (t, 3H), 0.86-0.80 (m, 2H), 0.69-0.66 (m, 2H) | |
| 259 | ¹H NMR (pyrimidine-H): δ = 7.93 | 377 |

The chemical NMR shifts were measured in ppm at 400 MHz, unless indicated otherwise, in the solvent DMSO-d₆ using tetramethylsilane as the internal standard.

The following abbreviations describe the signal splitting:

s=singlet, d=doublet, t=triplet, q=quadruplet, m=multiplet

### Example A

Venturia Test (Apple)/Protective
Solvents: 24.5 parts by weight of acetone 24.5 parts by weight of dimethylacetamide
Emulsifier: 1 part by weight of alkylaryl polyglycol ether

To prepare a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and then remain for 1 day in an incubation cabinet at about 20° C. and 100% relative atmospheric humidity.

The plants are then placed in a greenhouse at about 21° C. and a relative atmospheric humidity of about 90%.

Evaluation is carried out 10 ten days after the inoculation. 0% means an efficacy which corresponds to that of the control, whereas an efficacy of 100% means that no infection is observed.

In this test, the Examples 1, 11, 12, 13, 14, 15, 20, 26, 27, 28, 33, 35, 52, 53, 55, 56, 57, 58, 59, 60, 61, 62, 63, 64, 65, 66, 76, 77, 81, 83, 92, 93, 100, 106, 111, 113, 116, 118, 119, 120, 121, 125, 129, 133, 138, 153, 163, 164, 165, 166, 171, 205, 211, 216 and 217 of Table I show, at an active compound concentration of 100 ppm, an efficacy of 70% or more.

### Example B

Alternaria Test (Tomato)/Protective
Solvent: 24.5 parts by weight of acetone 24.5 parts by weight of dimethylacetamide
Emulsifier: 1 part by weight of alkylaryl polyglycol ether

To prepare a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of Alternaria solani. The plants are then placed in an incubation cabinet at about 20° C. and 100% relative atmospheric humidity.

Evaluation is carried out 3 days after the inoculation. 0% means an efficacy which corresponds to that of the control, whereas an efficacy of 100% means that no infection is observed.

In this test, the Examples 11, 12, 13, 14, 15, 20, 26, 27, 28, 33, 35, 52, 55, 57, 59, 60, 61, 62, 63, 64, 65, 66, 55, 76, 77, 81, 83, 92, 93, 100, 106, 111, 113, 118, 119, 120, 121, 125, 129, 133, 134, 135, 137, 138, 153, 162, 163, 164, 165, 166, 171, 205, 216 and 217 of Table I show, at an active compound concentration of 100 ppm, an efficacy of 70% or more.

### Example C

Sphaerotheca Test (Cucumber)/Protective
Solvent: 49 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the preparation of active compound at the stated application rate. 1 day after the treatment, the plants are inoculated with a spore suspension of Sphaerotheca fuliginea. The plants are subsequently placed in a greenhouse at 70% relative atmospheric humidity and a temperature of 23° C.

Evaluation is carried out 7 days after the inoculation. 0% means an efficacy which corresponds to that of the control, whereas an efficacy of 100% means that no infection is observed.

In this test, the Examples Nos. 8, 10, 11, 13, 15, 18, 21, 22, 27, 28, 30, 31, 32, 33, 34, 35, 36, 39, 40, 41, 42, 43, 44, 45, 46, 48, 53, 57, 58, 59, 60, 61, 62, 67, 73, 75, 76, 77, 79, 83, 85, 91, 92, 93, 98, 101, 109, 111, 112, 113, 114, 115, 116, 117, 118, 119, 120, 121, 124, 127, 129, 131, 132, 133, 134, 135, 137, 138, 148, 149, 150, 152, 153, 154, 160, 163, 164, 165, 166, 167, 175, 187, 190, 194, 195, 198, 199, 203, 205, 218, 219, 224, 225, 226, 227 and 228 of Table I show, at an active compound concentration of 500 ppm, an efficacy of 70% or more.

### Example D

Leptosphaeria Nodorum Test (Wheat)/Protective
Solvent: 49 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young wheat plants are sprayed with the preparation of active compound at the stated application rate. 1 day after the treatment, the plants are inoculated with an aqueous spore suspension of Leptosphaeria nodorum and then remain for 48 h at 100% relative atmospheric humidity and 22° C. Thereafter, the plants are placed in a greenhouse at 90% relative atmospheric humidity and a temperature of 22° C.

Evaluation is carried out 7-9 days after the inoculation. 0% means an efficacy which corresponds to that of the control, whereas an efficacy of 100% means that no infection is observed.

In this test, the Examples Nos. 1, 2, 4, 5, 6, 7, 8, 9, 10, 11, 12, 13, 14, 15, 16, 17, 18, 19, 20, 21, 22, 23, 24, 26, 27, 28, 29, 31, 33, 34, 35, 36, 37, 39, 40, 41, 42, 43, 48, 50, 53, 56, 57, 58, 59, 60, 61, 62, 63, 64, 65, 66, 67, 68, 70, 71, 72, 73, 74, 75, 76, 77, 78, 79, 81, 82, 83, 84, 85, 86, 88, 90, 91, 92, 93, 95, 96, 97, 98, 99, 100, 101, 102, 103, 104, 105, 106, 107, 108, 109, 110, 111, 112, 113, 114, 115, 116, 117, 118, 119, 120, 121, 123, 124, 125, 126, 127, 128, 129, 131, 132, 133, 134, 135, 136, 137, 138, 142, 144, 148, 149, 150, 151, 152, 153, 154, 155, 156, 158, 159, 160, 161, 162, 163, 164, 165, 166, 167, 168, 170, 171, 175, 180, 181, 187, 188, 189, 190, 191, 192, 194, 195, 196, 197, 198, 199, 200, 201, 202, 203, 204, 205, 207, 208, 209, 210, 211, 212, 213, 214, 215, 216, 217, 218, 219, 223, 224, 225, 226, 227, 228, 229, 231, 232, 234, 235, 236, 237 and 240 of Table I showed, at an active compound concentration of 500 ppm, an efficacy of 70% or more.

### Example E

Pyrenophora Teres Test (Barley)/Protective
Solvent: 50 parts by weight of N,N-dimethylacetamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are sprayed with a spore suspension of Pyrenophora teres. The plants remain in an incubation cabinet for 48 hours at 20° C. and 100% relative atmospheric humidity. The plants are placed in a greenhouse at 80% relative atmospheric humidity and a temperature of about 20° C.

Evaluation is carried out 8 days after the inoculation. 0% means an efficacy which corresponds to that of the control, whereas an efficacy of 100% means that no infection is observed.

In this test, the Examples Nos. 5, 11, 12, 13, 14, 15, 19, 20, 24, 28, 33, 45, 53, 55, 56, 57, 58, 59, 60, 64, 66, 75, 76, 77, 81, 82, 88, 91, 93, 97, 98, 101, 102, 112, 113, 114, 116, 117, 118, 119, 124, 126, 127, 131, 132, 133, 138, 153, 154, 163, 164, 166, 175, 180, 190, 191, 194, 195, 204, 205 and 209 of Table I showed, at an active compound concentration of 500 ppm, an efficacy of 70% or more.

### Example F

Pyricularia Test (Rice)/Protective
Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with an aqueous spore suspension of Pyricularia oryzae. The plants are then placed in a greenhouse at 100% relative atmospheric humidity and 25° C.

Evaluation is carried out 5 days after the inoculation. 0% means an efficacy which corresponds to that of the control, whereas an efficacy of 100% means that no infection is observed.

In this test, Examples Nos. 5, 6, 11, 13, 14, 15, 19, 20, 21, 24, 26, 27, 28, 53, 56, 57, 59, 60, 61, 62, 64, 66, 70, 71, 73, 75, 76, 77, 79, 81, 82, 83, 84, 86, 88, 91, 92, 93, 95, 96, 97, 98, 99, 100, 101, 102, 105, 106, 107, 108, 109, 116, 117, 121, 126, 127, 129, 138, 151, 152, 153, 154, 165, 166, 195, 201 and 207 of Table I showed, at an active compound concentration of 250 ppm, an efficacy of 80% or more.

### Example G

Rhizoctonia Test (Rice)/Protective
Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with hypha of Rhizoctonia solani. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, Examples Nos. 4, 5, 6, 7, 11, 14, 15, 19, 20, 24, 26, 28, 31, 34, 53, 57, 59, 60, 61, 62, 70, 71, 76, 77, 79, 81, 83, 86, 88, 91, 100, 101, 102, 103, 105, 106, 107, 109, 116, 117, 121, 126, 127, 129, 151, 152, 153, 154, 165, 166, 195, 201 and 207 of Table I showed, at an active compound concentration of 250 ppm, an efficacy of 80% or more.

### Example H

Cochliobolus Test (Rice)/Protective
Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with an aqueous spore suspension of Cochliobolus miyabeanus. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, Examples Nos. 5, 7, 11, 15, 19, 20, 26, 27, 28, 53, 56, 57, 59, 60, 62, 70, 71, 75, 76, 77, 81, 82, 83, 84, 88, 91, 92, 93, 95, 99, 100, 101, 102, 103, 105, 106, 107, 108, 109, 116, 117, 121, 126, 127, 129, 152, 153, 154, 165, 166 and 201 of Table I showed, at an active compound concentration of 250 ppm, an efficacy of 80% or more.

### Example I

Gibberella Test (Rice)/Protective
Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with an aqueous spore suspension of Gibberella zeae. The plants are then placed in a greenhouse at 100% relative atmospheric humidity and 25° C.

Evaluation is carried out 5 days after the inoculation. 0% means an efficacy which corresponds to that of the control, whereas an efficacy of 100% means that no infection is observed.

In this test, Example Nos. 6, 26, 73, 129 and 138 of Table I showed, at an active compound concentration of 250 ppm, an efficacy of 80% or more.

### Example J

Phakopsora Test (Soya Beans)/Protective
Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young plants are sprayed with the preparation of active compound at the stated application rate. 1 day after the treatment, the plants are inoculated with an aqueous spore suspension of Phakopsora pachyrhizi. The plants are then placed in a greenhouse at 80% relative atmospheric humidity and 20° C.

Evaluation is carried out 1 day after the inoculation. 0% means an efficacy which corresponds to that of the control, whereas an efficacy of 100% means that no infection is observed.

In this test, compounds Nos. 11, 135, 163 and 165 of Table I showed, at an active compound concentration of 250 ppm, an efficacy of 80% or more.

### Example K

Production of Fumonisin FB1 by Fusarium Proliferatum

The method used was adapted to microtitre plates using the method described by Lopez-Errasquin et al.: Journal of Microbiological Methods 68 (2007) 312-317.

Fumonisin-inducing liquid medium (Jimenez et al., Int. J. Food Microbiol. (2003), 89, 185-193) was inoculated with a concentrated spore suspension of Fusarium proliferatum (350 000 spores/ml, stored at −160° C.) to a final concentration of 2000 spores/ml.

The compounds were dissolved (10 mM in 100% DMSO) and diluted to 100 μM in H₂O. The compounds were tested at 7 concentrations in a range of from 50 μM to 0.01 μM (diluted, starting with the 100 μM stock solution in 10% DMSO).

From each diluted solution, 5 μl were mixed with 95 μl of inoculated medium in a well of a 96-well microarray plate. The plate was covered and incubated at 20° C. for 6 days.

At the beginning and after 6 days, an OD measurement (OD620 multiple read per well (square: 3×3)) was carried out to calculate the “pI50” growth.

After 6 days, a sample of the liquid medium was diluted in 10% acetonitrile. The concentration of FB1 in the diluted samples was analysed by HPLC-MS/MS, and the results were used to calculate the “pI50 FB1” values.

HPLC-MS/MS was carried out using the parameters below:

Mass spectrometry instrument: Applied Biosystems API4000 QTrap

HPLC: Agilent 1100

Autosampler: CTC HTS PAL

Chromatography column: Waters Atlantis T3 (50×2 mm)

Examples of the Measured pI50 Values

Production of Fumonisin FB1 by Fusarium Proliferatum

| Example | | |
|---|---|---|
| No. of | | pI50 |
| Table I | pI50 Fum | growth |
| 64 | 6.2 | 5.3 |
| 1 | 5.9 | 5.2 |
| 2 | 5.8 | 5.2 |
| 60 | 6.2 | 5.8 |
| 59 | 6.3 | 5.5 |
| 24 | 5.7 | 5.1 |
| 53 | 6.5 | 5.8 |
| 56 | 6.2 | 5.6 |
| 27 | 5.7 | 4.7 |
| 12 | 5.0 | <4.3 |
| 20 | 6.1 | 5.4 |
| 28 | 5.4 | 4.7 |
| 45 | 5.0 | 4.7 |
| 62 | 4.8 | <4.3 |

### Example L

Production of DON/Acetyl-DON by Fusarium Graminearum

The compounds were tested in microtitre plates at 7 concentrations of from 0.07 μM to 50 μM in a DON-inducing liquid medium (1 g of (NH₄)₂HPO₄, 0.2 g of MgSO₄×7 H₂O, 3 g of KH₂PO₄, 10 g of glycerol, 5 g of NaCl and 40 g of sucrose per litre) with oat extract (10%) and DMSO (0.5%). Inoculation was carried out using a concentrated spore suspension of Fusarium graminearum at a final concentration of 2000 spores/ml.

The plate was incubated at high atmospheric humidity at 28° C. for 7 days.

At the beginning and after 3 days, an OD measurement was carried out at OD520 (repeated measurements: 3×3 measurements per well) to calculate the growth inhibition.

After 7 days, 100 μl of an 84/16 acetonitrile/water mixture were added, and samples of the liquid medium were then taken from each well and diluted 1:100 in 10% strength acetonitrile. The proportions of DON and acetyl-DON in the samples were analysed by HPLC-MS/MS, and the measured values were used to calculate the inhibition of the DON/AcDON production compared to an active compound-free control.

The HPLC-MS/MS measurements were carried out using the parameters below:

Ionization type: ESI negative

Ion spray voltage: −4500 V

Spray gas temperature: 500° C.

Decluster potential: −40 V

Collision energy: −22 eV

Collision gas: N₂

NMR trace: 355.0>264.9;

HPLC column: Waters Atlantis T3 (trifunctional C18 bonding, capped)

Particle size: 3 μm

Column dimensions: 50×2 mm

Temperature: 40° C.

Solvent A: water/2.5 mM NH₄OAc+0.05% CH₃COOH (v/v)

Solvent B: methanol/2.5 mM NH₄OAc+0.05% CH₃COOH (v/v)

Flow rate: 400 μl/minute

Injection volume: 11 μl

Gradient:

| Time [min] | A % | B % |
|---|---|---|
| 0 | 100 | 0 |
| 0.75 | 100 | 0 |
| 1.5 | 5 | 95 |
| 4 | 5 | 95 |
| 5 | 100 | 0 |
| 10 | 100 | 0 |

Examples of DON Inhibition

At 50 μM, Examples Nos. 53, 58, 61, 138, 153, 154, 166 and 195 showed a DON/AcDON-inhibitory activity of >80%. The growth inhibition of Fusarium graminearum by the examples with an activity of >80% varied from 87 to 100% at 50 μM.

1. A compound of the formula (I), 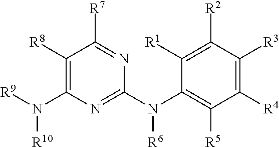
wherein
R¹ to R⁵ independently of one another are hydrogen, C₁-C₄-alkyl, C₁-C₄-alkoxy, C₁-C₄-haloalkyl, C₁-C₄-alkoxy(C₁-C₄)alkyl, C₁-C₄-alkoxy(C₁-C₄)alkoxy or halogen,
where precisely one of the radicals R² or R³ represents a group of the formula E1, E2 or E3, 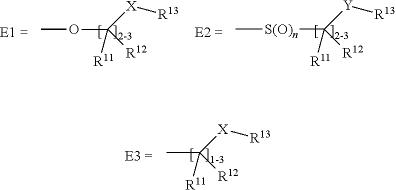
wherein
X is oxygen, NR¹⁴, sulphur, SO or SO₂,
Y is a direct bond, oxygen, NR¹⁴, sulphur, SO or SO₂,
n is 0, 1 or 2,
R⁶ is hydrogen, C₁-C₂-alkyl, C₁-C₄-alkoxy(C₁-C₄)alkyl, C₁-C₄-trialkylsilyl, C₁-C₄-dialkylmonophenylsilyl, formyl, (C₁-C₄-alkyl)carbonyl, (C₁-C₄-alkoxy-C₁-C₄-alkyl)carbonyl, (C₃-C₆-alkenyloxy)carbonyl, (C₃-C₆-cycloalkyl)carbonyl, (halo-C₁-C₄-alkoxy-C₁-C₄-alkyl)carbonyl, (C₁-C₄-haloalkyl)carbonyl, (C₁-C₄-alkoxy)carbonyl, (C₁-C₄-haloalkoxy)carbonyl, benzyloxycarbonyl, unsubstituted or substituted benzyl, unsubstituted or substituted C₂-C₆-alkenyl, unsubstituted or substituted C₂-C₆-alkynyl, C₁-C₂-alkylsulphinyl or C₁-C₂-alkylsulphonyl,
where the substituents independently of one another are selected from among hydrogen, fluorine, chlorine or bromine, C₁-C₄-alkyl, C₁-C₄-alkoxy, hydroxyl, C₁-C₄-haloalkyl, or cyano,
R⁷ is hydrogen, C₁-C₃-alkyl, cyano or C₁-C₃-haloalkyl,
R⁸ is methyl, fluorine, chlorine, bromine, SMe, SOMe, SO₂Me, iodine, CCl₃, CH₂F, CHF₂ or CF₃,
R⁹ is hydrogen, unbranched or branched C₁-C₃-alkyl, 2-methoxyethan-1-yl, prop-2-en-1-yl, C₁-C₄-alkoxy(C₁-C₄)alkyl, unbranched or branched (C₁-C₄-alkyl)carbonyl, (C₁-C₄-haloalkyl)carbonyl, unsubstituted or substituted benzyl, C₁-C₆-trialkylsilyl, C₁-C₄-trialkylsilylethyl, C₁-C₄dialkylmonophenylsilyl, (C₁-C₄-alkoxy)carbonyl, C₁-C₆-alkylsulphinyl, C₁-C₆-alkylsulphonyl, C₁-C₆-haloalkylsulphinyl or C₁-C₆-haloalkylsulphonyl,
where the substituents independently of one another are selected from among hydrogen, halogen, nitro, C₁-C₄-alkyl, C₁-C₄-alkoxy, hydroxyl, C₁-C₄-haloalkyl or cyano,
R¹⁰ is unbranched or branched, unsubstituted or substituted C₁-C₇alkyl, unbranched or branched, unsubstituted or substituted C₂-C₇-haloalkyl, unsubstituted or substituted C₃-C₇-cycloalkyl, unbranched or branched, unsubstituted or substituted C₃-C₇-cycloalkyl(C₁-C₃)alkyl, unbranched or branched, unsubstituted or substituted C₃-C₇-alkenyl, unbranched or branched, unsubstituted or substituted C₃-C₇-alkynyl, unbranched or branched, unsubstituted or substituted C₁-C₄-alkoxy(C₁-C₄)alkyl, unbranched or branched, unsubstituted or substituted C₁-C₄-haloalkoxy(C₁-C₄)alkyl, 2-methyl-1-(methylsulphanyl)propan-2-yl or oxetan-3-yl,
or
R⁹ and R¹⁰ together with the nitrogen atom to which they are bonded form an unsubstituted or substituted 3-7-membered saturated cycle which may contain up to one further heteroatom selected from among oxygen, sulphur or nitrogen,
where the substituents in R¹⁰ independently of one another are selected from among methyl, ethyl, isopropyl, cyclopropyl, fluorine atoms, chlorine atoms and/or bromine atoms, methoxy, ethoxy, methylmercapto, ethylmercapto, cyano, hydroxyl, CF₃,
R¹¹ and R¹² independently of one another are hydrogen, halogen, C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
where the substituents independently of one another are selected from among halogen, C₁-C₄-alkyl or C₁-C₄-haloalkyl,
or
R¹¹ and R¹² together form a methylene group ═CH₂,
R¹³ is hydrogen, C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
where the substituents independently of one another are selected from among halogen, C₁-C₄-alkyl or C₁-C₄-haloalkyl,
R¹⁴ is hydrogen, C₁-C₆-alkyl, C₁-C₄-alkoxy(C₁-C₄)alkyl or unsubstituted or substituted benzyl,
where the substituents independently of one another are selected from among halogen, C₁-C₄-alkyl or C₁-C₄-haloalkyl,
and agrochemically active salts thereof.

2. The compound of formula (I), according to claim 1,
wherein
R¹ to R⁵ independently of one another are hydrogen, C₁-C₃-alkyl, C₁-C₃-alkoxy, C₁-C₂-haloalkyl, C₁-C₄-alkoxy(C₁-C₄)alkyl, C₁-C₄-alkoxy(C₁-C₄)alkoxy or halogen,
where precisely one of the radicals R² or R³ represents a group of the formula E1, E2 or E3, 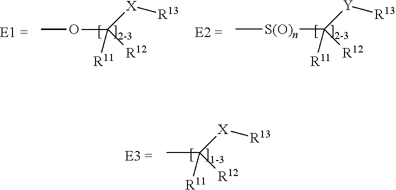
wherein
X is oxygen, NR¹⁴, sulphur, SO or SO₂,
Y is a direct bond, oxygen, NR¹⁴, sulphur, SO or SO₂,
n is 0, 1 or 2,
R⁶ is hydrogen, C₁-C₂-alkyl, C₁-C₄-alkoxy(C₁-C₄)alkyl, C₁-C₄-trialkylsilyl, formyl, (C₁-C₄-alkyl)carbonyl, (C₁-C₄-alkoxy-C₁-C₄-alkyl)carbonyl, (C₃-C6-cycloalkyl)carbonyl , (C₁-C₄-haloalkyl)carbonyl, (C₁-C₄-alkoxy)carbonyl, benzyloxycarbonyl, unsubstituted or substituted benzyl, unsubstituted or substituted C₂-C₆-alkenyl, unsubstituted or substituted C₂-C₆-alkynyl or C₁-C₂-alkylsulphonyl,
where the substituents independently of one another are selected from among fluorine, chlorine or bromine, C₁-C₂-alkyl, C₁-C₂-alkoxy, hydroxyl, C₁-C₂-haloalkyl, or cyano,
R⁷ is hydrogen, C₁-C₃-alkyl, cyano or C₁-C₃-haloalkyl,
R⁸ is methyl, fluorine, chlorine, bromine, SMe, SOMe, SO₂Me, iodine, CCl₃, CH₂F, CHF₂ or CF₃,
R⁹ is hydrogen, methyl, ethyl, propyl, propan-2-yl, butyl, pentyl, hexyl, 2-methoxyethan-1-yl, 2,2,2-trifluoroethyl, prop-2-en-1-yl, CH₂OCH₃, COMe, COOMe, COOEt, COOtertBu, COCF3, benzyl or SO₂CH₃,
R¹⁰ is unbranched or branched, unsubstituted or substituted C₁-C₆-alkyl, unbranched or branched, unsubstituted or substituted C₃-C₆-cycloalkyl(C₁-C₂)alkyl, unsubstituted or substituted C₃-C₆-cycloalkyl, unbranched or branched, unsubstituted or substituted C₃-C₄-alkenyl, unbranched or branched, unsubstituted or substituted C₃-C₄-alkynyl, unbranched or branched, unsubstituted or substituted C₂-C₄-haloalkyl, unbranched or branched, unsubstituted or substituted C₁-C₂-alkoxy(C₁-C₄)alkyl, unbranched or branched, unsubstituted or substituted C₁-C₂-alkylmercapto(C₁-C₄)alkyl, or oxetan-3-yl,
where the substituents in R¹⁰ independently of one another are selected from among methyl, ethyl, isopropyl, cyclopropyl, fluorine atoms, chlorine atoms and/or bromine atoms, methoxy, ethoxy, methylmercapto, ethylmercapto, cyano, hydroxyl or CF₃,
or
R⁹ and R¹⁰ together with the nitrogen atom to which they are bonded form an azetidinyl, pyrrolidinyl, piperidinyl, morpholinyl, azepanyl, 4-methylpiperazin-1-yl, 2-methylpiperidin-1-yl, -methylpyrrolidin-1-yl, 2-methylazetidin-1-yl or thiomorpholinyl ring,
R¹¹ and R¹² independently of one another are hydrogen, halogen, C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy (C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
where the substituents independently of one another are selected from among halogen, C₁-C₄-alkyl or C₁-C₄-haloalkyl,
or
R¹¹ and R¹² together form a methylene group ═CH₂,
R¹³ is hydrogen, C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
where the substituents independently of one another are selected from among halogen, C₁-C₄-alkyl or C₁-C₄-haloalkyl,
R¹⁴ is hydrogen, C₁-C₆-alkyl, C₁-C₄-alkoxy(C₁-C₄)alkyl or unsubstituted or substituted benzyl,
where the substituents independently of one another are selected from among halogen, C₁-C₄-alkyl or C₁-C₄-haloalkyl,
and the agrochemically active salts thereof.

3. The compound of the formula (I) according to claim 1,
wherein
R¹ to R⁵ independently of one another are hydrogen, methyl, ethyl, fluorine, chlorine, bromine, iodine, trifluoromethyl, difluoromethyl, OCH₃, OCH₂CH₃, O(CH₂)₂OCH₃, CH₂OCH₃ or CH₂OCH₂CH₃,
where precisely one of the radicals R² or R³ represents a group of the formula E1, E2 or E3, 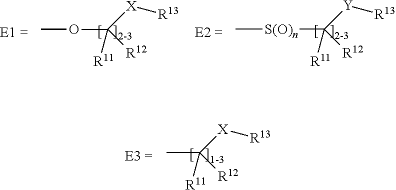
wherein
X is oxygen, NR¹⁴, sulphur, SO or SO₂,
Y is a direct bond, oxygen, NR¹⁴, sulphur, SO or SO₂,
n is 0, 1 or 2,
R⁶ is hydrogen, Me, benzyl, SO₂CH₃, COMe, COCF₃, COOMe or CHO,
R⁷ is hydrogen, methyl, cyano, CHF₂ or CF₃,
R⁸ is methyl, fluorine, chlorine, bromine, SMe, SOMe, SO₂Me, iodine, CCl₃, CHF₂ or CF₃,
R⁹ is hydrogen, methyl, ethyl, propyl, propan-2-yl, butyl, pentyl, hexyl, 2-methoxyethan-1-yl, 2,2,2-trifluoroethyl, prop-2-en-1-yl, CH₂OCH₃, COMe, COOMe, COOEt, COOtertBu, COCF₃, benzyl or SO₂CH₃,
R¹⁰ is methyl, ethyl, propyl, isopropyl, butyl, tert-butyl, 2-methylprop-1-yl, butan-2-yl, pentyl, 2,2-dimethylprop-1-yl, 2-methylbut-l-yl, 3-methylbut-1-yl, 3-methylbutan-2-yl, pentan-2-yl, pentan-3-yl, hexyl, 2,2-dimethyl-butan-2-yl, prop-2-en-1-yl, 2-methylprop-2-en-1-yl, prop-2-yn-1-yl, 2-fluoroethan-1-yl, 1-fluoropropan-2-yl, 3-fluoropropan-1-yl, 2,2-difluoroethyl, 2,2,2-trifluoroethyl, 2,2-difluoropropan-1-yl, 1,1,1-trifluoropropan-2-yl, 3,3,3-trifluoropropan-1-yl, 2,2,3,3,3-pentafluoropropyl, 1,1,1-trifluorobutan-2-yl, 1,1,1-trifluoro-butan-3-yl, 1,1,1-trifluoro-2-methylpropan-2-yl, 1-fluoro-2-methyl-propan-2-yl, 1,1,1-trifluoro-3-methylbutan-2-yl, 2-chloroethan-1-yl, cyanomethyl, 2-methoxyethan-1-yl, 3-methoxypropan-1-yl, 2-methyl-mercaptoethan-1-yl, 1-methylmercaptopropan-2-yl, cyclopropyl, cyclo-butyl, cyclopentyl, cyclohexyl, oxetan-3-yl, cyclopropylmethyl, 1-cyclopropyleth-1-yl, 2-methylcyclopropyl, 2,2-dimethylcyclopropyl, 2-methylcyclobut-1-yl, 3-methylcyclobut-1-yl, 2-methyl-3-oxobutan-2-yl or 3-(2-oxoazepan-1-yl)propyl,
or
R⁹ and R¹⁰ together with the nitrogen atom to which they are bonded form an aziridinyl, azetidinyl, pyrrolidinyl, piperidinyl, azepanyl, piperazin-1-yl, 4-methylpiperazin-1-yl, morpholinyl or thiomorpholinyl ring,
R¹¹ and R¹² independently of one another are hydrogen, methyl, ethyl, propyl, isopropyl, butyl, pentyl, hexyl, cyclopropyl, cyclobutyl, cyclopentyl, cyclohexyl, chlorine, trifluoromethyl, (CH₂)₂OCH₃, phenyl or benzyl,
or
R¹¹ and R¹² together form a methylene group ═CH₂,
R¹³ is hydrogen, methyl, ethyl, propyl, isopropyl, butyl, pentyl, hexyl, 2,2,2-trifluoroethyl, cyclopropyl, cyclobutyl, cyclopentyl, cyclohexyl, (CH₂)₂OCH₃, phenyl or benzyl,
R¹⁴ is hydrogen, methyl, ethyl, propyl, isopropyl, butyl, pentyl, (CH₂)₂OCH₃ or benzyl,
and the agrochemically active salts thereof.

4. The compound of the formula (I) according to claim 1, wherein
R¹ to R⁵ independently of one another are hydrogen, methyl, ethyl, fluorine, chlorine, bromine, iodine, trifluoromethyl, difluoromethyl, OCH₃, OCH₂CH₃, O(CH₂)₂OCH₃, CH₂OCH₃ or CH₂OCH₂CH₃,
where precisely one of the radicals R² or R³ represents a group of the formula E1, E2 or E3, 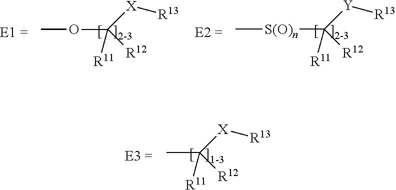
wherein
X is oxygen, sulphur, SO or SO₂,
Y is a direct bond, oxygen, sulphur, SO or SO₂,
n is 0, 1 or 2,
R⁶ is hydrogen, Me, COMe or CHO,
R⁷ is hydrogen or methyl,
R⁸ is methyl, fluorine, chlorine, bromine, SMe, SOMe, SO₂Me, CHF₂ or CF₃,
R⁹ is hydrogen, methyl, ethyl, propyl, propan-2-yl, butyl, pentyl, 2-methoxyethan-1-yl, 2,2,2-trifluoroethyl, prop-2-en-1-yl, CH₂OCH₃, COMe, COOMe, COOEt, COOtertBu, COCF₃ or benzyl,
R¹⁰ is methyl, ethyl, propyl, isopropyl, butyl, tert-butyl, 2-methylprop-1-yl, butan-2-yl, pentyl, 2,2-dimethylprop-1-yl, 2-methylbut-1-yl, 3-methylbut-1-yl, pentan-2-yl, pentan-3-yl, hexyl, prop-2-en-1-yl, 2-methylprop-2-en-1-yl, prop-2-yn-1-yl, 2-fluoroethan-1-yl, 1-fluoropropan-2-yl, 2,2-difluoroethyl, 2,2,2-trifluoroethyl, 1,1,1-trifluoropropan-2-yl, 3,3,3-trifluoropropan-1-yl, 2,2,3,3,3-pentafluoropropyl, 1,1,1-trifluoro-butan-2-yl, 1,1,1-trifluorobutan-3-yl, 2-chloroethan-1-yl, cyanomethyl, 2-methoxyethan-1-yl, 3-methoxypropan-1-yl, 2-methylmercaptoethan-1-yl, 1-methylmercaptopropan-2-yl, cyclopropyl, cyclobutyl, cyclopentyl, cyclohexyl, oxetan-3-yl, cyclopropylmethyl, 1-cyclopropyleth-1-yl, 2-methylcyclopropyl, 2,2-dimethylcyclopropyl, 2-methylcyclobut-1-yl, 3-methylcyclobut-1-yl, 2-methyl-3-oxobutan-2-yl or 3-(2-oxo-azepan-1-yl)propyl,
or
R⁹ and R¹⁰ together with the nitrogen atom to which they are bonded form an aziridinyl, azetidinyl, pyrrolidinyl, piperidinyl, azepanyl, piperazin-1-yl, 4-methylpiperazin-1-yl, morpholinyl or thiomorpholinyl ring,
R¹¹ and R¹² independently of one another are hydrogen, methyl, ethyl, propyl, isopropyl, butyl, pentyl, hexyl, chlorine, cyclopropyl, trifluoromethyl, phenyl or benzyl,
or
R¹¹ and R¹² together form a methylene group ═CH₂,
R¹³ is hydrogen, methyl, ethyl, propyl, isopropyl, butyl, pentyl, hexyl, 2,2,2-trifluoroethyl, (CH₂)₂OCH₃, phenyl or benzyl
and the agrochemically active salts thereof.

5. The compound of the formula (I) according to claim 1, wherein
R¹ and R⁵ independently of one another are hydrogen or F,
R² is hydrogen, fluorine, chlorine, trifluoromethyl, O(CH₂)₂OCH₃, O(CH₂)₂OCH₂CH₃, O(CH₂)₂OCH₂CH₂CH₃, O(CH₂)₃OCH₃, O(CH₂)₃OCH₂CH₃, O(CH₂)₂O(CH₂)₂OCH₃, (1-methoxypropan-2-yl)oxy, CH₂OCH₃, CH₂OCH₂CH₃, CH₂OCH₂CH₂CH₃, 1-methoxyethyl, 1-ethoxyethyl, 1-propoxyethyl, 2,2,2-trifluoro-1-methoxyethyl, 2,2,2-trifluoro-1-ethoxyethyl, 2-methoxypropan-2-yl, 2-ethoxy-propan-2-yl, phenoxymethyl, (CH₂)₂OCH₃, (CH₂)₂OCH₂CH₃, 2-methoxy-2-methylpropyl, 2-ethoxy-2-methylpropyl, SEt, SOEt, SO₂Et, SPr, SOPr, SO₂Pr, SisoPr, SOisoPr, SO₂isoPr, SBu, SOBu, SO₂Bu, SisoBu, SOisoBu, SO₂isoBu, SsecBu, SOsecBu, SO₂secBu, (2-methylprop-2-en-1-yl)-sulphanyl, (2-chloroethyl)sulphonyl, (methylsulphanyl)methyl, (methyl-sulphinyl)methyl, (methylsulphonyl)methyl, (ethylsulphanyl)methyl, (ethylsulphinyl)methyl, (ethylsulphonyl)methyl, (propylsulphanyl)methyl, (propylsulphinyl)methyl, (propylsulphonyl)methyl, 1-(methylsulphanyl)-ethyl, 1-(methylsulphinyl)ethyl, 1-(methylsulphonyl)ethyl, 1-(ethyl-sulphanyl)ethyl, 1-(ethylsulphinyl)ethyl, 1-(ethylsulphonyl)ethyl, 1-(propylsulphanyl)ethyl, 1-(propylsulphinyl)ethyl, 1-(propylsulphonyl)-ethyl, 1-(isopropylsulphanyl)ethyl, 1-(isopropylsulphinyl)ethyl, 1-(isopropylsulphonyl)ethyl, 1-(sec-butylsulphanyl)ethyl, 1-(sec-butyl-sulphinyl)ethyl, 1-(sec-butylsulphonyl)ethyl, 1-(pentan-2-ylsulphanyl)-ethyl, 1-(pentan-2-ylsulphinyl)ethyl, 1-(pentan-2-ylsulphonyl)ethyl, 1-(methylsulphanyl)propyl, 1-(methylsulphinyl)propyl, 1-(methyl-sulphonyl)propyl, 1-(ethylsulphanyl)propyl, 1-(ethylsulphinyl)propyl, 1-(ethylsulphonyl)propyl, 2-(methylsulphanyl)ethyl, 2-(methylsulphinyl)-ethyl, 2-(methylsulphonyl)ethyl, 2-(ethylsulphanyl)ethyl, 2-(ethyl-sulphinyl)ethyl or 2-(ethylsulphonyl)ethyl,
R³ is hydrogen, methyl, fluorine, chlorine, bromine, trifluoromethyl, O(CH₂)₂OCH₃, O(CH₂)₂OCH₂CH₃, O(CH₂)₃OCH ₃, O(CH₂)₃OCH₂CH₃, O(CH₂)₂O(CH₂)₂OCH₃, (1-methoxypropan-2-yl)oxy, CH₂OCH₃, CH₂OCH₂CH₃, CH₂OCH₂CH₂CH₃, 1-methoxyethyl, 1-ethoxyethyl, 1-propoxyethyl, 2-methoxypropan-2-yl, 2-ethoxypropan-2-yl, (CH₂)₂OCH₃, (CH₂)₂OCH₂CH₃, 2-methoxy-2-methylpropyl, 2-ethoxy-2-methylpropyl, SEt, SOEt, SO₂Et, SPr, SOPr, SO₂Pr, SisoPr, SOisoPr, SO₂isoPr, SBu, SOBu, SO₂Bu, Siso-u, SOisoBu, SO₂isoBu, SsecBu, SOsecBu, SO₂secBu, (methylsulphanyl)methyl, (methylsulphinyl)methyl, (methylsulphonyl)methyl, (ethylsulphanyl)methyl, (ethylsulphinyl)-methyl, (ethylsulphonyl)methyl, 1-(methylsulphanyl)ethyl, 1-(methyl-sulphinyl)ethyl, 1-(methylsulphonyl)ethyl, 1-(ethylsulphanyl)ethyl, 1-(ethylsulphinypethyl, 1-(ethylsulphonyl)ethyl, 1-(propylsulphanyl)-ethyl, 1-(propylsulphinyl)ethyl, 1-(propylsulphonypethyl, 1-(methyl-sulphanyl)propyl, 1-(methylsulphinyl)propyl, 1-(methylsulphonyl)propyl, 1-(ethylsulphanyl)propyl, 1-(ethylsulphinyl)propyl, 1-(ethylsulphonyl)-propyl, 2-(methylsulphanyl)ethyl, 2-(methylsulphinyl)ethyl or 2-(methyl-sulphonyl)ethyl,
where R² and R³ are not simultaneously hydrogen,
with the proviso that, if R² is other than hydrogen, fluorine, chlorine or trifluoromethyl,
R³ may only have one of the following meanings:
hydrogen, methyl, fluorine, chlorine, bromine or trifluoromethyl,
R⁴ is hydrogen, methyl, fluorine, chlorine, trifluoromethyl, O(CH₂)₂OCH₃, CH₂OCH₃ or CH₂OCH₂CH₃,
R⁶ is hydrogen or CHO,
R⁷ is hydrogen,
R⁸ is fluorine, chlorine, bromine, SMe, SOMe, SO₂Me or CF₃,
R⁹ is hydrogen, methyl, ethyl, propyl, propan-2-yl, 2-methoxyethan-1-yl or prop-2-en-1-yl,
R¹⁰ is methyl, ethyl, propyl, isopropyl, butyl, tert-butyl, 2-methylprop-1-yl, pentyl, 2,2-dimethylprop-1-yl, 2-methylbut-1-yl, 3-methylbut-1-yl, pentan-2-yl, pentan-3-yl, prop-2-en-1-yl, prop-2-yn-1-yl, 2-fluoroethan-1-yl, 2,2-difluoroethyl, 2,2,2-trifluoroethyl, 2-chloroethan-1-yl, cyanomethyl, cyclopropyl, cyclobutyl, cyclopentyl, cyclopropylmethyl, 1-cyclopropyleth-1-yl, 2-methylcyclopropyl, 2-methyl-3-oxobutan-2-yl or 3-(2-oxoazepan-1-yl)propyl,
or
R⁹ and R¹⁰ together with the nitrogen atom to which they are bonded form a pyrrolidinyl, piperidinyl, azepanyl, 4-methylpiperazin-1-yl, morpholinyl or thiomorpholinyl ring,
and the agrochemically active salts thereof.

6. A composition for controlling phytopathogenic harmful fungi, comprising at least one compound of formula (I) according to claim 1, and one or more extenders, surfactants or a combination thereof.

7. (canceled)

8. A method of controlling phytopathogenic harmful fungi, comprising applying the compound of formula (I) according to claim 1 to one or more phytopathogenic harmful fungi, their habitat or a combination thereof.

9. A process for preparing a composition for controlling phytopathogenic harmful fungi, comprising mixing a compound of formula (I) according claim 1 with one or more extenders, surfactants or a combination thereof.

10. A process for preparing the compound of the formula (I), (Ia), (Ib) and (Ic) according to the invention, comprising at least one of steps (a) to (k) below:
(a) reacting a compound of formula (III) with a compound of formula (II) in the presence of a base, if appropriate in the presence of a solvent and if appropriate in the presence of a catalyst, to give a compound of the formula (V), according to the reaction scheme below: 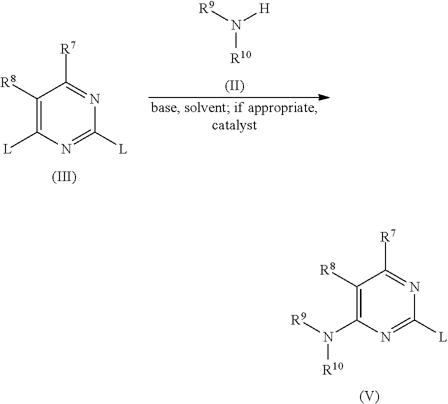
where L=F, Cl, Br or I;
(b) reacting the compound of the formula (V) with a compound of formula (IVa), (IVb) or (IVc), if appropriate in the presence of an acid or if appropriate in the presence of a base, if appropriate in the presence of a solvent, according to the reaction schemes below: 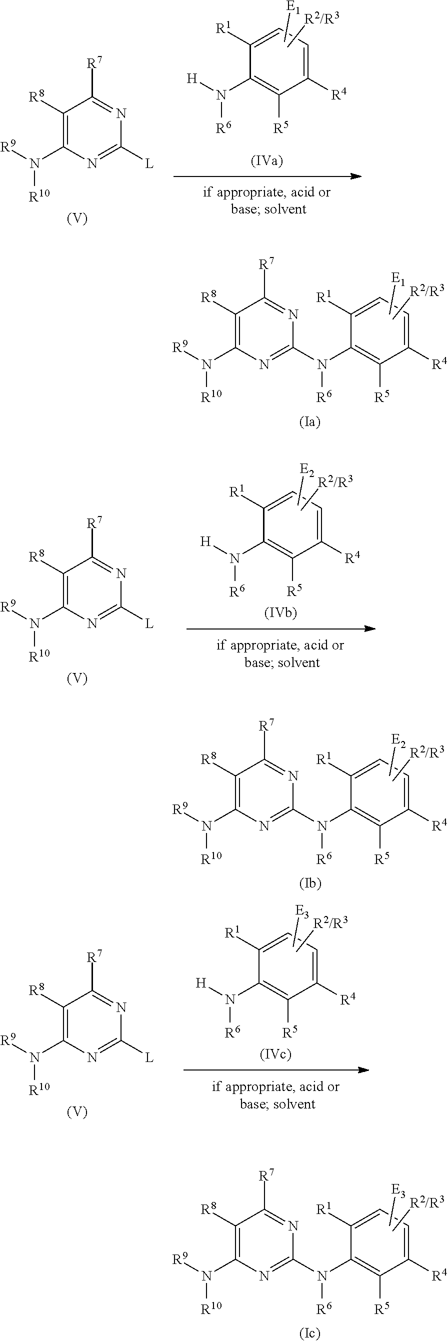
where L=F, Cl, Br or I;
(c) oxidizing a compound of formula (Ib-I) with a suitable oxidant to give a compound of the formula (Ib-II) in the presence of a solvent according to the reaction scheme below: 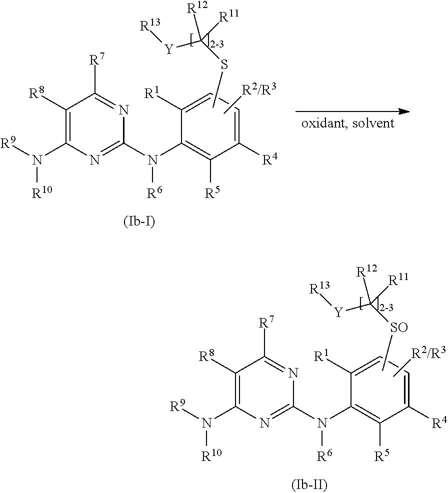
(d) oxidizing the compound of formula (Ib-I) with a suitable oxidant to give a compound of formula (Ib-III) in the presence of a solvent according to the reaction scheme below: 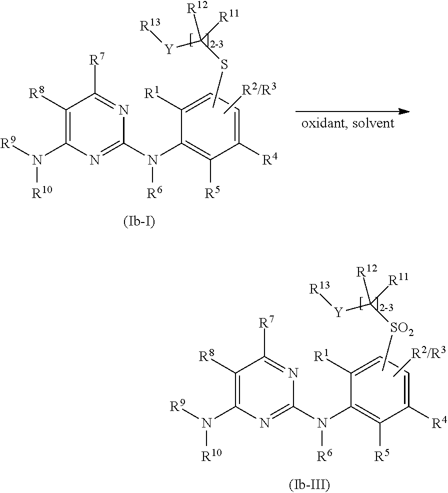
(e) oxidizing a compound of formula (Ic-I) with a suitable oxidant to give a compound of formula (Ic-II) in the presence of a solvent according to the reaction scheme below: 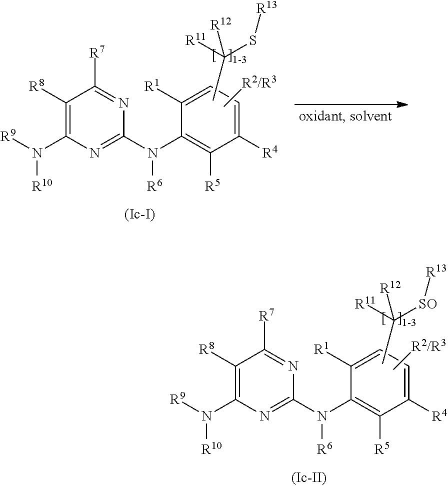
oxidizing the compound of formula (Ic-I) with a suitable oxidant to give a compound of formula (Ic-III) in the presence of a solvent according to the reaction scheme below: 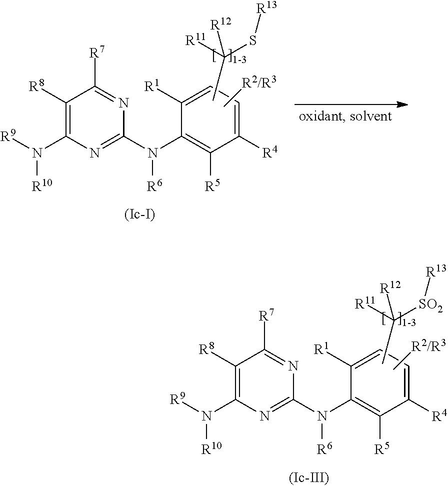
(g) reducing a compound of formula (VIa), (VIb) or (VIc) to give a compound of the formula (IVa), (IVb) or (IVc) by means of a suitable reducing agent, if appropriate in the presence of an acid and in the presence of a solvent, according to the reaction schemes below: 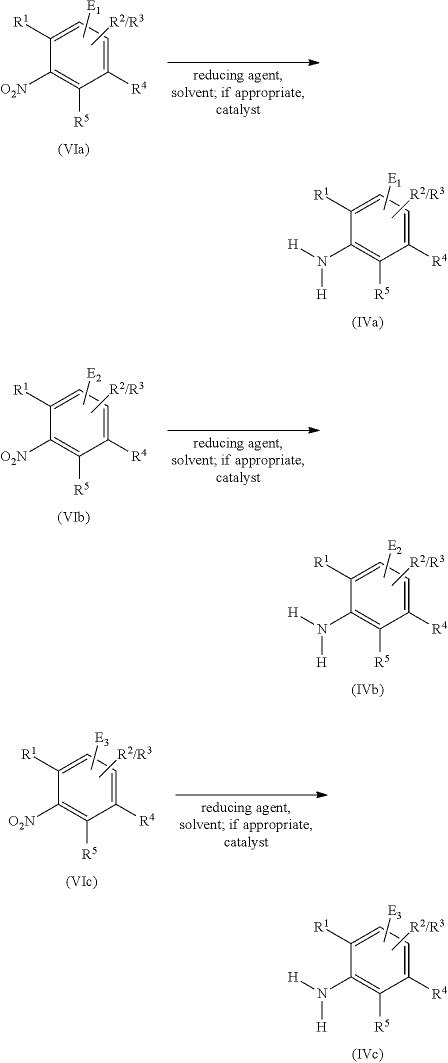
(h) reacting a compound of the formula (VII) with a suitable thiol to give a compound of the formula (VIc-I), if appropriate in the presence of a solvent, according to the reaction scheme below: 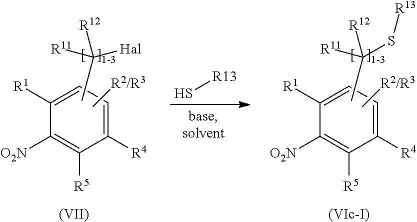
(i) reacting a compound of formula (VIc-II) with a suitable chlorinating agent to give a compound of formula (VIIa), if appropriate in the presence of a solvent, if appropriate in the presence of a suitable catalyst and if appropriate in the presence of a suitable base, according to the reaction scheme below: 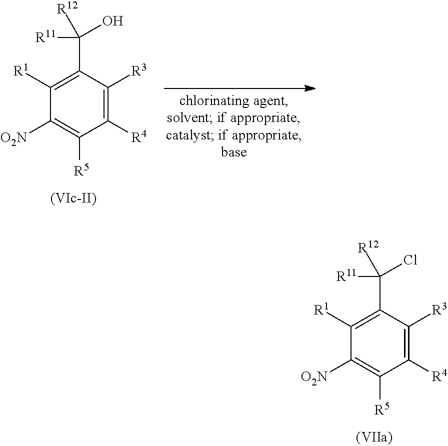
(j) reacting a compound of the formula (X) with the compound of formula (II) to give the compound of formula (I) in the presence of a base, if appropriate in the presence of a solvent and if appropriate in the presence of a catalyst, according to the reaction scheme below: 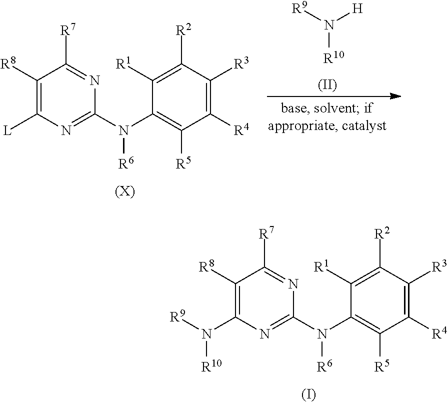
(k) reacting the compound of formula (III) with a compound of the formula (IV) to give the compound of formula (X) in the presence of a base, or in the presence of a Lewis acid, if appropriate in the presence of a solvent and if appropriate in the presence of a catalyst, according to the reaction scheme below: 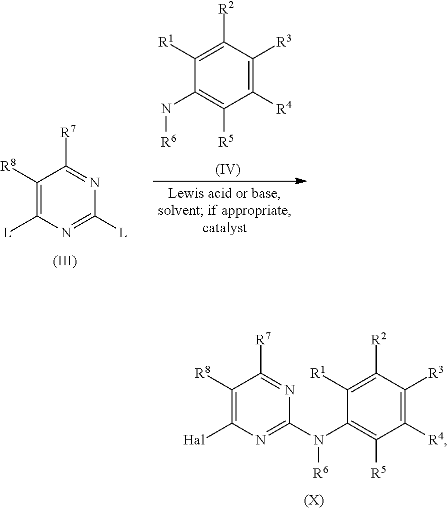
where the definitions of the radicals R¹ to R¹³ and X and Y in the above schemes correspond to the abovementioned definitions of claim 1, and L represents F, Cl, Br or I.

11. A compound of formula (Va), (Vb) and (Vc), 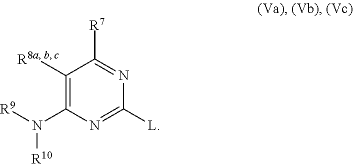
wherein
R^{8a} represents iodine, CFH₂, CF₂H, CCl₃, cyano or Me,
R^{8b} represents CF₃,
R^{8c} represents Br,
L is F, Cl, Br or I
and
R⁷, R⁹, and R¹⁰, have the meanings of claim 1.

12. The compound of the formula (IVc-I), 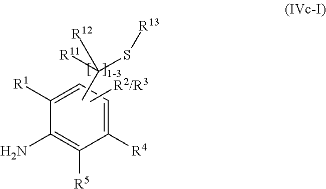
wherein
R¹ and R⁵ represent hydrogen,
R¹¹ represents C₁-C₆-alkyl, unsubstituted or substituted C₃-C₆-cycloalkyl, unsubstituted or substituted C₃-C₆-cycloalkyl(C₁-C₄)alkyl, C₁-C₃-haloalkyl, C₁-C₄-alkoxy(C₁-C₄)alkyl, unsubstituted or substituted C₂-C₄-alkenyl, unsubstituted or substituted C₂-C₄-alkynyl, unsubstituted or substituted phenyl or unsubstituted or substituted benzyl,
where the substituents independently of one another are selected from among halogen, C₁-C₄-alkyl or C₁-C₄-haloalkyl,
and
R² to R⁴, R¹², and R¹³ have the meanings of claim 1.

13. The compound of the formula (X), 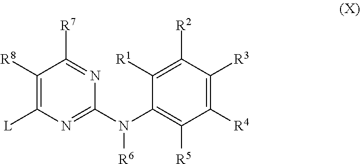
wherein
R¹ to R⁸ have the meanings of claim 1,
L represents fluorine, chlorine, bromine or iodine.

14. The compound of the formula (VIIa), 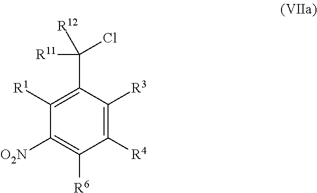
wherein
R¹ , R⁵ and R¹¹ represent hydrogen,
R¹² represents unbranched C₂-C₆-alkyl or C₃-C₆-cycloalkyl,
and
R³ R⁴ have the meanings of claim 1.

15. A composition for controlling phytopathogenic harmful fungi, comprising at least one compound of formula (I) according to claim 2, and one or more extenders, surfactants or a combination thereof.

16. A composition for controlling phytopathogenic harmful fungi, comprising at least one compound of formula (I) according to claim 3, and one or more extenders, surfactants or a combination thereof.

17. A composition for controlling phytopathogenic harmful fungi, comprising at least one compound of formula (I) according to claim 4, and one or more extenders, surfactants or a combination thereof.

18. A composition for controlling phytopathogenic harmful fungi, comprising at least one compound of formula (I) according to claim 5, and one or more extenders, surfactants or a combination thereof.

19. A method of controlling phytopathogenic harmful fungi, comprising applying the compound of formula (I) according to claim 2 to one or more phytopathogenic harmful fungi, their habitat or a combination thereof.

20. A method of controlling phytopathogenic harmful fungi, comprising applying the compound of formula (I) according to claim 3 to one or more phytopathogenic harmful fungi, their habitat or a combination thereof.

21. A method of controlling phytopathogenic harmful fungi, comprising applying the compound of formula (I) according to claim 4 to one or more phytopathogenic harmful fungi, their habitat or a combination thereof.

22. A method of controlling phytopathogenic harmful fungi, comprising applying the compound of formula (I) according to claim 5 to one or more phytopathogenic harmful fungi, their habitat or a combination thereof.
